SEQUENCE LISTING (38 sequences)

SEQ ID NO: 1 - protein, 178 aa, Homo Sapiens [sequence omitted]

SEQ ID NO: 2 - protein, 17 aa, Artificial sequence
  Synthesized CENP001
LKDTTSSSSADSTIMDC

SEQ ID NO: 3 - protein, 15 aa, Artificial sequence [sequence omitted]

SEQ ID NO: 4 - protein, 15 aa, Artificial sequence
  Synthesized CENP005
MDIQVPTRAPDAVYC

SEQ ID NO: 5 - protein, 15 aa, Artificial sequence
  Synthesized CENP006
APDAVYTELQPTSPC

SEQ ID NO: 6 - protein, 15 aa, Artificial sequence
  Synthesized CENP007
LQPTSPTPTWPADEC

SEQ ID NO: 7 - protein, 15 aa, Artificial sequence
  Synthesized CENP008
TWPADETPQPQTQTC

SEQ ID NO: 8 - protein, 15 aa, Artificial sequence
  Synthesized CENP009
QPQTQTQQLEGTDGC

SEQ ID NO: 9 - protein, 15 aa, Artificial sequence
  Synthesized CENP010
LEGTDGPLVTDPETC

SEQ ID NO: 10 - protein, 15 aa, Artificial sequence
  Synthesized CENP011
VTDPETHKSTKAAHC

SEQ ID NO: 11 - protein, 15 aa, Artificial sequence
  Synthesized CENP012
STKAAHPTDDTTTLC

SEQ ID NO: 12 - protein, 15 aa, Artificial sequence
  Synthesized CENP013
DDTTTLSERPSPSTC

SEQ ID NO: 13 - protein, 15 aa, Artificial sequence
  Synthesized CENP014
RPSPSTDVQTDPQTC

SEQ ID NO: 14 - protein, 15 aa, Artificial sequence
  Synthesized CENP015
QTDPQTLKPSGFHEC

SEQ ID NO: 15 - protein, 15 aa, Artificial sequence
  Synthesized CENP016
PSGFHEDDPFFYDEC

SEQ ID NO: 16 - protein, 15 aa, Artificial sequence
  Synthesized CENP017
DEPFFYDEHTLRKRC

SEQ ID NO: 17 - protein, 14 aa, Artificial sequence
  Synthesized CENP018
PTRAPDAVYTELQC

SEQ ID NO: 18 - DNA, 22 bp, Artificial sequence
  Synthesized universal primer
ctaatacgactcactatagggc

SEQ ID NO: 19 - DNA, 26 bp, Artificial sequence
  Synthesized IgG1/IgG2A primer
ctcaattttcttgtccaccttggtgc

SEQ ID NO: 20 - DNA, 26 bp, Artificial sequence
  Synthesized IgG2b primer
ctcaagttttttgtccaccgtggtgc

SEQ ID NO: 21 - DNA, 827 bp, Artificial sequence
  Synthesized M53 HC coding sequence including signal peptide, variable domain, and portion of constant domain
tgggccctctagatgcatgctcgagcggccgccagtgtgatggataacggatccgaattgccccttctaatacgactcactatagggcaagcagtggtatcaacgcagagtacatggggaggcagagaactttagccctgtcttcccttttagtgttcagcactgacaatataacattgaacatgctgtcggggctgaagtgggttttctttgttgttttttatcaaggtgtgcattgtgaggtgcagcttgttgagtctggtggaggattggtgcagcctaaagggacattgaaactctcatgtgccgcctctggattcagcttcaatacccatgccatgaactgggtccgccaggctccaggaaagagtttggaatgggttgctcgcacaatgagtaaaagtaataattatgcaacatattatgcagattcagtgaaagatagattcatcatctccagagatgattcacaaagcatgctctatctgcaaatgaacaacttgaaaactgaggacacagccatgtattactgtgtgagggacgaccctaagagaggtatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggccc

SEQ ID NO: 22 - DNA, 787 bp, Artificial sequence
  Synthesized M53 LC coding sequence including signal peptide, variable domain, and portion of constant domain
gggagactcctatagggcgattgggccctctagatgcatgctcgagcggccgccagtgtgatggatatcggatccgaattgccccttctaatacgactcactatagggcaagcagtggtatcaacgcagagtacatggggagacaggcaggggaagcaagatggattcacaggcccaggttcttatgttactgctgctatgggtatctggtacctgtgggggcattgtgatgtcacagtctccatcctccctagctgtgtcagttggagagaaggttactatgagctgcaagcccagtcagagccttttatatagtcgcaatcaaaagatctacttggcctggtaccagcagaaaccagggcagtctcctaaactgctgatttactgggcatccactagggaatctggggtccctgatcgcttcacaggcagtggatctgggacagatttcactctcatcatcagcagtgtgagggctgaagacctggcagtttattactgtcagcaatattataactatcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaag

SEQ ID NO: 23 - protein, 450 aa, Artificial sequence
  Synthesized chimeric M53-HC
ECQLVESGGGLVQPKGTLKLSCAASGFSFNTHAMNWVRQAPGKSLEWVARTMSKSNNYATYYADSVKDRFIISRDDSQSMLYLQMNNLKTEDTAMYYCVRDDPKRGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 220 aa, Artificial sequence
  Synthesized chimeric M53-LC
GIVMSQSPSSLAVSVGEKVTMSCKPSQSLLYSRNQKIYLAWYQQKPGQSPKLLIYWASTRESGVPDRFTGSGSGTDFTLIISSVRAEDLAVYYCQQYYNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 1443 bp, Artificial sequence
  Synthesized chimeric M53-HC coding sequence including signal sequence
atggacagcaaaggttcgtcgcagaaagggtcccgcctgctcctgctgctggtggtgtcaaatctactcttgtgccagggtgtggtctccgagtgccagcttgttgagtctggtggaggattggtgcagcctaaagggacattgaaactctcatgtgccgcctctggattcagcttcaatacccatgccatgaactgggtccgccaggctccaggaaagagtttggaatgggttgctcgcacaatgagtaaaagtaataattatgcaacatattatgcagattcagtgaaagatagattcatcatctccagagatgattcacaaagcatgctctatctgcaaatgaacaacttgaaaactgaggacacagccatgtattactgtgtgagggacgaccctaagagaggtatggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccccgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - DNA, 723 bp, Artificial sequence
  Synthesized chimeric M53-LC coding sequence including signal sequence
atggattcacaggcccaggttcttatgttactgctgctatgggtatctggtacctgtgggggcattgtgatgtcacagtctccatcctccctagctgtgtcagttggagagaaggttactatgagctgcaagcccagtcagagccttttatatagtcgcaatcaaaagatctacttggcctggtaccagcagaaaccagggcagtctcctaaactgctgatttactgggcatccactagggaatctggggtccctgatcgcttcacaggcagtggatctgggacagatttcactctcatcatcagcagtgtgagggctgaagacctggcagtttattactgtcagcaatattataactatcctctcacgttcggtgctgggaccaagctggagctgaaacgtaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 27 - protein, 30 aa, Artificial sequence
  Synthesized chimeric M53-HC signal peptide
MDSKGSSQKGSRLLLLLVVSNLLLCQGVVS

SEQ ID NO: 28 - protein, 20 aa, Artificial sequence
  Synthesized chimeric M53-LC signal peptide
MDSQAQVLMLLLLWVSGTCG

SEQ ID NO: 29 - DNA, 833 bp, Artificial sequence
  Synthesized CENscFv003 [Cysteine on heavy chain] coding sequence
aggaggtaaaacatatgaaacaaagcaccattgcactggcactgctgccgctgctgtttacgccggtcacgaaggcggaatgccagctggttgagtccggcggtggtctggtgcaaccgaaaggtaccctgaagttgagctgtgccgcctcgggcttttcgttcaacacccacgcgatgaactgggtccgccaagcgcctggcaagagcctggagtgggttgcgcgtaccatgagcaagagcaataactatgctacctactatgccgacagcgtcaaagaccgtttcattatcagccgcgatgacagccagagcatgctgtatttgcagatgaataatctgaaaaccgaggacactgcaatgtattactgtgtgcgtgatgatccgaagcgcggcatggactactggggtcagggcaccagcgttacggtgagctctggtggtggcggcagcggtggtggcggtagcggtggcggtggcagcggtattgttatgtctcagagcccgagctccctggcggttagcgtgggtgaaaaagtgacgatgagctgcaaaccgagccaatctctgttgtacagccgcaaccagaagatctatctggcgtggtaccaacaaaagccgggtcagagcccaaaactgctgatctattgggcatccacgcgtgagtccggtgtcccggatcgtttcacgggttctggttccggcaccgacttcactctgatcattagcagcgttcgtgcggaggatctggcagtgtactattgccagcaatactacaattacccgctgacctttggtgctggtaccaaattggaactgaagtaatgactcgag

SEQ ID NO: 30 - protein, 269 aa, Artificial sequence
  Synthesized CENscFv003
MKQSTIALALLPLLFTPVTKAECQLVESGGGLVQPKGTLKLSCAASGFSFNTHAMNWVRQAPGKSLEWVARTMSKSNNYATYYADSVKDRFIISRDDSQSMLYLQMNNLKTEDTAMYYCVRDDPKRGMDYWGQGTSVTVSSGGGGSGGGGSGGGGSGIVMSQSPSSLAVSVGEKVTMSCKPSQSLLYSRNQKIYLAWYQQKPGQSPKLLIYWASTRESGVPDRFTGSGSGTDFTLIISSVRAEDLAVYYCQQYYNYPLTFGAGTKLELK

SEQ ID NO: 31 - protein, 237 aa, Artificial sequence
  Synthesized M53-HC (CEN-AB-010-HC)
MLSGLKWVFFVVFYQGVHCEVQLVESGGGLVQPKGTLKLSCAASGFSFNTHAMNWVRQAPGKSLEWVARTMSKSNNYATYYADSVKDRFIIRADDSQSMLYLQMNNLKTEDTAMYYCVRDDPKRGMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIE

SEQ ID NO: 32 - protein, 237 aa, Artificial sequence
  Synthesized M53-LC (CEN-AB-010-LC)
MDSQAQVLMLLLLWVSGTCGGIVMSQSPSSLAVSVGEKVTMSCKPSQSLLYSRNQKIYLAWYQQKPGQSPKLLIYWASTRESGVPDRFTGSGSGTDFTLIISSVRAEDLAVYYCQQYYNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNR

SEQ ID NO: 33 - protein, 8 aa, Artificial sequence
  Synthesized M53-HC CDR1
SFNTHAMN

SEQ ID NO: 34 - protein, 16 aa, Artificial sequence
  Synthesized M53-HC CDR2
TMSKSNNYATYYADSV

SEQ ID NO: 35 - protein, 8 aa, Artificial sequence
  Synthesized M53-HC CDR3
VRDDPKRQ

SEQ ID NO: 36 - protein, 16 aa, Artificial sequence
  Synthesized M53-LC CDR1
KPSQSLLYSRNQKIYL

SEQ ID NO: 37 - protein, 7 aa, Artificial sequence
  Synthesized M53-LC CDR2
WASTRES

SEQ ID NO: 38 - protein, 11 aa, Artificial sequence [sequence omitted]

(19) United States
(12) Patent Application Publication
(10) Pub. No.: US 2014/0079722 A1
(43) Pub. Date: Mar. 20, 2014

(54) EXTRACELLULAR TARGETED DRUG CONJUGATES

(71) Applicant: James R. Prudent, Madison, WI (US)

(72) Inventor: James R. Prudent, Madison, WI (US)

(73) Assignee: CENTROSE, LLC, Madison, WI (US)

(21) Appl. No.: 14/004,036

(22) PCT Filed: Mar. 9, 2012
(86) PCT No.: PCT/US12/28585

§ 371 Date: Dec. 3, 2013

Related U.S. Application Data
(60) Provisional application No. 61/450,795, filed on Mar. 9, 2011.
(60) Provisional application No. 61/500,756, filed on Jun. 24, 2011.
(60) Provisional application No. 61/507,882, filed on Jul. 14, 2011.
(60) Provisional application No. 61/551,287, filed on Oct. 25, 2011.

(51) Int. Cl. A61K 47/48; A61K 31/7068

(52) U.S. Cl. CPC A61K 47/48569; A61K 31/7068
USPC 424/181.1; 530/391.9

(57) ABSTRACT

Antibodies targeting the dysadherin subunit of the human Na,K-ATPase signaling complex that are covalently linked via a stable linker to steroid drugs that bind the alpha subunit of that complex are useful in the treatment of cancer.

### BACKGROUND OF THE INVENTION

1. Field of the Invention

The present invention provides pharmaceutical formulations and methods for treating cancer with antibody drug conjugates in which an antibody for the dysadherin subunit of the Na,K-ATPase signaling complex is linked by a stable linker to a drug that binds the alpha subunit of the Na,K-ATPase signaling complex. The invention relates to the fields of biology, chemistry, medicinal chemistry, medicine, molecular biology, and pharmacology.

2. Description of Related Disclosures

All fundamental biological processes, including development, immunity, and tumorigenesis, are related to the selective and differential expression of genes in different tissues and cell types. For example, the formation of many malignant tumors has been shown to be associated with the production and/or expression of certain specific cell surface signaling molecules. One of the goals of modern molecular medicine is to find ways to target drugs selectively to reduce or eliminate the drug's off target toxic effects. Delivering drugs to a specific target that is unique to or expressed at higher levels in diseased cells types using targeting moieties such as antibodies, peptides or aptamers has been tried. Attaching these targeting moieties directly to the drug through linkers or to nanoparticles has also been tried.

One such drug targeting system is termed, “antibody drug conjugates” or ADC for short has been studied intensively since 1985 (see, for example, U.S. patent publication No. 2009/0220529, incorporated herein by reference). Members of this class of targeted therapeutics are composed of an antibody specific to an antigen, a drug or drugs that act intracellularly, and a linker that connects the antibody to the drug(s). To make ADCs, a wide array of antibodies, linkers, and drugs have been combined and tested in a continuing effort to identify antibodies that specifically target certain cell types and release active drug only upon binding and internalization. Unfortunately, a number of technical difficulties have been encountered with the ADC approach, including the difficulty of finding a means to link the antibody and drug where the linker is stable in the circulatory structure but “unstable” once the ADC has bound to its target or has been internalized into the target cell.

More recently, there has been an exciting new development in the field with the advent of “extracellular targeted drug conjugates” or “EDCs” in which a targeting moiety, such as an antibody, that binds specifically to an extracellular target is linked to drug that acts on a nearby target via a linker that is stable in the circulatory system. EDCs differ from ADCs in that drug release is not required for efficacy; instead, the greatest efficacy is achieved when an intact EDC is bound to both the target of the targeting moiety and the target of the drug. One illustrative EDC is an EDC in which the targeting moiety targets dysadherin and the drug is a cardiac glycoside; in this EDC the targeting moiety and drug target different subunits of the Na,K-ATPase signaling complex. See PCT Pub. No. 2011/031870, incorporated herein by reference.

There remains a need for new EDCs targeting the Na,K-ATPase, new formulations containing them, methods for making them, and methods for using them alone and in combination with other drugs to treat cancer. The present invention meets these needs.

### SUMMARY OF THE INVENTION

In various embodiments, the present invention relates to EDCs composed of an antibody that binds to the dysadherin subunit of the Na,K-ATPase signaling complex covalently bound to a linker that is stable in the circulatory system that is itself covalently linked to a drug that binds to the alpha subunit of the Na,K-ATPase signaling complex. For convenience, EDCs of this class are referred to herein as “Class 1 EDCs”.

In a first aspect, the invention provides methods of treating a patient with cancer that comprise administering a therapeutically effective dose of a Class 1 EDC to a patient in need of treatment. In some embodiments, one or more drugs in addition to the Class 1 EDC is administered to the patient to treat the cancer. In various embodiments, the other drug is selected from the group consisting of gemcitabine, TRAIL (also known as tissue necrosis factor (TNF)-related apoptosis-inducing ligand and as Apo2L), fibroblast growth factor receptor kinase inhibitors, mTOR inhibitors and glycolysis inhibitors.

In a first embodiment, the patient is a lung cancer patient. In one embodiment, the lung cancer is a non-small cell lung cancer (NSCLC). In one embodiment, the lung cancer is a squamous cell carcinoma. In another embodiment, the lung cancer is a large cell carcinoma. In one embodiment, the patient is administered another drug approved for the treatment of lung cancer in combination with the Class 1 EDC. In various embodiments, the other drug is selected from the group consisting of pemetrexed, docetaxel, gefitinib, gemcitabine, vinorelbine, porfimer sodium, erlotinib, etoposide, topotecan, methotrexate, bevacizumab, carboplatin, cisplatin, and crizotinib.

In a second embodiment, the patient is a pancreatic cancer (PaCa) patient. In one embodiment, the patient is administered another drug approved for the treatment of lung cancer in combination with the Class 1 EDC. In various embodiments, the other drug is gemcitabine. In other embodiments, the other drug is selected from the group consisting of fluorouracil, erlotinib, gemcitabine, sunitinib, everolimus and Mitomycin C.

In a third embodiment, the patient is a lymphoma cancer patient. In one embodiment, the lymphoma is a B-cell lymphoma. In one embodiment, the patient is administered another drug approved for the treatment of lymphoma in combination with the Class 1 EDC. In various embodiments, the other drug is selected from the group consisting of methotrexate, doxorubicin, chlorambucil, nelarabine, bendamustine, bleomycin, bortezomib, cyclophosphamide, ibritumomab tiuxetan, procarbazine, plerixafor, pralatrexate, denileukin diftitox, ofatumumab, rituximab, romidepsin, tositumomab, vinblastine, bortezomib, vinblastine, vorinostat, interferon, romidepsin. brentuximab vedotin and britumomab tiuxetan.

In these and other embodiments of the treatment methods of the invention, the therapeutically effective dose is in the range of about 0.1 mg per kg patent weight (“mg/kg”) to about 100 mg/kg. In various embodiments, the therapeutically effective dose is from about 0.1 mg/kg to about 10 mg/kg. In various embodiments, the therapeutically effective dose is from 0.25 mg/kg to 5 mg/kg. In various embodiments, the therapeutically effective dose is administered once per week or once every three weeks, and dosing is continued at that frequency until the patient is cured or the cancer progresses.

In a second aspect, the present invention provides new Class 1 EDCs. While the Class 1 EDCs described in PCT Pub. No. 2011/031870 are suitable for use in the methods and formulations of the invention, the new invention provides a variety of new Class 1 EDCs. Generally, a Class 1 EDC is composed of (i) an antibody that binds dysadherin covalently linked to; (ii) a polyethylene glycol (PEG)-amino-glycoside linker; and (iii) a steroid drug attached to the glycoside in the linker.

In various embodiments, the number of drugs attached to each antibody (referred to as “drug loading”) of a Class 1 EDC of the invention ranges from about 2 to about 8. In one embodiment, the drug loading is 3. In another embodiment, the drug loading is 7.

In various embodiments, the drug attached to the antibody is a steroid that binds to the alpha subunit of the Na,K-ATPase signaling complex. In various embodiments, the steroid is digitoxigenin or scillarenin.

In various embodiments, the drug is attached to the linker via the C1 hydroxyl group of the glycoside, and the glycoside is selected from the group consisting of 4-amino-riboside and 4-amino-xyloside, and the PEG portion of the linker is attached to the amino group of the glycoside. In various embodiments of the Class 1 EDCs of the invention, the steroid is digitoxigenin or scillarenin, and the glycoside is either 4-amino-riboside or 4-amino-xyloside.

In various embodiments, the PEG portion of the linker contains from 2 to 36 glycol units. In various embodiments, the PEG portion of the linker contains 24 glycol units.

In various embodiments, the antibody is an M53 monoclonal antibody. In various embodiments, the antibody is a monoclonal antibody that binds to the same epitope as the M53 antibody. In various embodiments, the antibody is a humanized form of the M53 antibody.

In a third aspect, the present invention provides a pharmaceutical formulation of a Class 1 EDC suitable for parenteral, including but not limited to intravenous, administration. In one embodiment, the invention provides pharmaceutical formulations suitable for parenteral administration that comprise a Class 1 EDC in combination with a pharmaceutically acceptable vehicle, vector, diluent, and/or excipient. The present invention also provides unit dose forms of these pharmaceutical formulations. In one embodiment, the invention provides a unit dose form containing a pharmaceutical formulation of the invention suitable for intravenous administration that contains from about 5 mg to about 5 g of a Class 1 EDC. In various embodiments, these unit dose forms contain 0.5 g, 1 g, 2.5 g, or 5 g of a Class 1 EDC.

The pharmaceutical formulations of the invention can be used in vivo for preventive, ameliorative, and/or curative purposes for diseases or disorders cellular hyperproliferation. Non-limiting examples of diseases or disorders for which the pharmaceutical formulations according to the invention may be used include cancers, metastases, cellular apoptosis disorders, degenerative diseases, tissue ischemia, inflammation disorders, diabetes and pathological neo-angiogenesis. In various embodiments, the pharmaceutical formulations of the invention are used to treat cancer, including but not limited to lung cancer, lymphoma cancer, and pancreatic cancer, as noted above. Thus, in accordance with the methods of the invention, a subject can be treated with a pharmaceutically effective amount of a compound or composition according to the invention. In one embodiment of the invention, the subject is a human subject.

### BRIEF DESCRIPTION OF THE FIGURES

FIG. 1 shows the pharmacokinetics of antibody M53 and Class 1 EDC with varying drug loading as described in Example 5. “EDC-ONE” refers to the EDC with varying drug loading (either 2 (“2x”), 5 (“5X”), or 9 (“9X”).

FIG. 2 shows a body weight determination of mice dosed with a single bolus injection of different amounts of a Class 1 EDC with a drug loading of two for a post injection period of 24 days as described in Example 6.

FIGS. 3 and 4 shows the results obtained with M53-PEG24-CEN09-106 in A549 athymic nude mouse xenograft models as described in Example 7.

FIG. 5 shows the mean tumor volume and the mean body weight of mice treated with M53-PEG24-CEN09-106 in an H460 athymic nude mouse xenograft model as described in Example 7.

FIGS. 6 and 7 show the results obtained with M53-PEG24-CEN09-106 in PANC-1 xenograft models as described in Example 8.

### DETAILED DESCRIPTION OF THE INVENTION

The present invention relates to pharmaceutical formulations and unit dose forms of Class 1 EDCs and methods for using them alone and in combination with other agents in the treatment of cancer, particularly lung and pancreatic cancer. For the convenience of the reader, this detailed description of the invention is divided into sections. Section I provides definitions of terms used herein. Section II describes Class 1 EDCs provided by and useful in the methods of the invention. Section III describes methods of the invention for using Class 1 EDCs, alone and in combination with other drugs, to treat cancer. Section IV describes pharmaceutical formulations and unit dose forms of the inventions. The detailed description of the invention is followed by a set of examples that illustrate various aspects and embodiments of the invention. All patents, patent applications, and scientific literature references cited herein are incorporated herein by reference in their entireties.

### I. Definitions

The term “amino acid” refers to naturally occurring and non-natural amino acids, as well as amino acid analogs and amino acid mimetics.

The term “antibody” refers to a protein or mixture of proteins that comprise one or more peptidic chains encoded by immunoglobulin genes or fragments thereof that specifically bind and recognize an epitope of an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. The antibodies comprise IgG (including IgG1, IgG₂, IgG₃, and IgG₄), IgA (including IgA1 and IgA₂), IgD, IgE, or IgM, and IgY. As used herein, the term “antibody” is meant to include whole antibodies, including single-chain antibodies, and antigen-binding fragments thereof. Antibodies can also be antigen binding antibody fragments and include, but are not limited to, Fab, Fab′ and F(ab′)₂, Fd, single-chain Fvs (scFv), single-chain antibodies, disulfide-linked Fvs (sdFv), diabodies, triabodies, tetrabodies, minibodies, and fragments comprising either a V_{L} or V_{H} domain, and Nanobodies (see PCT publication number WO 94/04678 and Nature Medicine, V9 (1) pp 129-134, 2003). An antibody can be from any animal origin including birds and mammals. Typically, antibodies in commercial or research use are human, murine, rabbit, goat, guinea pig, camelidae (e.g., camel, llamas), horse, or chicken antibodies. “Antibodies”, as used herein, includes monoclonal, chimeric, and humanized antibodies, as well as intact antibodies and isolated antibodies. Antibodies can be monospecific, bispecific, trispecific or greater multispecificity.

The term “extracellular-targeted drug conjugate” or “EDC” refers to a drug conjugate in which an antibody or other targeting moiety that targets an extracellular target is linked via a stable or non-cleavable linker to a drug that binds to an extracellular target.

The term “antigen” refers to the substance or target that an antibody or targeting moiety binds. An antigen is characterized by its ability to be “bound” by the antibody or targeting moiety. Antigen can also mean the substance used to elicit the production of targeting moieties, such as the production of antigen specific antibodies through immunizing with the antigen.

The term “antigen binding site” or “epitope” refers to the portion of the antigen to which an antibody binds.

The term “binding affinity” refers to the strength of interaction between an antibody (or other targeting moiety or drug or other agent) and its antigen (or target) as a function of its association and dissociation constants. Higher affinities typically mean that the targeting moiety has a fast on rate (association) and a slow off rate (dissociation). Binding affinities can change under various physiological conditions and changes that occur to the antigen or antibody/targeting moiety under those conditions. Binding affinities of the targeting moiety can also change when therapeutic agents and/or linkers are attached. Binding affinities can also change when slight changes occur to the antigen, such as changes in the amino acid or glycosylation of the antigen.

The term “cancer” refers to any of a number of diseases characterized by uncontrolled, abnormal proliferation of cells, the ability of affected cells to spread locally or through the bloodstream and lymphatic system to other parts of the body (i.e., metastasize), as well as any of a number of characteristic structural and/or molecular features. A “cancerous cell” or “cancer cell” is understood as a cell having specific structural properties, which can lack differentiation and be capable of invasion and metastasis. Examples of cancers are, breast, lung, brain, bone, liver, kidney, colon, and prostate cancer (see DeVita, V. et al. (eds.), 2005, Cancer Principles and Practice of Oncology, 6th. Ed., Lippincott Williams & Wilkins, Philadelphia, Pa., incorporated herein by reference in its entirety for all purposes).

The term “chimeric antibodies” refers to antibodies in which the Fc constant region of a monoclonal antibody from one species (typically a mouse) is replaced, using recombinant DNA techniques, with an Fc region from an antibody of another species (typically a human). For example, a cDNA encoding a murine monoclonal antibody is digested with a restriction enzyme selected specifically to remove the sequence encoding the Fc constant region, and the equivalent portion of a cDNA encoding a human Fc constant region is substituted. A CDR-grafted antibody is an antibody in which at least one CDR of a so-called “acceptor” antibody is replaced by a CDR “graft” from a so-called “donor” antibody possessing desirable antigen specificity. Generally the donor and acceptor antibodies are monoclonal antibodies from different species; typically the acceptor antibody is a human antibody (to minimize its antigenicity in a human), in which case the resulting CDR-grafted antibody is termed a “humanized” antibody. The graft may be of a single CDR (or even a portion of a single CDR) within a single V_{H} or V_{L} of the acceptor antibody, or can be of multiple CDRs (or portions thereof) within one or both of the V_{H} and V_{L}. Methods for generating CDR-grafted and humanized antibodies are taught by Queen et al. U.S. Pat. No. 5,585,089, U.S. Pat. No. 5,693,761 and U.S. Pat. No. 5,693,762; and Winter U.S. Pat. No. 5,225,539, which are incorporated herein by reference.

The term “circulatory structure” refers to body fluids, interstitial fluid, lymph and blood of a mammal, including tissues of the circulatory system.

The terms “dysadherin”, “ATPase subunit gamma 5”, “FXYDS”, or “gamma 5” are used interchangeably herein and refer to the gamma subunit 5 of the Na,K-ATPase signaling complex.

The term “epitope” refers to groupings of molecules such as amino acid residues or sugar side chains at the surface of antigens that usually have specific three dimensional structural characteristics, as well as specific charge characteristics, and that are capable of specific binding by a monoclonal antibody.

The term “extracellular” refers to the outer surface of a cell membrane.

The term “intact antibody” comprises at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR^{X} or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. Examples of binding fragments include (i) a Fab fragment, a monovalent fragment consisting of the V_{L}, V_{H}, CL and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarily determining region (CDR).

The term “hetereobifunctional linker” refers to a linker with different reactive groups at either end, enabling sequential conjugation between two different functional groups in proteins and other molecules.

The term “extracellular target” refers to a target, such as a protein, antigen, and/or epitope located on the outer surface of the cell membrane.

The term “linker” refers to a chemical moiety or bond that covalently attaches two or more molecules, such as a targeting moiety and a drug.

The term “linker spacer group” refers to atoms in the linker that provide space between the two molecules joined by the linker.

The term “monoclonal antibody” refers to a preparation of antibody molecules of single molecular composition. A monoclonal antibody composition displays a single binding specificity and affinity for a particular epitope. The term “human monoclonal antibody” refers to antibodies displaying a single binding specificity which have variable and constant regions (if present) derived from human germline immunoglobulin sequences. Human monoclonal antibodies can be produced by a hybridoma which includes a B cell obtained from a transgenic non-human animal, e.g., a transgenic mouse, having a genome comprising a human heavy chain transgene and a light chain transgene fused to an immortalized cell, although the term “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technology.

The term “modified antibodies” refers to antibodies, such as monoclonal antibodies, chimeric antibodies, and humanized antibodies, which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Multiple molecules of a therapeutic agent or multiple different agents can be coupled to one antibody molecule. For example, different moieties can be coupled to an antibody molecule via the same linker, or multiple linkers that provide multiple sites for attachment (e.g., dendrimers) can be used.

The terms “non-cleaved” and “uncleaved” refer to an EDC composition at any point in time in which the majority (for example, >50%, >60%, >70% or >80%) of EDC components present are intact, i.e., the linker used to attach the agent to the targeting moieties has not been cleaved.

The term “non-cleavable linker” refers to a stable linker that has the property of being more stable in vivo than either the therapeutic or the targeting moiety under the same physiological conditions. Examples of non-cleavable linkers include linkers that contain polyethylene glycol chains or polyethylene chains that are not acid or base sensitive (such as hydrazone containing linkers), are not sensitive to reducing or oxidizing agents (such as those containing disulfide linkages), and are not sensitive to enzymes that may be found in cells or circulatory system.

The terms “pharmaceutically effective amount” and “effective amount” in the context of an amount of drug delivered refer to an amount of a drug that can induce a desired biological or medical response in a tissue, system, animal, or human.

The terms “peptide”, “polypeptide”, peptidomimetic and “protein” are used, somewhat interchangeably, to refer to a polymer of amino acid residues. The terms apply to amino acid polymers in which one or more amino acid residues is an artificial chemical mimetic of a corresponding naturally occurring amino acid, as well as to naturally occurring amino acid polymers. These terms also encompass the term “antibody”. “Peptide” is often used to refer to polymers of fewer amino acid residues than “polypeptides” or “proteins”. A protein can contain two or more polypeptides, which may be the same or different from one another.

The term “receptor” refers to an extracellular target protein molecule, embedded in either the plasma membrane or the cytoplasm of a cell, to which one or more specific kinds of signaling molecules may bind. Each cell typically has many receptors, of many different kinds.

The term “substantially simultaneously” refers to two or more events that occur at the same time or within a relatively narrow time frame. In various embodiments, substantially simultaneously refers to two or more events that occur within about 60, about 40, about 30, about 20, about 10, about 5, about 2 or about 1 second of each other. For example, EDCs of the invention have properties such that targeting moiety binding and agent (drug) action happen substantially simultaneously.

The term “stable in the circulatory structure” refers to the property of a compound, such as an EDC, to resist degradation and means that, for example, less than about 50%, or less than about 20%, or typically less than about 2%, of the compound is degraded or cleaved in the circulating blood at about 37° C. for at least about 2 hours.

The term “stable linker” refers to a linker that remains stable and intact until the conjugate has been delivered or transported to the target site—a stable linker remains covalently attached to the two molecules it links—in physiological conditions (at 37° C. and pH 7) in vivo or in vitro for a period of time sufficient to allow the EDC to reach the target(s) and bind to the target(s). Thus, a stable linker is generally stable within the circulatory structure (generally means below 5% degradation after at least a 2 hour period and, in some embodiments, at least 4, 8, 16, or 24 hour periods).

The term “synergistically” refers to an effect of two or more agents when used in combination that is greater than the sum of the effects of both agents when used alone. For example, in the EDCs of the invention, the combined therapeutic effects of the interaction of the antibody and the agent (drug) when linked through a linker are greater than the combined individual effects of the targeting moiety and agent when used alone. “Effects” can refer either to binding, therapeutic effect, and/or specificity.

The term “target” refers to the protein, glycoprotein, antigen, carbohydrate or nucleic acid to which a targeting moiety binds and also refers to the protein, glycoprotein, antigen, carbohydrate or nucleic acid to which a therapeutic agent (which may be referred to herein as a “drug”) binds. The agent and targeting moiety may bind to different targets in a “target complex”, where “target complex” refers to two or more molecules, such as the different subunits of a multi-subunit protein or two different proteins in a multi-protein complex, that are in close physical proximity with one another in vivo.

The term “target cells” refers to the cells that are involved in a pathology and so are preferred targets for therapeutic activity. Target cells can be, for example and without limitation, one or more of the cells of the following groups: primary or secondary tumor cells (the metastases), stromal cells of primary or secondary tumors, neoangiogenic endothelial cells of tumors or tumor metastases, macrophages, monocytes, polymorphonuclear leukocytes and lymphocytes, and polynuclear agents infiltrating the tumors and the tumor metastases.

The interchangeable terms “targeting moiety” and “targeting agent” refer to an antibody that binds specifically to a target.

The term “target tissue” refers to target cells (e.g., tumor cells) and cells in the environment of the target cells.

The terms “therapeutic agent” and “drug” and “agent” are used interchangeably herein to refer to a compound that, when present in a therapeutically effective amount, upon binding to a site of action, produces a therapeutic effect, and whose site of action is located or whose effect will be exerted on the surface or inside target cells.

The term “therapeutic effect” refers to the reduction, elimination, and/or prevention of a disease, symptoms of the disease, or side effects of a disease in a subject.

The term “to increase the half-life” means to increase the mean residence time of a compound, typically a therapeutic agent, in the blood or to reduce the blood or plasmatic clearance compared to a reference compound.

The terms “treating” and “treatment” are used interchangeably to refer to the administration of a therapeutic agent or composition to a patient who has a disease or disorder (e.g., cancer or metastatic cancer), a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease. “Treating” or “treatment” of cancer or metastatic cancer refers to the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters, including the results of an examination by a physician. Accordingly, the term “treating” includes the administration of a therapeutic agent to prevent or delay, to alleviate, or to arrest or inhibit development of the symptoms or conditions associated with a disease, including but not limited to neoplastic disease.

The term “tumor specific antigen” refers to proteins or other molecules that are unique to a tumor or is at least more abundant on tumor cells, relative to normal cells.

### II. Class 1 EDCs

Class 1 EDCs comprise an antibody that targets or binds to human dysadherin linked via a stable or non-cleavable linker (the linker is intact, non-cleaved, when the EDC exerts its maximal therapeutic effect) to a steroid drug that binds the alpha subunit of the Na,K-ATPase signaling complex. Thus, in all embodiments, a Class 1 EDC contains a targeting moiety that binds to an extracellular target, and while attached to a steroid via a stable or non-cleavable linker, exerts a therapeutic effect by acting on a human Na,K-ATPase signaling complex. Each of these components is discussed below, after a brief description of the Na,K-ATPase signaling complex.

### A. Na,K-ATPase Signaling Complex

The human Na,K-ATPase signaling complex has multiple subunits, isoforms and/or glycosylation patterns that determine its presence and, if present, location in or on cells. Presentation can depend on cell type, location on the cell, location of the cell, and/or physiological and pathological conditions. For example, the type of beta subunit (1 vs. 2) found in the Na/K-ATPase signaling complex and its glycosylation pattern differ from cell type to cell type (see Proteomics 2008; 8(16):3236-56, and Am J Physiol 1997; 272(1 Pt 1):L85-94). Aberrant glycosylation is a hallmark of cancer and includes alterations in the carbohydrate content of glycoproteins, glycolipids, and glycosaminoglycans (see Anticancer Agents Med Chem 2008; 8(1):2-21 and Biochim Biophys Acta 1999; 1473(1):21-34). “Dysadherin”, also referred to as gamma subunit isoform 5 of the Na/K-ATPase signaling complex or as “FXYD5”, as it is encoded by the FXYD5 gene, is a glycosylated membrane protein that has been shown to promote experimental cancer metastasis and is an independent prognostic indicator of metastasis and survival for many different types of human cancer [see Nam et. al. Cancer Lett. 255(2) 161-9 (2007)].

The Class 1 EDCs of the invention comprises a steroid drug, a stable or non-cleavable linker, and an antibody that recognizes a cell surface-exposed epitope of the dysadherin subunit of the Na,K-ATPase signalling complex. The Na,K-ATPase is characterized by a complex molecular heterogeneity that results from the expression and differential association of multiple isoforms of its alpha-, beta- and gamma-subunits (see review in Am. J. Physiol. 275 (Renal Physiol. 44): F633-F650, 1998). The Na,K-ATPase belongs to a widely distributed class of P-type ATPases that are responsible for the active transport of a variety of cations across cell membranes. At present, as many as four different alpha-isoforms, three distinct beta-isoforms, and nine distinct gamma-isoforms have been identified in mammalian cells. The stringent constraints on the structure of the complex's isoforms during evolution and their tissue specific and developmental pattern of expression suggests that different Na,K-ATPase complexes have evolved distinct properties to respond to cellular requirements. Different isoforms of the alpha-subunit are expressed at different levels on different cell types and behave differently. The alpha-subunit contains the binding sites for cations, ATP, Src kinase, and various therapeutic agents, including steroid drugs contained in the cardiac glycoside class of molecules. Therefore in one embodiment of the invention, the alpha-subunit can act as the target for the agent of EDCs of the invention and the agent is a steroid drug of a cardiac glycoside. Specifically, the cardiac glycoside class of molecules has been mainly used therapeutically in the treatment of cardiac failure, due to their anti-arrhythmic effects. Recently it was determined that this class of drugs also has anti-cancer activities, yet use as an anti-cancer drug has not yet been approved due to cardiotoxicity at levels required. Targeting this class of molecules away from the heart and toward cancer cells would thus be beneficial.

The beta-subunit of the Na,K-ATPase complex is believed to act as a chaperone for the alpha-subunit, directing its location on the cell membrane and can be aberrantly glycosylated on certain diseased cells.

The gamma-subunit's specific role is thought to regulate the activity of ion transport and has been shown to modify voltage dependence of the complex. The gamma-subunit is thought not to be required for ATPase activity (Biochem Biophys Res Commun 1981 102:250-257). Specifically, the gamma subunit isoform 5 is over-expressed on certain cancer cell types and appears to be a sole prognosticator of metastasis (Nam, J. et al. Cencer Lett. 255(2): 161-169). Gamma-subunits are constructed from a FXYD peptide span that is universal. There are multiple FXYD or gamma-subunit isoforms and expression differs by cell type and cell environment. This subunit also has been shown to complex with other proteins besides the Na,K-ATPase ion pump. Tissue/cell-specific expression of the regulatory FXYD subunits of Na-K-ATPase is not static, and may be changed to adapt to a given physiological or pathological situation. It is believed that a complex that includes an FXYD subunit will do so based on expression levels of the various isoforms and competition with the complexes it associates with. Therefore, expression of FXYD and specifically FXYD5, does not always indicate that it will be associated with the Na,K-ATPase ion channel. In the Class 1 EDCs of the invention, gamma-subunit 5 (also known as dysadherin and FXYD5) is the target for the antibody of the EDCs. In particular, an EDC of the invention comprises a steroid drug that acts on the alpha subunit of the Na,K-ATPase signaling complex, a non-cleavable linker, and an antibody which binds to the gamma 5 subunit FXYD5. In various embodiments, the antibody is M53 or another antibody that has a variable sequence identical to a heavy or light chain variable sequence of M53 or another antibody that binds to the same epitope as M53 (see Example 2, below). In various embodiments of the invention, the EDC comprises scillarenin, scillarenin-4-amino-4-deoxy-L-xylopyranoside, and PEG24-CEN-09-106.

### B. Antibody Component of Class 1 EDC

Antibodies have been generated to dysadherin, including the monoclonal antibody NCC-M53 (M53) [Shimamura et al. J. Clinical Oncology 21(4) 659-667 (2003)], and these antibodies can be used in the Class 1 EDCs of the invention. Moreover, as described in Example 2 below, the epitope on dysadherin recognized by the M53 antibody has been mapped, and any antibody that binds this epitope specifically and binds to dysadherin on the surface of tumor cells can be used as the antibody in a Class 1 EDC of the invention.

Thus, the antibody in a Class 1 EDC specifically binds to an extracellular domain of human dysadherin. The antibody of a Class 1 EDC may comprise a heavy and/or light chain of M53 (CEN-AB-010; SEQ ID NOS: 31 and 32, respectively). In one embodiment, the antibody of a Class 1 EDC may comprise one or more CDRs from the heavy chain of M53 (e.g., SEQ ID NOS: 33-35). In one embodiment, the antibody of a Class 1 EDC may comprise one or more CDRs from the light chain of M53 (e.g., SEQ ID NOS: 36-38). In one embodiment, the antibody of a Class 1 EDC may comprise one or more CDRs from the heavy chain of M53 (e.g., SEQ ID NOS: 33-35) and one or more CDRs from the light chain of M53 (e.g., SEQ ID NOS: 36-38).

The antibody of the Class 1 EDC may be a monoclonal antibody, e.g. a murine monoclonal antibody, a chimeric antibody, a human antibody or a humanized antibody. In one embodiment the antibody is a humanized antibody, for example, a humanized form of M53. In one embodiment the antibody is the chimeric form of M53 described in Example 4, below. In one embodiment, the antibody is an antibody fragment, e.g. a Fab fragment. In one embodiment, the antibody of the Class 1 EDC binds to or selectively binds to an epitope within the polypeptide represented by SEQ ID NO: 1. In one embodiment, the antibody of the Class 1 EDC binds to the epitope on FXYD5 recognized by the M53 antibody.

Various methods previously employed to produce monoclonal antibodies (MAbs) can be used to produce antibodies for use in Class 1 EDCs. Hybridoma technology, which refers to a cloned cell line that produces a single type of antibody, uses the cells of various species, including mice (murine), hamsters, rats, and humans. Other methods to prepare MAbs, including chimeric and humanized antibodies, employ genetic engineering, i.e. recombinant DNA techniques. Monoclonal antibodies are obtained from a population of substantially homogeneous antibodies. Human myeloma and mouse-human heteromyeloma cell lines also have been described for the production of human monoclonal antibodies (Kozbor, (1984) J. Immunol., 133:3001, and Brodeur et al., Monoclonal Antibody Production Techniques and Applications, pp. 51-63 (Marcel Dekker, Inc., New York, 1987)).

DNA encoding the monoclonal antibodies is readily isolated and sequenced; hybridoma cells serve as a source of such DNA. Once isolated, the DNA is placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies (see US 2005/0048572; US 2004/0229310; Skerra et al (1993) Curr. Opinion in Immunol. 5:256-262; and Pluckthun (1992) Immunol. Revs. 130:151-188. The DNA also may be modified, for example, by substituting the coding sequence for human heavy chain and light chain constant domains in place of the homologous murine sequences (see U.S. Pat. No. 4,816,567 and Morrison et al (1984) Proc. Natl. Acad. Sci. USA 81:6851), or by covalently joining to the immunoglobulin coding sequence all or part of the coding sequence for a non-immunoglobulin polypeptide.

As an alternative to humanization, human antibodies can be generated. Transgenic animals (e.g., mice) are capable, upon immunization, of producing a full repertoire of human antibodies in the absence of endogenous immunoglobulin production (Jakobovits et al (1993) Proc. Natl. Acad. Sci. USA, 90:2551; Jakobovits et al (1993) Nature 362:255-258; Bruggermann et al (1993) Year in Immuno. 7:33; U.S. Pat. No. 5,591,669; U.S. Pat. No. 5,589,369; and U.S. Pat. No. 5,545,807).

Antibody fragments can be obtained by proteolytic digestion of intact antibodies (see Morimoto et al (1992) J. Biochem. Biophys. Meth. 24:107-117; and Brennan et al (1985) Science 229:81) or produced directly by recombinant host cells. Fab′-SH fragments can be directly recovered from E. coli and chemically coupled to form F(ab′)₂ fragments (Carter et al (1992) Bio/Technology 10:163-167). F(ab′)₂ fragments can be isolated directly from recombinant host cell culture. Single chain Fv fragments (scFv) can be prepared as described in PCT Pub. No. WO 93/16185; U.S. Pat. No. 5,571,894; and U.S. Pat. No. 5,587,458. An antibody fragment may also be a “linear antibody”, e.g., as described in U.S. Pat. No. 5,641,870, for example.

Antibodies with more than two valencies can be employed in various embodiments of the EDCs of the invention. Multivalent, “Octopus” antibodies with three or more antigen binding sites and two or more variable domains can be readily produced by recombinant expression of nucleic acid encoding the polypeptide chains of the antibody (US 2002/0004586; WO 01/77342). For example, trispecific antibodies can be prepared (Tutt et al (1991) J. Immunol. 147:60).

The antibodies in the EDCs of the invention can include various antibodies within the scope of the descriptions provided above. Thus, the antibody in a Class 1 EDC can be a fully humanized antibody, a human chimera or an antibody fragment, including antigen binding Fabs, Fvs, scFv, and minibodies, and the antibody can also be enhanced to increase the antibody's affinity, stability, and expression level (see Nat. Med. 2003 January; 9(1):129-34).

Typically, the antibody will be purified to greater than 95% by weight (as determined, for example, by the Lowry method), and often to more than 99% by weight prior to use in forming a Class 1 EDC. Ordinarily, the antibody will be prepared by at least one purification step. Once an antibody is available in sufficient purity, it can be linked to a therapeutic agent by any of a variety of linkers and linking chemistries, as discussed herein.

### C. Linker Component of Class 1 EDC

To form a Class 1 EDC, a steroid drug is coupled to the dysadherin antibody via a stable linker. The linker, if conceptualized as a discrete entity instead of part of a Class 1 EDC, is a bifunctional or multifunctional moiety that can be used to link one or more drugs to an antibody to form an EDC. EDCs can be conveniently prepared using a linker having reactive functionality for binding to the drug and to the antibody. For example, a cysteine thiol, or an amine, e.g. N-terminus or amino acid side chain such as lysine, of an antibody can form a bond with a functional group of a linker reagent or drug-linker reagent.

Linkers for use in the Class 1 EDCs preferred for the methods of the present invention are generally composed of polyethylene glycol (PEG) and an amino-glycoside. In various embodiments, the PEG portion of the linker contains from 2 to 36 glycol units. In various embodiments, the PEG portion of the linker contains 24 glycol units. Typically, in the manufacture of an EDC in accordance with the methods of manufacture of the invention, a drug-linker reagent is formed, and the drug-linker reagent is covalently coupled to the antibody to form the Class 1 EDC. Typically, the steroid drug is attached to the linker via the C1 hydroxyl group of the amino-glycoside, and the glycoside is selected from the group consisting of 3-amino-riboside, 4-amino-riboside, 3-amino-xyloside, and 4-amino-xyloside, and the PEG portion of the linker is attached to the amino group of the glycoside. When the steroid is digoxigenin, progesterone, or scillarenin, the glycoside is either 4-amino-riboside or 4-amino-xyloside. When the steroid is ouabain, the glycoside is 3-amino-riboside or 3-amino-xyloside.

The linkers employed in the EDCs of the invention are stable. After administration, the EDC is stable and remains intact, i.e. the targeting moiety remains linked to the agent via the linker. The linkers are stable outside the target cell and remain uncleaved for efficacy. An effective linker will: (i) maintain the specific binding properties of the antibody; (ii) allow delivery of the conjugate or agent; (iii) remain stable and intact, i.e. not cleaved, for as long as the antibody and/or agent remains stable and intact; and (iv) maintain a cytotoxic, cell-killing effect or a cytostatic effect of the agent while the EDC is intact. By way of example, stable linkers are those that, when in an EDC of the invention, show minimal (i.e., less than 10%) cleavage while present in the circulatory structure, at the surface of target tissue, at the surface of target cell, or in the extracellular matrix for a period of at least 4 to 8 hours or longer, such as 8 to 24 hours, or 1 to 10 days or longer; non-cleavable linkers are stable in these conditions for longer periods, including periods as long as 20 days or longer (Durcy, L. et. al. Bioconjugate Chem. 2010, 21, 5-13).

The linkers employed in the EDCs of the invention can be conveniently produced in two stages. In the first stage, a glycoside that contains an active nucleophile such as a free primary amine is attached. In the second stage, a bifunctional PEG linker is attached to the glycoside's amine. This method is advantageous in that it allows various combinations of glycosides and different linker lengths to be added in succession. In addition, glycosides have been shown to have certain advantages when employed in the linker portion of the invention.

A stable linker forms a covalent bond between the therapeutic agent and a targeting moiety such that, when attached, the agent and targeting moiety can bind and act on their respective targets. While a stable linker can simply be a covalent bond formed between reactive sites on the targeting moiety and the agent, the stable linkers of the invention typically include a linker spacer group, i.e., a repeating series of ethylene glycol units and an amino-glycoside. To attach a targeting moiety to an agent through a linker, one utilizes complementary reactive groups. For example, accessible sulfhydryl groups on a targeting moiety can react with active maleimide groups to form stable thioether linkages. An additional example is accessible amines on an agent can react with succinimide esters to form stable amide bonds. Bifunctional linkers which have maleimides on one end and succinimide esters on the other can be used to link the drug to the antibody. As illustrated in the examples below, a Class 1 EDC can be conveniently prepared by linking an amino glycoside to a hydroxyl group of a steroid drug forming an β-glycosidic linkage. Then an NHS-PEG-maleimide reagent is linked to the amino group of the amino glycoside to form a “linker-reagent”. Finally the maleimide in the linker-reagent is covalently attached to a cysteine moiety in the antibody.

Thus, distinct chemical linkers (as opposed to a single covalent bond) are typically used in EDCs. Linkers of this type are typically linear chains of atoms or polymers consisting of one or more “linker spacer groups” with two “ends” that contain functional groups that can serve as linking reagents to connect the targeting moiety and/or therapeutic agent to the linker covalently. Suitable linkers can include a wide variety of functional groups and moieties, including but not limited to substituted or unsubstituted alkyl, substituted or unsubstituted heteroalkyl, substituted or unsubstituted aryl, aldehydes, acids, esters and anhydrides, sulfydryl or carboxyl groups, such as maleimido benzoic acid derivatives, maleimidocaproic acid derivatives, and succinimido derivatives, or may be derived from cyano bromide or chloride, succinimidyl esters or sulphonic halides and the like.

The linker can impart beneficial properties to an EDC of the invention in addition to physically linking the targeting moiety and the drug. The linker can be used to minimize agent self-association or aggregation of the EDC caused by the agent. The linker may also improve the therapeutic efficacy of the EDC. The linker may also improve the pharmacokinetics of the EDC. When the targeting moiety is linked to the therapeutic agent, the linker group may have several other functions, such as making the compound of the invention more bio-resistant, more bio-compatible, less immunogenic, less toxic, and/or more stable while in the circulatory structure or more stable to other types of destruction or elimination or to make it non-cleavable. Thus in certain embodiments, the stable or non-cleavable linker maintains the attachment of the targeting moiety to the therapeutic agent under physiological conditions, but may also have beneficial therapeutic effects as well.

An example of a stable, non-cleavable linker is the polyalkylene glycol linker. Another example of a stable, non-cleavable linker is a glycoside attached to a polyalkylene glycol linker. Polyalkyleneglycol linkers are linear chains that have at least two, and typically more than two, alkylene moieties linked together by oxygen in the form of an ether linkage. Glycoside attached polyalkylene glycol linkers are linear chains of polyalkylene glycol that has a sugar, such as an aminoglycoside, attached. The alkylene groups can be substituted, but typically are unsubstituted, and can comprise any desired number of alkylene units, but typically at least 2 or no more than 100 such units, e.g., ethylene, propylene, hexylene, and the like. In one embodiment, the linker is composed of 24 repeating ethyleneglycol units making a PEG24-type linker. This linker would be approximately 90-100 angstoms long depending on the reactive groups attached to either end. Generally, the linker length will be in the range of about 50 to about 500 Angstroms or about 50 to about 200 Angstroms. In one embodiment, the linker is composed of a sugar. In various embodiments, as described above, the linker contains an amino sugar. The polyalkyleneglycol residue can comprise repeating alkylene units which are all the same or which vary in length and/or substitution. In various embodiments, the linker of the EDC of the invention is constructed using a (PEG)36 bifunctional linker. In a particular embodiment, the linker of the EDC of the invention is constructed using SM(PEG)24 from Thermo Scientific.

When polyethyleneglycol (PEG) is used to link the targeting moiety to the drug, the EDC may be capable of withstanding attacks by the immune system. Adding PEG to proteins or small molecules has been shown to improve therapeutic efficacy of some protein or small molecule therapeutics (see PEGylated Protein Drugs: Basic Science and Clinical; Applications Series: Milestones in Drug Therapy Veronese, Francesco M. (Ed.)2009 and Advanced Drug Delivery Reviews Volume 55, Issue 10, 26 Sep. 2003, Pages 1261-1277, incorporated herein by reference). PEG can therefore increase the serum half-life and reduce antigenicity.

When a sugar, such as an aminoglycoside, is used to link the antibody to the drug via a polyalkylene glycol, the EDC may have an enhanced ability to withstand attacks by the immune system relative to EDC lacking such a sugar. Adding sugars to proteins or small molecules has been shown to improve therapeutic efficacy of antibodies or small molecule therapeutics (see Nature Reviews Drug Discovery 8, 226-234 (March 2009) and see Essentials of Glycobiology. 2nd edition. Cold Spring Harbor Laboratory Press; 2009). Sugars can therefore can increase solubility thus reducing aggregation and reduce antigenicity.

While the order of attachment of the antibody, linker portions, and drug can be varied in the manufacture of an EDC, typically the manufacturing process proceeds by first synthesizing a drug, then attaching the sugar portion of the linker, then attaching the PEG portion of the linker and finally attaching antibody. As those of skill in the art will appreciate, an antibody may present multiple sites for covalent attachment of the drug-linker reagent (or linker). By appropriate modification of the coupling conditions, one can make preparations of EDC in which the average number of drugs per antibody varies according to the conditions employed. In various methods of the invention, this average number is important in achieving maximal beneficial therapeutic effect of the EDC, as discussed below.

### D. Steroid Drug in Class 1 EDCs

A variety of steroid drugs are suitable for use in Class 1 EDCs in accordance with the invention. For example and without limitation, the steroid drug can be an agent with anti-tumor, anti-angiogenic, or anti-inflammatory therapeutic activity. In various embodiments, the steroid drug is scillarenin. In various embodiments, the steroid drug is digitoxigenin.

Generally, the steroid component of any cardiac glycoside can be used as a steroid drug in a Class 1 EDC of the invention. Digitoxin and proscillaridin are cardiac glycosides that have strong antitumor activities but high cardiotoxicity (see, Arch Pharm Res 2007; 30:10, 1216-1224). Cardiac glycosides are a class of drugs derived from plants of the genera Digitalis, Strophanthus, and others, which have been prescribed for centuries to treat congestive heart failure and arrhythmias. In these conditions, cardiac glycosides bind to the alpha subunit Na,K-ATPase signaling complex and inhibit its pumping activity. Studies performed over the last decade show that cardiac glycosides have activity as anti-cancer agents [Mijatovic et al. (2007) Biochim Biophys Acta 1776:32-57 and PCT Pub. No. 2010/017480].

In various embodiments of the Class 1 EDCs used in the pharmaceutical formulations of the invention, the steroid is scillarenin. In various embodiments, the steroid is digitoxigenin. In various embodiments, the steroid is a compound identified in PCT Pub. No. WO 2010/017480 (PCT/US2009/053159).

Non-limiting examples of suitable steroid drugs include those of Formula I below as well as pharmaceutically acceptable esters, derivatives, conjugates, hydrates, solvates, prodrugs and salts thereof, or mixtures of any of the foregoing:

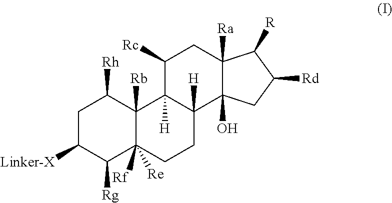

where the steroidal rings are either saturated, unsaturated or a combination thereof,

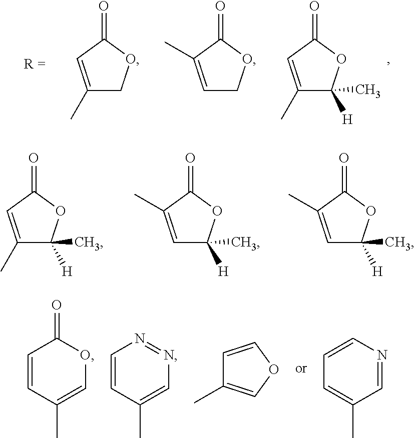

R^{a} is CH₃; R^{b} is CH₃, CH₂OH, or CHO; Rₑ is H, OH or CH₃COO; R_{d} is H, OH or CH₃COO; Rₑ is H or no group; R_{f} is H, OH or, when Rₑ is H or a C═C exists between the atoms joined to Rₑ, R_{f} and R_{g}, R_{f} is no group; R_{g} is H or, when Rₑ is H or a C═C exists between the atoms joined to Rₑ, R_{f} and R_{g}, R_{g} is no group; Rₕ is H or OH; X is O or N(OR′); and R′ is an alkyl or aryl group.

Thus, a wide variety of steroid drugs can be employed in a Class 1 EDC.

### E. Conjugation Chemistry

An EDC can be prepared by any of several routes, employing organic chemistry reactions, conditions, and reagents known to those skilled in the art, including: (1) reaction of a nucleophilic group or an electrophilic group of an antibody with a bivalent linker reagent to form an antibody-linker intermediate via covalent bonding followed by reaction with an activated drug; and (2) reaction of a nucleophilic group or an electrophilic group of a drug with a linker reagent (which may be the complete linker or a portion thereof, and if only a portion thereof, then the remaining portion may be subsequently covalently joined) to form a drug-linker intermediate (a “drug-linker reagent”) via covalent bonding followed by reaction with the nucleophilic group or an electrophilic group of an antibody. Conjugation methods (1) and (2) may be employed with a variety of antibodies, drugs, and linkers to prepare an EDC of the invention.

Nucleophilic groups on antibodies for example include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, Mass.). Each cysteine disulfide bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol.

Antibody-drug conjugates may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug (Hermanson, G. T. (1996) Bioconjugate Techniques; Academic Press: New York, p234-242). In another embodiment, proteins containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

As illustrated in the examples below, a Class 1 EDC can be conveniently prepared by linking an amino glycoside to a hydroxyl group of a steroid drug forming an β-glycosidic linkage. Then an NHS-PEG-maleimide reagent is linked to the amino group of the amino glycoside to form a “linker-reagent”. Finally the maleimide in the linker-reagent is covalently attached to a cysteine moiety in the antibody.

### F. Drug Loading

Drug loading refers to the average number of drugs per antibody in an EDC preparation. Where each linker is linked to one agent, the average number of agents will equal the average number of linkers on the antibody. Agent loading typically ranges from 1 to 8 drugs per antibody, i.e. where 1, 2, 3, 4, 5, 6, 7, or 8 agents are covalently attached to the antibody. Because there are usually multiple sites on an antibody where the linker (or drug-linker reagent) can covalently attach, and because the chemistry of attachment is difficult to direct to only a subset of the potential attachment sites, most preparations of EDC will be a mixture of antibodies having different drug loading. Thus, compositions of EDCs typically include collections of antibodies conjugated with a range of drugs, from 1 to 8.

Typically, fewer than the theoretical maximum of drug moieties are conjugated to an antibody during a conjugation reaction. An antibody may contain, for example, many cysteine residues that do not react with a drug-linker intermediate (D-L) or linker reagent. Also, only the most reactive cysteine thiol groups may react with a thiol-reactive linker reagent. Generally, antibodies do not contain many, if any, free and reactive cysteine thiol groups that may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an EDC may be controlled in several different manners, including: (i) limiting the molar excess of drug-linker (D-L; referred to in the examples below as “linker-ready therapeutic agent) or linker reagent relative to antibody, (ii) limiting the conjugation reaction time or temperature, and (iii) partial or limiting reductive conditions for cysteine thiol modification.

Drug loading can affect the pharmacodynamics, activity, toxicity, and antibody stability of an EDC. Therefore, one skilled in the art understands that it is important to optimize the number of drugs per antibody. To optimize EDC drug loading, certain EDC properties can be measured such as serum half-life and stability, and in vivo efficacy versus toxicity. From a reaction mixture, there are multiple methods described in the literature that describe how to purify antibody drug conjugates loaded with optimal number of drugs away from those antibodies that have suboptimal loading (see U.S. Pat. No. 7,811,572).

The present invention arises in part from the discovery that the drug loading for optimal therapeutic benefit can differ between cancers. Generally, however, the optimal drug loading is in the 3-6 range. In one embodiment, a Class 1 EDC in the pharmaceutical formulations of the invention has, on average, three drugs per EDC when the EDC is a monoclonal antibody. In another embodiment, a Class 1 EDC in the pharmaceutical formulations of the invention has, on average, seven drugs per EDC when the EDC is a monoclonal antibody. As illustrated in the examples below, the lower the drug loading, the longer the half-life of the EDC, and the lower the cytotoxicity of the EDCs. Thus, EDCs of the invention with a drug loading of 3, for example, can be dosed at long intervals (once a week, once every two or three weeks, or once a month, for example) and may have more favorable side effect profiles.

The average number of drugs per antibody in preparations of EDCs from conjugation reactions may be characterized by conventional means such as spectrophotometry, mass spectroscopy, ELISA assay, electrophoresis, and HPLC. In some instances, separation, purification, and characterization of homogeneous EDC where the number of agents is a certain value for the EDC with other drug loadings may be achieved by means such as reverse phase HPLC or electrophoresis. Liquid chromatography methods such as polymeric reverse phase (PLRP) and hydrophobic interaction (HIC) may separate EDC in a mixture by drug loading value. Preparations of EDC with a single agent loading value may be isolated. However, these EDCs with single agent loads may still be heterogeneous mixtures, because the drug moieties may be attached, via the linker, at different sites on the antibody.

### III. Treatment Methods

The EDCs of the invention are generally useful in methods of treating a patient with cancer. Such methods may comprise administering a therapeutically effective dose of a Class 1 EDC to a patient in need of treatment. In some embodiments, one or more drugs in addition to the Class 1 EDC is administered to the patient to treat the cancer including, for example, a dysadherin positive cancer. In various embodiments, the other drug is selected from the group consisting of gemcitabine, paclitaxel, TRAIL (tissue necrosis factor (TNF)-related apoptosis-inducing ligand also known as Apo2 ligand and Apo2L) (e.g., izTrail), and fibroblast growth factor (FGF) receptor kinase inhibitors (e.g., PD 173074), mTOR inhibitors (e.g., Everolimus) and glycolysis inhibitors (e.g., CEN10-128-cys). Surprisingly, the Class 1 EDCs disclosed herein have been demonstrated to synergize in promoting cell death with either TRAIL, fibroblast growth factor (FGF) receptor kinase inhibitors, mTOR inhibitors, or glycolysis inhibitors.

In a first embodiment, the patient is a lung cancer patient. In one embodiment, the lung cancer is a non-small cell lung cancer (NSCLC). In one embodiment, the lung cancer is a squamous cell carcinoma. In another embodiment, the lung cancer is a large cell carcinoma. In one embodiment, the patient is administered a second drug approved for the treatment of lung cancer in combination with the Class 1 EDC. In one embodiment, the second drug is paclitaxel or docetaxel or another taxane drug. In one embodiment, the second drug is a TRAIL, including but not limited to izTRAIL. In one embodiment, the second drug is an antibody to the receptor of TRAIL where the antibody acts like TRAIL to signal apoptosis. In one embodiment, the second drug is an FGF receptor kinase inhibitor, including but not limited to PD173074. In one embodiment, the second drug is an mTOR inhibitor, including but not limited to Everolimus. In one embodiment, the second drug is a glycolysis inhibitor, including but not limited to CEN10-128-cys. In various embodiments, the other drug is selected from the group consisting of pemetrexed, docetaxel, gefitinib, gemcitabine, vinorelbine, porfimer sodium, erlotinib, etoposide, topotecan, methotrexate, bevacizumab, carboplatin, cisplatin, and crizotinib.

In a second embodiment, the patient is a pancreatic cancer (PaCa) patient. In one embodiment, the patient is administered another drug approved for the treatment of pancreatic cancer in combination with the Class 1 EDC. In various embodiments, the other drug is gemcitabine. In other embodiments, the other drug is selected from the group consisting of fluorouracil, erlotinib, gemcitabine, sunitinib, everolimus and Mitomycin C.

In a third embodiment, the patient is a lymphoma cancer patient. In one embodiment, the lymphoma is a B-cell lymphoma. In one embodiment, the patient is administered another drug approved for the treatment of lymphoma in combination with the Class 1 EDC. In various embodiments, the other drug is selected from the group consisting of methotrexate, doxorubicin, chlorambucil, nelarabine, bendamustine, bleomycin, bortezomib, cyclophosphamide, ibritumomab tiuxetan, procarbazine, plerixafor, pralatrexate, denileukin diftitox, ofatumumab, rituximab, romidepsin, tositumomab, vinblastine, bortezomib, vinblastine, vorinostat, interferon, romidepsin, brentuximab vedotin and britumomab tiuxetan.

In these and other embodiments of the treatment methods of the invention, the therapeutically effective dose is in the range of about 0.1 mg per kg patient weight (“mg/kg”) to about 100 mg/kg. In various embodiments, the therapeutically effective dose is from about 0.1 mg/kg to about 10 mg/kg. In various embodiments, the therapeutically effective dose is from 0.25 mg/kg to 5 mg/kg. In various embodiments, the therapeutically effective dose is administered once per week or once every three weeks, and dosing is continued at that frequency until the patient is cured or the cancer progresses.

As shown in the examples below, drug loading can have a significant impact on toxicity, both to normal and cancer cells, and to half-life of a Class 1 EDC. In particular, lower drug loading, i.e., drug loading of 2 or 3 drugs per antibody, can increase serum half-life of the Class 1 EDC and decrease normal cell toxicity relative to higher drug loading, i.e., drug loading of 8 or 9 drugs per antibody. However, for some cancers, a higher drug loading provides better efficacy. Thus, in various embodiments, the number of drugs attached to each antibody (referred to as “drug loading”) of a Class 1 EDC of the invention ranges from 2 to 9. In one embodiment, the drug loading is 2. In another embodiment, the drug loading is 3. In other embodiments, the drug loading is 5, 7, or 9.

In various embodiments, the drug attached to the antibody is a steroid drug that binds to the alpha subunit of the Na,K-ATPase signaling complex. In various embodiments, the steroid is digitoxigenin or scillarenin. In various embodiments, the drug is attached to the linker via an amide bond to a glycoside, the glycosidic bond of which is formed between C1 of the appended glycoside and C3 of the steroid aglycone. The glycoside can be, for example, selected from the group consisting of 4-amino-riboside and 4-amino-xyloside, and the PEG portion of the linker is attached to the amino group of the glycoside. In various embodiments of the Class 1 EDCs of the invention, the steroid is digitoxigenin or scillarenin, and the glycoside is either 4-amino-riboside or 4-amino-xyloside.

In various embodiments, the drug is attached to the linker via the C1 hydroxyl group of a glycoside, the glycoside is 4-amino-riboside or 4-amino-xyloside, and the PEG portion of the linker is attached to the amino group of the glycoside portion of the linker. In various embodiments, the PEG portion of the linker contains from 2 to 36 glycol units. In various embodiments, the PEG portion of the linker contains 24 glycol units.

In various embodiments, the antibody is an M53 monoclonal antibody. In various embodiments, the antibody is a monoclonal antibody that binds to the same epitope as the M53 antibody. In various embodiments, the antibody is a chimeric form of the M53 antibody. In various embodiments, the antibody is a humanized form of the M53 antibody.

The administration of the compounds according to the invention can be done by any of the administration methods accepted for the therapeutic agents and generally known in the art. These processes include, but are not limited to, systemic administration, for example by parenteral, oral, nasal, or topical administration. Parenteral administration is done generally by subcutaneous, intramuscular or intravenous injection, or by perfusion. In general, antibody based therapeutics such as the EDC of the invention are typically administered intravenously. The injectable compositions can be prepared in standard forms, either in suspension or liquid solution or in solid form that is suitable for an extemporaneous dissolution in a liquid. In one embodiment, parenteral administration uses the installation of a system with slow release or extended release that ensures the maintenance of a constant dose level.

For the treatment of disease, the appropriate dosage of an EDC will depend on the type of disease to be treated, the severity and course of the disease, previous therapy, the patient's clinical history and response to the antibody, and the discretion of the attending physician. Thus, the dosage for the administration of compounds according to the invention is selected according to a variety of factors including the type, strain, age, weight, sex and medical condition of the subject; the severity of the condition to be treated; the method of administration; the condition of the renal and hepatic functions of the subject and the nature of the particular EDC administered. A normally experienced doctor will easily determine and prescribe the effective amount of the desired EDC to treat the medical condition that is to be treated. By way of examples, when given parenterally, the effective levels of the Class 1 EDC according to the invention will be in the range of from about 0.1 to about 10 mg per kg of body weight, e.g. from about 0.25 mg to about 2.5 mg per kg of body weight. The Class 1 EDC will generally be administered weekly or biweekly or every three weeks, when administered intravenously.

The EDCs of the invention may be used to treat various diseases or disorders, such as cancer and autoimmune conditions in human or animal subjects. In one embodiment, the subject is a human. In another embodiment, the subject is a non-human animal (e.g dog, cat, horse, bird, etc.) Exemplary conditions or disorders include benign or malignant tumors; leukemia and lymphoid malignancies; other disorders such as neuronal, glial, astrocytal, hypothalamic, glandular, macrophagal, epithelial, stromal, blastocoelic, inflammatory, angiogenic and immunologic disorders.

Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, gastrointestinal stromal tumor (GIST), pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In addition to cancer, the EDCs of the invention can be used as anti-inflammatory agents or to treat other diseases. Studies suggest the Na,K-ATPase subunit isoform/modulator distribution and levels in the lungs of cystic fibrosis patients are distinct from those of a normal lung, and so are a target for therapeutic agents against cystic fibrosis hyperinflammation. Studies reveal that cardiac glycosides that bind to the Na,K-ATPase can suppress hypersecretion of IL-8 from cultured CF epithelial cells via specific inhibition phosphorylation of a NF-kappa B inhibitor (see Srivastava, M., et. al. Proc. Natl. Acad. Sci. USA 2004, 101, 7693-7698, incorporated herein by reference). A review of the potential therapeutic uses of cardiac glycosides discusses obesity, kidney disease, migraines, epilepsy, dystonia, Parkinsonism (2007 Journal of Internal Medicine 261; 44-52).

The Class 1 EDC herein can be administered concurrently, sequentially, or alternating with a second drug or upon non-responsiveness with other therapy. Thus, the combined administration of a second drug includes co-administration, using separate formulations or a single pharmaceutical formulation, and consecutive administration in either order, wherein preferably there is a time period while both (or all) therapies simultaneously exert their biological activities. Multiple second drugs may be used in combination the EDC of the invention. Thus, an EDC of the invention may be combined in a pharmaceutical combination formulation, or dosing regimen as combination therapy, with a second compound having anti-cancer properties. The second compound of the pharmaceutical combination formulation or dosing regimen preferably has complementary activities to the EDC of the combination such that they do not adversely affect each other.

The second compound may be a chemotherapeutic agent, cytotoxic agent, cytokine, growth inhibitory agent, anti-hormonal agent, aromatase inhibitor, protein kinase inhibitor, lipid kinase inhibitor, anti-androgen, antisense oligonucleotide, ribozyme, gene therapy vaccine, anti-angiogenic agent and/or cardioprotectant. Such molecules are suitably present in combination in amounts that are effective for the purpose intended. A pharmaceutical composition containing an EDC may also have a therapeutically effective amount of a chemotherapeutic agent such as a tubulin-forming inhibitor, a topoisomerase inhibitor, or a DNA binder.

In one embodiment, the combination therapeutic agent is selected from a TRAIL or an agonist mAB having similar activity; Bevacizumab; Carboplatin; Cisplatin; Cyclophosphamide; Docetaxel injection; Doxorubicin; Etoposide; Etoposide Phosphate; Gemzar (gemcitabine HCL); Hycamtin (topotecan hydrochloride); Ifosfamide; Iressa (gefitinib); Irinotecan injection; Methotrexate injection; Mitomycin; Paclitaxel; Photo fin, QLT; Premetrexed; Procarbazine; Streptozocin; Tarceva (erlotinib); Vinblasine; Vincristine; and Vinorelbine tartrate.

Thus, other therapeutic regimens may be combined with the administration of a Class 1 EDC in accordance with this invention. The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein there is a time period while both (or all) active agents simultaneously exert their biological activities.

In one embodiment, treatment with an EDC of the present invention involves the combined administration of an anticancer agent identified herein, and one or more chemotherapeutic agents or growth inhibitory agents, including coadministration of cocktails of different chemotherapeutic agents. Chemotherapeutic agents include taxanes (such as paclitaxel and doxetaxel) and/or anthracycline antibiotics. Preparation and dosing schedules for such chemotherapeutic agents may be used according to manufacturers's instructions or as determined empirically by the skilled practitioner. Preparation and dosing schedules for such chemotherapy are also described in Chemotherapy Service Ed., M. C. Perry, Williams & Wilkins, Baltimore, Md. (1992).

The anticancer agent may be combined with an anti-hormonal compound; e.g., an anti-estrogen compound such as tamoxifen; ananti-progesterone such as onapristone (EP 616812); or an anti-androgen such as flutamide, in dosages known for such molecules. Where the cancer to be treated is hormone independent cancer, the patient may previously have been subjected to anti-hormonal therapy and, after the cancer becomes hormone independent, the anti-ErbB2 antibody (and optionally other agents as described herein) may be administered to the patient. It may be beneficial to also coadminister a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy.

Suitable dosages for any of the above coadministered agents are those presently used and may be lowered due to the combined action (synergy) of the newly identified agent and other chemotherapeutic agents or treatments.

The combination therapy may provide an effect achieved when the active ingredients used together is greater than the sum of the effects that results from using the compounds separately. The effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, an effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

Therapeutic EDCs of the invention may be administered by any route appropriate to the condition to be treated. The EDC will typically be administered parenterally, i.e. infusion, subcutaneous, intramuscular, intravenous, intradermal, intraperitoneal, intrathecal, bolus, intratumor injection or epidural (Shire et al (2004) J. Pharm. Sciences 93(6):1390-1402).

### IV. Pharmaceutical Formulations and Unit Dose Forms

The present invention provides pharmaceutical formulations of Class 1 EDC suitable for parenteral, including but not limited to intravenous, administration. In one embodiment, the invention provides pharmaceutical formulations suitable for parenteral administration that comprise a Class 1 EDC in combination with a pharmaceutically acceptable vehicle, vector, diluent, and/or excipient. The present invention also provides unit dose forms of these pharmaceutical formulations. In one embodiment, the invention provides a unit dose form containing a pharmaceutical formulation of the invention suitable for intravenous administration that contains from about 2.5 mg to about 1.5 g of a Class 1 EDC. In various embodiments, these unit dose forms contain 5 mg, 10 g, 25 mg, 0.5 g, or 1 g of a Class 1 EDC.

Pharmaceutical formulations of EDCs are typically prepared for parenteral administration with a pharmaceutically acceptable parenteral vehicle and in a unit dosage injectable form. An EDC having the desired degree of purity is optionally mixed with pharmaceutically acceptable diluents, carriers, excipients or stabilizers, in the form of a lyophilized formulation or an aqueous solution (Remington's Pharmaceutical Sciences (1980) 16th edition, Osol, A. Ed.).

Acceptable parenteral vehicles, diluents, carriers, excipients, and stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as Tween, PLURONICS®, or polyethylene glycol (PEG). For example, lyophilized anti-ErbB2 antibody formulations are described in WO 97/04801, expressly incorporated herein by reference. An exemplary formulation of an EDC contains about 100 mg/ml of trehalose (2-(hydroxymethyl)-6-[3,4,5-trihydroxy-6-(hydroxymethyl)tetrahydropyran-2-yl]oxy-tetrahydropyran-3,4,5-triol; C₁₂H₂₂O₁₁; CAS Number 99-20-7) and about 0.1% TWEEN™ (polysorbate 20; dodecanoic acid 2-[2-[3,4-bis(2-hydroxyethoxy)tetrahydrofuran-2-yl]-2-(2-hydroxyethoxy)et-hoxy]ethyl ester; C₂₆H₅₀O₁₀; CAS Number 9005-64-5) at approximately pH 6.

Pharmaceutical formulations of a therapeutic EDC may contain certain amounts of unreacted drug moiety (D), antibody (or other targeting moiety)-linker intermediate (Ab-L), and/or drug-linker intermediate (D-L), as a consequence of incomplete purification and separation of excess reagents, impurities, and by-products, in the process of making the EDC; or time/temperature hydrolysis or degradation upon storage of the bulk EDC or formulated EDC composition. For example, it may contain a detectable amount of drug-linker or various intermediates. Alternatively, or in addition to, it may contain a detectable amount of the un-linked free targeting moiety. An exemplary formulation may contain up to 10% molar equivalent of the agent of agent linker as it was determined by the in vitro cellular proliferation assays that in some cases the drug-linker conjugate less potent in cell killing than free drug.

The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi permeable matrices of solid hydrophobic polymers containing the EDC, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile, which is readily accomplished by filtration through sterile filtration membranes.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Aqueous suspensions contain the active materials (EDC) in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The pharmaceutical compositions of EDC may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, an aqueous solution intended for intravenous infusion may contain from about 3 to 500.mu.g of the active ingredient per milliliter of solution in order that infusion of a suitable volume at a rate of about 30 mL/hr can occur. Subcutaneous (bolus) administration may be effected with about 1.5 ml or less of total volume and a concentration of about 100 mg EDC per ml. For EDC that require frequent and chronic administration, the subcutaneous route may be employed, such as by pre-filled syringe or autoinjector device technology.

As a general proposition, the initial pharmaceutically effective amount of EDC administered per dose will be in the range of about 0.1-10 mg/kg, namely about 0.25 to 5 mg/kg of patient body weight per day, with the typical initial range of compound used being 0.25 to 5 mg/kg/day (often, once weekly dosing or even less frequent dosing will be employed). For example, human patients may be initially dosed at about 0.25 mg EDC per kg patient body weight. The dose may be escalated to the maximally tolerated dose (MTD). The dosing schedule may be about once every week or once every 3 weeks, but according to diagnosed condition or response, the schedule may be more or less frequent. The dose may be further adjusted during the course of treatment to be at or below MTD which can be safely administered for multiple cycles, such as about 4 or more.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Exemplary unit dosage formulations contain a daily dose or unit daily sub-dose, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The compositions according to the invention can be sterilized and/or can contain one or more of: non-toxic adjuvants and auxiliary substances such as agents for preservation, stabilization, wetting or emulsification; agents that promote dissolution; and salts to regulate osmotic pressure and/or buffers. In addition, they can also contain other substances that offer a therapeutic advantage. The compositions are prepared, respectively, by standard processes of mixing, granulation or coating well known to those skilled in the art.

In another embodiment of the invention, articles of manufacture containing materials useful for the treatment of the disorders described above are provided. In one aspect, the article of manufacture comprises (a) a container comprising the compounds herein (preferably the container comprises the EDC and a pharmaceutically acceptable carrier or diluent within the container); and (b) a package insert with instructions for treating the disorder in a patient.

Thus, in another embodiment, an article of manufacture, or “kit”, containing EDC and materials useful for the treatment of the disorders described above is provided. The article of manufacture comprises a container and a label or package insert on or associated with the container. Suitable containers include, for example, bottles, vials, syringes, or blister pack. The containers may be formed from a variety of materials such as glass or plastic. The container holds an EDC composition which is effective for treating the condition and may have a sterile access port (for example the container may be an intravenous solution bag or a vial having a stopper pierceable by a hypodermic injection needle). At least one active agent in the composition is an EDC. The label or package insert indicates that the composition is used for treating the condition of choice, such as cancer. For example, the cancer may be one which overexpresses one of the targets of the EDC of the invention. The label or package insert may also indicate that the composition can be used to treat cancer, wherein the cancer is not characterized by overexpression of one of the targets of the EDC of the invention. In other embodiments, the package insert may indicate that the EDC composition can be used also to treat hormone independent cancer, prostate cancer, colon cancer or colorectal cancer.

The article of manufacture may comprise a container with a compound contained therein, wherein the compound comprises an EDC of the present invention. The article of manufacture in this embodiment may further comprise a package insert indicating that the EDC can be used to treat cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

Further advantages and characteristics of the invention will emerge from the following Examples, given by way of illustration and which are not to be construed as limiting, and in which reference will be made to the accompanying drawings. It will be clear that the invention may be practiced otherwise than as particularly described in the foregoing Examples. Numerous modifications and variations of the present invention are possible in light of the above teachings and, therefore, are within the scope of the appended claims following these Examples. The Examples are divided into an Agent Synthesis section, an Antibody section and an EDC section.

### Example 1

### Synthesis of Linker-Ready Therapeutic Agents

This example describes synthetic protocols for attaching a steroid drug to a linker to produce “linker-ready” agents that can be readily attached to an antibody, as described herein. The linker-ready agents can also be used as controls in studies to investigate activity of potential EDC breakdown products, as may be generated by EDC degradation by proteases in vivo. The linker-ready reagents described in this example include PEG24-CEN09-106, PEG24-CEN09-107, PEG24-CEN10-110 and PEG24-CEN-319.

PEG24-CEN09-106 is a scillarenin based linker-ready agent that comprises a steroid, a linker and an active maleimide group. The general synthetic steps for the preparation of PEG24-CEN09-106 are as follows.

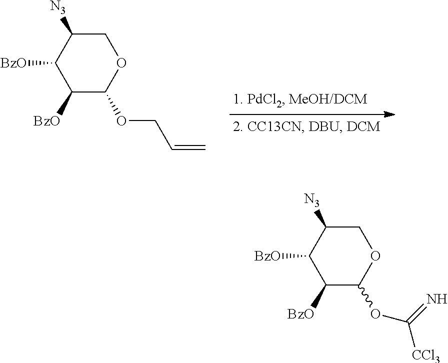

### 2,3-di-O-benzoyl-4-azido-4-deoxy-L-xylopyranoside-1-trichloroacetimidate

1-Allyl-2,3-di-O-benzoyl-4-azido-4-deoxy-L-ribopyranoside (11.9 g, 28.1 mmol) was dissolved in dichloromethane/methanol (80 mL, 90:10) under argon and PdCl₂ (0.5 g, 2.8 mmol) was added to the solution. The mixture was stirred overnight at room temperature, filtered through a pad of Celite and concentrated under reduced pressure. The residue was filtered through a pad of silica gel (hexane/EtOAc, 70:30). The resulting compound (8.38 g, 21.83 mmol) was dissolved in dry dichloromethane (170 mL) under argon. CCl₃CN (21.9 mL, 218.3 mmol) was added, followed by dropwise addition of DBU (1.63 mL, 10.91 mmol) at 0° C. The reaction was stirred for 1 h at 0° C. The solvent was removed under reduced pressure. The crude product was filtered through a pad of silica gel (hexane/EtOAc, 60:40 to 40:60) to afford 2,3-di-O-benzoyl-4-azido-4-deoxy-L-ribopyranosid-1-trichloroacetimidate as a yellow oil (9.7 g, 65%). The compound was carried forward without further purification. R_{f} 0.37 (silica gel, hexane/EtOAc, 80:20).

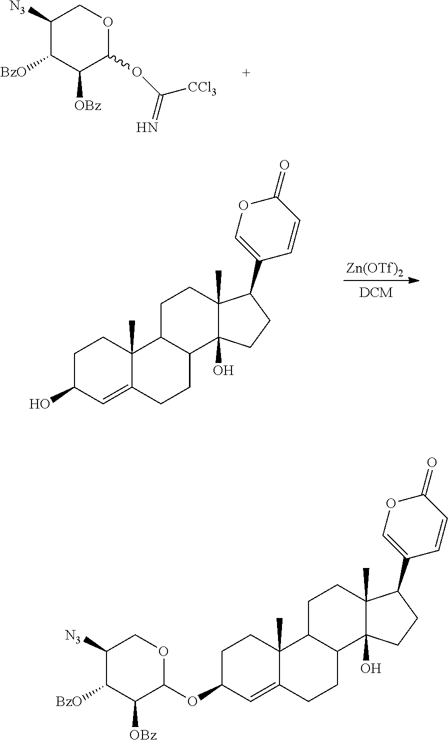

### Scillarenin-2,3-di-O-benzoyl-4-azido-4-deoxy-L-xylopyranoside

2,3-di-O-benzoyl-4-azido-4-deoxy-L-xylopyranoside-1-trichloroacetimidate (0.483 g, 0.915 mmol) was added to a suspension of activated 4 Å molecular sieves (90 mg) in dry dichloromethane (15 mL) under argon at 0° C. Scillarenin (0.182 g, 0.474 mmol) was then added to the mixture. After 5 minutes, Zn(OTf)₂ (17 mg, 0.047 mmol) was added and the reaction mixture was stirred for an additional 30 minutes at 0° C. An additional amount of scillarenin (0.182 g, 0.474 mmol) was added. The reaction mixture was stirred for 30 minutes at 0° C. The reaction was quenched with few drops of Et₃N. The mixture was filtered and the solvent was removed under reduced pressure. The crude product was purified by flash chromatography (hexane/EtOAc, 75:25 to 50:50) to afford scillarenin-2,3-di-O-benzoyl-4-azido-4-deoxy-L-xylopyranoside as a white powder (0.521 g, 76%) R_{f} 0.35 (silica gel, hexane/EtOAc, 50:50). ¹H-NMR (300 MHz, CDCl₃), 0.68 (s, 3H), 0.90-2.17 (m, 21H), 2.39-2.44 (m, 1H), 3.47 (dd, 1H, J=12.0, 9.5 Hz, H-5b), 3.79-3.87 (m, 1H, H-4), 4.17-4.22 (m, 2H, H-5a), 4.78 (d, 1H, J=6.8 Hz, H-1), 5.26 (dd, 1H, J=8.6, 6.8 Hz, H-2), 5.33 (s, 1H), 5.49 (dd, 1H, J=8.7 Hz, H-3), 6.22 (dd, 1H, J=9.7, 0.6 Hz), 7.18-7.19 (m, 1H), 7.33-7.39 (m, 4H), 7.47-7.53 (m, 2H), 7.80 (dd, 1H, J=9.7, 2.6 Hz), 7.92-7.97 (m, 4H); ¹³C-NMR (75 MHz, CDCl₃) 16.7, 19.0, 21.4, 25.8, 28.7, 28.8, 32.4, 32.8, 35.2, 37.6, 40.8, 42.9, 48.4, 50.2, 51.2, 59.2, 63.1, 71.6, 72.9, 76.1, 85.2, 100.0, 115.5, 121.7, 122.8, 128.5, 128.6, 129.1, 129.5, 129.9, 130.1, 133.4, 133.6, 146.9, 147.6, 148.7, 162.5, 165.3, 165.7.

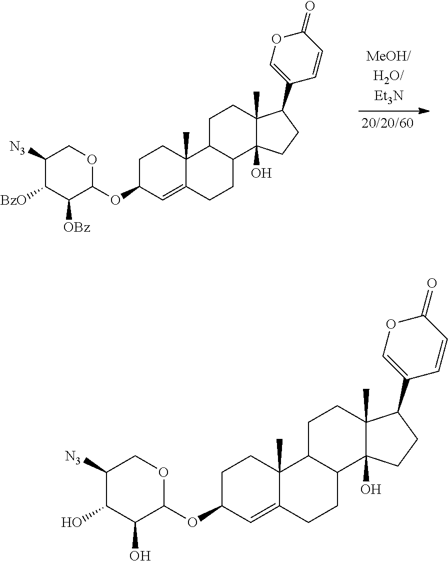

### Scillarenin-4-azido-4-deoxy-L-xylopyranoside

Scillarenin-2,3-di-O-benzoyl-4-azido-4-deoxy-L-xylopyranoside (0.351 g, 0.468 mmol) was dissolved in methanol (21 mL). Et₃N (7 mL) and H₂O (7 mL) were added. The reaction mixture was stirred for 2 days at room temperature. The mixture was filtered and the solvent was stripped under reduced pressure. The crude product was purified by flash chromatography (CH₂Cl₂/MeOH, 98:2 to 95:5) to afford scillarenin-4-azido-4-deoxy-L-xylopyranoside as a yellow powder (40 mg, 24%) R_{f} 0.31 (CH₂Cl₂/MeOH, 95:5); ¹H-NMR (300 MHz, CD₃OD), 0.74 (s, 3H), 1.03-2.21 (m, 21H), 2.52-2.57 (m, 1H), 3.12-3.20 (m, 2H), 3.40-3.44 (m, 2H), 3.87-3.92 (m, 1H), 4.17-4.23 (m, 1H), 4.31 (d, 1H, J=7.7 Hz, H-1), 5.35 (s, 1H), 6.28 (dd, 1H, J=9.7, 0.8 Hz), 7.43 (d, 1H, J=1.5 Hz), 7.99 (dd, 1H, J=9.7, 2.6 Hz).

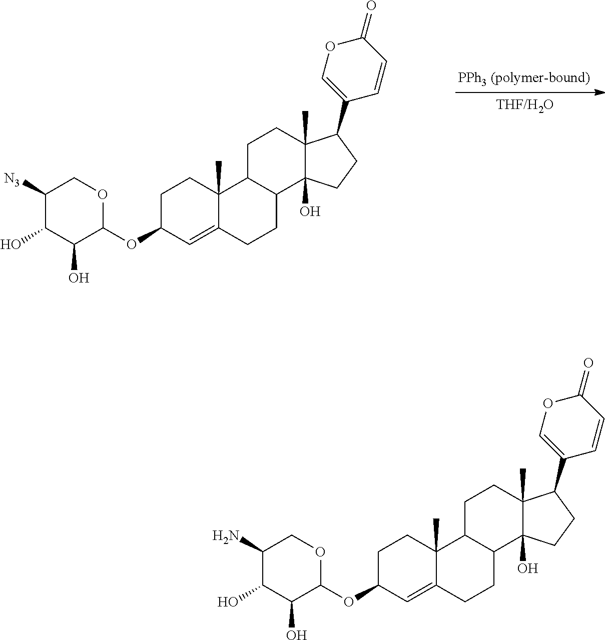 [SMILES: C[C@]12CC[C@H](OC3OC[C@H](N=[N+]=[N-])[C@@H](O)[C@@H]3O)C=C1CCC1C2CC[C@]2(C)[C@@H](c3ccc(=O)oc3)CC[C@]12O ; C[C@]12CC[C@H](OC3OC[C@H](N)[C@@H](O)[C@@H]3O)C=C1CCC1C2CC[C@]2(C)[C@@H](c3ccc(=O)oc3)CC[C@]12O]

### Scillarenin-4-amino-4-deoxy-L-xylopyranoside

Scillarenin-4-azido-4-deoxy-L-xylopyranoside (1.61 g, 2.34 mmol) was dissolved in THF/H₂O (2.8 mL, 90:10). PPh₃ polymer-bound (79 mg, 3 mmol.g⁻¹) was added. The reaction mixture was stirred for 2 hours at 40° C. The mixture was then filtered and the solvent was removed under reduced pressure. The crude product was purified by flash chromatography (CH₂Cl₂/MeOH, 90:10 to 80:20) to afford scillarenin-4-amino-4-deoxy-L-xylopyranoside as a yellow powder (23 mg, 58%) R_{f} 0.2 (CH₂Cl₂/MeOH, 80:20); ¹H-NMR (300 MHz, CD₃OD), 0.74 (s, 3H), 1.06-2.19 (m, 21H), 2.52-2.57 (m, 1H), 2.75-2.86 (m, 1H, H-4), 3.14-3.24 (m, 2H, H-2, H-3), 3.64-3.72 (m, 1H, H-5b), 3.87-3.91 (m, 1H, H-5a), 4.19-4.24 (m, 1H), 4.36 (d, 1H, J=7.1 Hz, H-1), 5.38 (s, 1H), 6.28 (dd, 1H, J=9.7, 0.6 Hz), 7.42 (d, 1H, J=1.6 Hz), 7.99 (dd, 1H, J=9.7, 2.5 Hz); ¹³C-NMR (75 MHz, CD₃OD) 17.4, 19.6, 22.5, 26.8, 29.9, 30.1, 33.3, 33.6, 36.6, 38.8, 41.8, 43.5, 49.4, 51.7, 52.2, 75.3, 76.5, 78.9, 79.3, 79.8, 85.8, 103.7, 115.6, 123.4, 125.1, 148.4, 149.4, 150.5, 164.9.

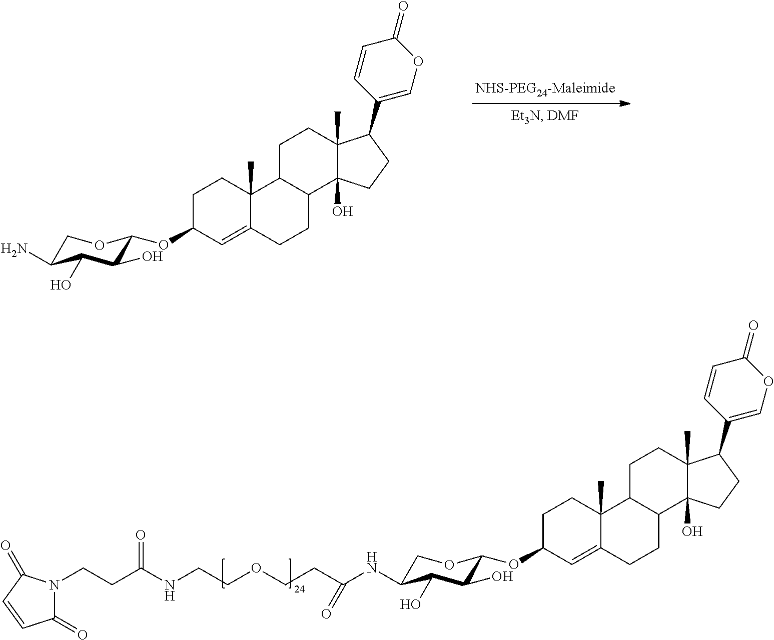

PEG24-CEN09-106.

To a solution of Scillarenin-4-amino-4-deoxy-L-xylopyranoside (18.5 mg, 0.0359 mmol) in DMF (1 mL) at room temperature was added NHS-PEG₂₄-Maleimide (50 mg, 0.0359 mmol). Then Et₃N (0.025 mL, 0.18 mmol) was added. The reaction was stirred at room temperature for 2 hours. The solvent was removed under reduced pressure. The crude material was purified by flash chromatography (CH₂Cl₂/MeOH, 95:5 to 80:20) to afford PEG24-CEN09-106 as a yellow oil (48 mg, 75%) R_{f} 0.66 (CH₂Cl₂/MeOH, 80:20). HPLC analysis [Luna C18, 250×4.60 mm, 5 μm, 5% to 95% ACN over 32 minutes, 1 ml.min⁻¹] indicated a product which was >95% pure. HRMS-ESI (m/z): calcd for C₈₇H₁₄₇N₃O₃₅ [M+K⁺]⁺: 1832.9452, found 1832.9777. The NHS-(PEG)ₙ-maleimide (where n is an integer) can be attached to any amine-bearing molecule (vide infra) using similar reaction conditions.

PEG24-CEN09-107 is a scillarenin based linker-ready agent that comprises a steroid, a linker and an active maleimide group. It differs from PEG24-CEN09-106 in that the linker contains a 4-amino-riboside instead of a 4-amino-xyloside sugar. The general synthetic steps for the preparation of PEG24-CEN09-107 are as follows.

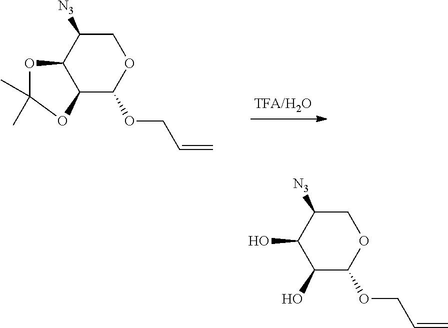 [SMILES: C=CCO[C@H]1OC[C@H](N=[N+]=[N-])[C@@H]2OC(C)(C)O[C@H]12 ; C=CCO[C@H]1OC[C@H](N=[N+]=[N-])[C@H](O)[C@@H]1O]

### 1-Allyl-4-azido-4-deoxy-L-ribopyranoside

1-Allyl-2,3-O-isopropylidene-4-azido-4-deoxy-L-ribopyranoside (8.53 g, 33.6 mmol) was dissolved in TFA/H₂O (80:20, 40 mL). The reaction mixture was stirred for 30 min at 0° C. The solvents was removed under reduced pressure and the resulting residue was purified by flash chromatography (CH₂Cl₂/MeOH, 95:5) to give 1-allyl-4-azido-4-deoxy-L-ribopyranoside as brown oil (26.3 g, 72%) R_{f} 0.5 (CH₂Cl₂/MeOH, 95:5); ¹H-NMR (300 MHz, CD₃OD), 3.49 (dd, 1H, J=5.1, 3.5 Hz, H-2), 3.58 (ddd, 1H, J=6.7, 3.9, 3.2 Hz, H-4), 3.75 (dd, 1H, J=11.6, 6.7, H-5b), 3.83 (dd, 1H, J=11.6, 3.9 Hz, H-5a), 4.05 (ddt, 2H, J=1.4, 6.0, 13.0 Hz, CH₂—CH═CH₂), 4.09 (dd, 1H, J=3.2 Hz, H-3), 4.23 (ddt, 1H, J=1.5, 5.2, 13.0 Hz, CH₂—CH═CH₂), 4.70 (d, 1H, J=5.1 Hz, H-1), 5.17 (ddd, 1H, J=1.4, 2.9, 10.4 Hz, CH₂═CH), 5.30 (ddd, 1H, J=1.7, 3.4, 17.3 Hz, CH₂═CH), 6.00-5.87 (m, 1H, CH₂═CH).

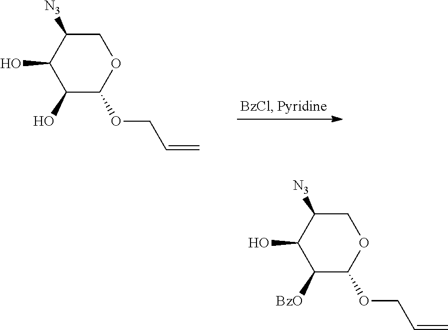

### 1-Allyl-2-O-benzoyl-4-azido-4-deoxy-L-ribopyranoside

1-Allyl-4-azido-4-deoxy-L-ribopyranoside (4.0 g, 18.6 mmol) was dissolved in dry dichloromethane (120 mL) under argon. Pyridine (4.5 mL, 55.76 mmol) was added and the mixture was stirred for 30 min at −30° C. BzCl (2.25 mL, 19.51 mmol) was then added drop wise. It was then stirred overnight at room temperature. The solvent was removed under reduced pressure. The resulting residue was dissolved in EtOAc, washed with water, 0.1N HCl and brine. The organic layer was dried over Na₂SO₄, filtered and concentrated. The crude product was purified by flash chromatography (Toluene/EtOAc, 95:5 to 90:10) to give 1-allyl-2-O-benzoyl-4-azido-4-deoxy-L-ribopyranoside as brown oil (4.46 g, 75%) R_{f} 0.45 (Toluene/EtOAc, 90:10). ¹H-NMR (300 MHz, CD₃OD), 3.77-3.86 (m, 2H, H-4, H-5b), 3.96 (dd, 1H, J=2.8, 11.7 Hz, H-5a), 4.02-4.10 (m, 1H, CH₂—CH═CH₂), 4.20-4.27 (m, 1H, CH₂—CH═CH₂), 4.36 (dd, 1H, J=3.4 Hz, H-3), 4.95 (d, 1H, J=3.9 Hz, H-1), 5.15-5.33 (m, 2H, CH₂═CH), 5.85-5.98 (m, 1H, CH₂═CH), 7.47-7.53 (m, 2H, H—Ar), 7.59-7.65 (m, 1H, H—Ar), 8.10-8.17 (m, 2H, H—Ar); ¹³C-NMR (75 MHz, CD₃OD) 60.1 (C-4), 62.3 (C-5), 67.7 (C-3), 70.1 (CH₂—CH═CH₂), 73.1 (C-2), 98.7 (C-1), 117.7 (CH₂═CH), 129.65 (C—Ar), 131.1 (C—Ar), 134.6 (CH₂═CH), 135.6 (C—Ar), 167.7 (C═O).

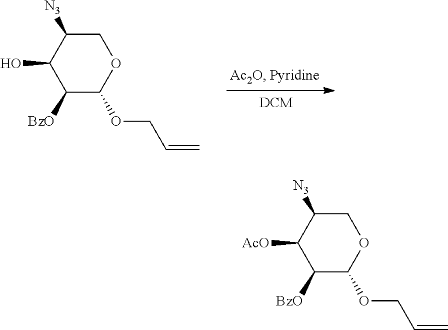

### 1-Allyl-3-O-acetyl-2-O-benzoyl-4-azido-4-deoxy-L-ribopyranoside

1-Allyl-2-O-benzoyl-4-azido-4-deoxy-L-ribopyranoside (4.45 g, 13.93 mmol) was dissolved in anhydrous pyridine (5.6 mL, 69.65 mmol) at 0° C., under argon and Ac₂O was added drop wise. The mixture was stirred overnight at room temperature. Dichloromethane was then added; the organic layer was washed with water, 0.1N HCl and brine, dried over Na₂SO₄, filtered and concentrated. The crude product was purified by flash chromatography (Toluene/EtOAc, 90:10) to give 1-allyl-3-O-acetyl-2-O-benzoyl-4-azido-4-deoxy-L-ribopyranoside as a yellow oil (5.03 g, 100%) R_{f} 0.50 (Toluene/EtOAc, 90:10); ¹H-NMR (300 MHz, CDCl₃) 2.01 (s, 3H), 3.81 (dd, 1H, J=12.4, 3.1 Hz, H-5b), 3.92-3.95 (m, 1H, H-4), 4.00-4.06 (m, 2H, H-5a, CH₂—CH═CH₂), 4.21 (ddt, 1H, J=12.8, 5.3, 1.4 Hz, CH₂—CH═CH₂) 4.96 (d, 1H, J=2.6 Hz, H-1), 5.18-5.34 (m, 2H, H-2, CH₂—CH═CH₂), 5.51 (dd, 1H, J=3.77 Hz, H-3), 5.82-5.95 (m, 1H, CH═CH₂), 7.42-7.48 (m, 2H), 7.55-7.60 (m, 1H), 8.12-8.15 (m, 2H); ¹³C-NMR (75 MHz, CDCl₃) 20.8 (CH₃) δ6.8 (C-4), 61.2 (C-5), 68.6 (C-3), 68.7 (C-2), 69.0 (CH₂═CH—), 97.5 (C-1), 118.3 (CH₂—CH═CH₂), 128.7 (C—Ar), 129.7 (C—Ar), 130.2 (C—Ar), 133.4 (C—Ar), 133.6 (CH₂═CH), 166.0 (C═O), 169.9 (C═O).

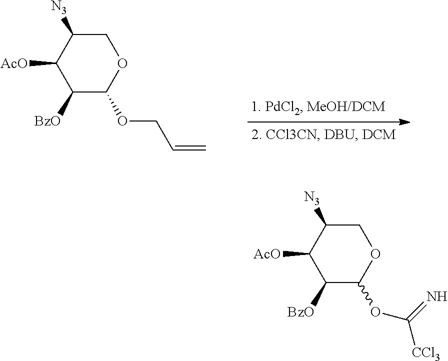

### 1-Trichloroacetimido-3-O-Acetyl-2-O-benzoyl-4-azido-4-deoxy-L-ribopyranoside

1-Allyl-3-O-acetyl-2-O-benzoyl-4-azido-4-deoxy-L-ribopyranoside (5.03 g, 13.93 mmol) was dissolved in dichloromethane/methanol (40 mL, 90:10) under argon and PdCl₂ (0.5 g, 2.6 mmol) was added. The reaction mixture was stirred overnight at room temperature. The mixture was filtered through a pad of Celite and concentrated under reduced pressure. The residue was filtered through a pad of silica gel (hexane/EtOAc, 80:20 to 50:50). The resulting compound (2 g, 6.22 mmol) was dissolved in dry DCM (50 mL) under argon and the solution was cooled to 0° C. CCl₃CN (6.24 mL, 62.2 mmol) was added, followed by dropwise addition of DBU (0.46 mL, 3.11 mmol). The reaction was stirred for 2 hours at 0° C. The solvent was removed under reduced pressure. The crude product was taken up in hexanes-EtOAc (60:40) and filtered through a pad of silica gel (hexane/EtOAc, 60:40 to 40:60) to afford 1-trichloroacetimido-3-O-Acetyl-2-O-benzoyl-4-azido-4-deoxy-L-ribopyranoside as a white gum (1.7 g, 26%) This material was carried forward without further purification. R_{f} 0.55 (hexane/EtOAc, 50:50).

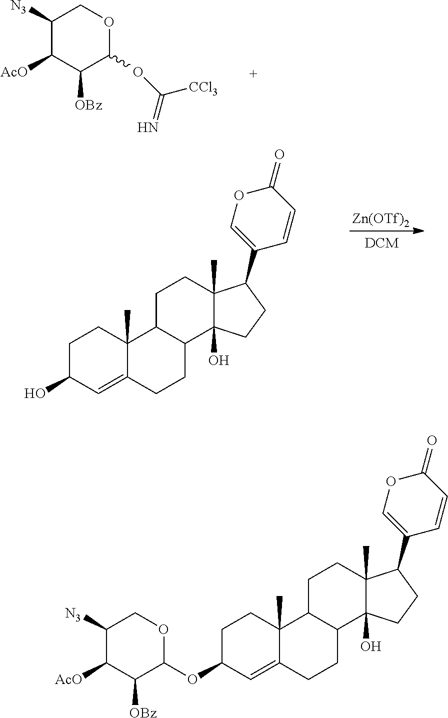

### Scillarenin-3-O-Acetyl-2-O-benzoyl-4-azido-4-deoxy-L-ribopyranoside

To a suspension of activated 4 Å molecular sieves (160 mg) in dry dichloromethane (60 mL0 at 0° C. under argon was added a solution of 1-trichloroacetimido-3-O-acetyl-2-O-benzoyl-4-azido-4-deoxy-L-ribopyranoside (5.03 g, 13.93 mmol) in the minimum amount of dry dichloromethane. Scillarenin (0.7 g, 1.825 mmol) was added and after 5 minutes at 0° C., Zn(OTf)₂ (0.133 g, 0.365 mmol) was added. The reaction mixture was stirred for 30 minutes at 0° C. Another 0.5 eq of scillarenin (0.7 g, 1.825 mmol) was added. The reaction mixture was stirred for an additional period of 30 minutes at 0° C. The reaction was quenched with few drops of Et₃N. The mixture was filtered and the solvent was removed under reduced pressure. The crude product was purified by flash chromatography (hexane/EtOAc, 60:40 to 40:60) to afford scillarenin-3-O-Acetyl-2-O-benzoyl-4-azido-4-deoxy-L-ribopyranoside as a white solid (1.64 g, 65%) R_{f} 0.39 (hexane/EtOAc, 50:50). ¹H-NMR (300 MHz, CDCl₃) 0.72 (s, 3H), 1.03-2.20 (m, 24H), 2.42-2.48 (m, 1H), 3.82 (dd, 1H, J=12.4, 3.1 Hz, H-5b), 3.94-3.97 (m, 1H), 4.12 (dd, 1H, J=12.4, 2.4 Hz, H-5a), 4.17-4.22 (m, 1H), 5.10 (d, 1H, J=2.6 Hz, H-1), 5.26 (dd, 1H, J=2.8 Hz, H-2), 5.31 (s, 1H), 5.53 (dd, 1H, J=3.75 Hz, H-3), 6.24-6.27 (m, 1H), 7.22 (d, 1H, J=1.6 Hz), 7.44-7.49 (m, 2H), 7.56-7.61 (m, 1H), 7.82 (dd, 1H, J=9.7, 2.6 Hz), 8.13-8.16 (m, 2H); ¹³C-NMR (75 MHz, CDCl₃) 16.4, 18.9, 20.6, 21.2, 25.2, 28.5, 28.6, 32.2, 32.6, 35.0, 37.5, 40.6, 42.7, 48.2, 50.1, 51.0, 56.7 (C-4), 60.9 (C-5), 67.7 (C-3), 69.1 (C-2), 73.6, 85.0, 96.2 (C-1), 115.3, 121.2, 121.6, 128.5, 129.5, 129.9, 133.3, 146.7, 147.5, 148.5, 162.3 (C═O), 165.9 (C═O), 169.7 (C═O).

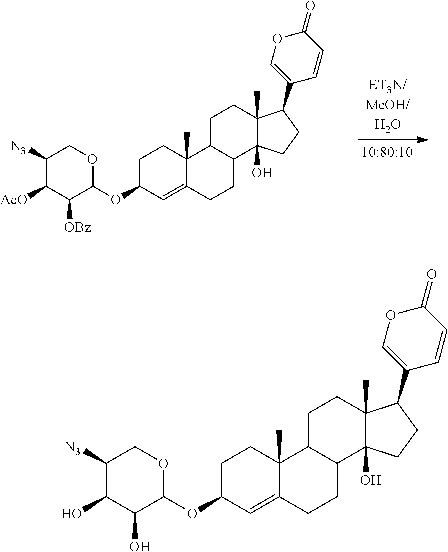

### Scillarenin-4-azido-4-deoxy-L-ribopyranoside

Scillarenin-3-O-acetyl-2-O-benzoyl-4-azido-4-deoxy-L-ribopyranoside (1.61 g, 2.34 mmol) was dissolved in methanol (20 mL. Et₃N (2.5 mL) and H₂O (2.5 mL) were added. The reaction mixture was stirred overnight at room temperature. The solvent was removed under reduced pressure. The crude was purified by flash chromatography (CH₂Cl₂/MeOH, 90:10) to afford scillarenin-4-azido-4-deoxy-L-ribopyranoside as a white solid (0.93 g, 73%) R_{f} 0.25 (CH₂Cl₂/MeOH, 90:10); ¹H-NMR (300 MHz, CD₃OD) 0.74 (s, 3H), 1.06-2.22 (m, 21H), 2.52-2.57 (m, 1H), 3.42 (dd, 1H, J=5.4, 3.2 Hz, H-2), 3.54-3.59 (m, 1H, H-4), 3.72-3.89 (m, 2H, H-5a, H-5b), 4.09-4.12 (m, 1H, H-3), 4.16-4.21 (m, 1H), 4.81 (d, 1H, J=5.4 Hz, H-1), 5.34 (s, 1H), 6.28 (dd, 1H, J=9.7, 0.7 Hz), 7.42-7.43 (m, 1H), 7.99 (dd, 1H, J=9.7, 2.6 Hz); ¹³C-NMR (75 MHz, CD₃OD) 17.3, 19.5, 22.4, 26.5, 29.8, 30.0, 33.2, 33.5, 36.5, 38.7, 41.7, 43.4, 49.6, 51.6, 52.1, 60.5 (C-4), 62.1 (C-5), 69.6 (C-3), 72.0 (C-2), 75.5, 85.7, 100.4 (C-1), 115.4, 123.1, 125.0, 148.4, 149.3, 150.5, 164.8 (C═O).

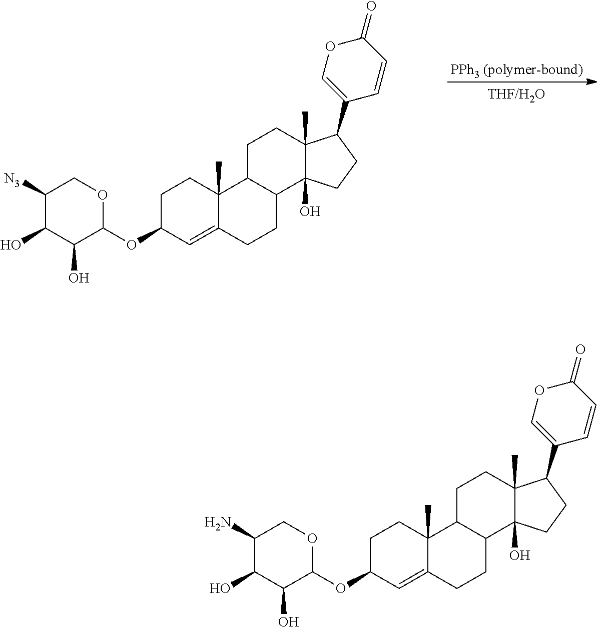 [SMILES: C[C@]12CC[C@H](OC3OC[C@H](N=[N+]=[N-])[C@H](O)[C@@H]3O)C=C1CCC1C2CC[C@]2(C)[C@@H](c3ccc(=O)oc3)CC[C@]12O ; "PPh3 (polymer-bound)" ; C[C@]12CC[C@H](OC3OC[C@H](N)[C@H](O)[C@@H]3O)C=C1CCC1C2CC[C@]2(C)[C@@H](c3ccc(=O)oc3)CC[C@]12O]

### Scillarenin-4-amino-4-deoxy-L-ribopyranoside

Scillarenin-4-azido-4-deoxy-L-ribopyranoside (1.61 g, 2.34 mmol) was dissolved in THF/H₂O (30 mL, 90:10). PPh₃ polymer-bound (2.34 g, 3 mmol.g⁻¹) was added. The mixture was stirred for 6 hours at 40° C. The mixture was filtered and the solvent was removed under reduced pressure. The crude was purified by flash chromatography (CH₂Cl₂/MeOH, 90:10 to 80:20) to afford scillarenin-4-amino-4-deoxy-L-ribopyranoside as a yellow powder (0.67 g, 73%) R_{f} 0.1 (CH₂Cl₂/MeOHl, 80:20); ¹H-NMR (300 MHz, DMSO-d₆)) 0.63 (s, 3H), 0.96-2.10 (m, 21H), 2.43-2.46 (m, 1H), 2.90-2.92 (m, 1H, H-4), 3.29 (dd, 1H, J=3.3 Hz, H-2), 3.45 (dd, 1H, J=11.4, 5.4 Hz, H-5b), 3.63-3.68 (m, 2H, H-3, H-5a), 4.02-4.08 (m, 1H), 4.69 (d, 1H, J=4.2 Hz, H-1), 5.24 (s, 1H), 6.29 (dd, 1H, J=9.7, 0.7 Hz), 7.48-7.58 (m, 1H), 7.92 (dd, 1H, J=9.7, 2.5 Hz); ¹³C-NMR (75 MHz, (CD₃)₂SO) 16.6, 18.6, 20.9, 25.2, 28.4, 28.5, 31.8, 31.9, 34.8, 37.0, 39.7, 41.5, 47.8, 49.6, 49.9, 50.8 (C-4), 63.7 (C-5), 67.1, 71.2 (C-3), 72.3 (C-3), 83.1, 99.1 (C-1), 114.2, 122.2, 122.6, 146.1, 147.3, 149.2, 161.3 (C═O).

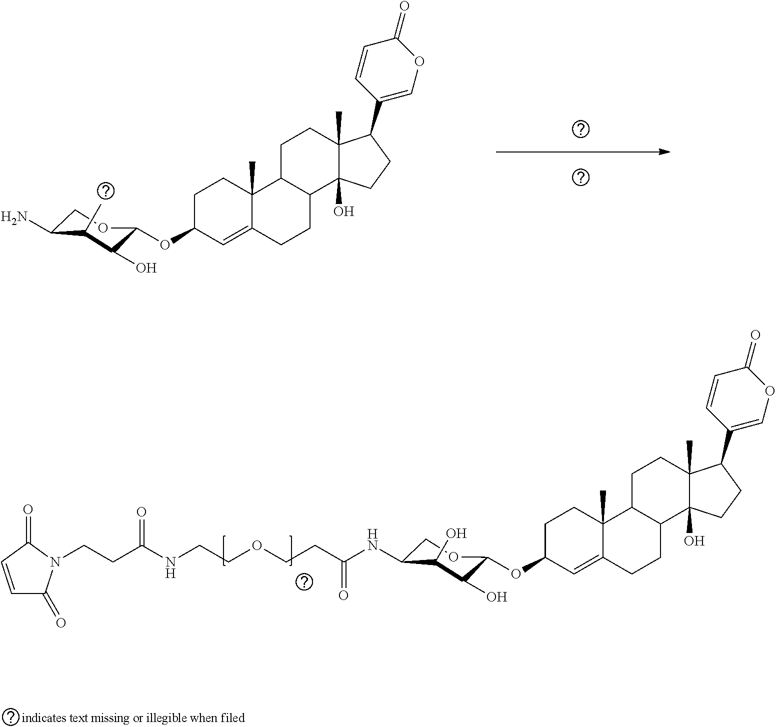

PEG24-CEN09-107.

To a solution of scillarenin-4-amino-4-deoxy-L-ribopyranoside (20 mg, 0.037 mmol) and maleimide-PEG₂₄-NHS ester (52 mg, 0.037 mmol) in N,N-dimethylacetamide (1.5 mL) was added Et₃N (0.026 mL, 0.186 mmol). The reaction mixture was stirred at RT for 1 hour. Solvent was removed in vacuo. The crude material was purified by flash chromatography (silica gel, CH₂Cl₂-MeOH 95:5 to 80:20) to afford CEN10-129 as a colorless oil (30 mg, 73%). Rₜ 15.65 min (Gemini C18, 5 μm, 4.6 mm×250 mm, 10% to 90% over 18 min, ACN, 0.1% TFA, 1 mL.min⁻¹).

PEG24-CEN10-110 is a scillarenin based linker-ready agent that comprises a steroid, a linker and an active maleimide group. It differs from PEG24-CEN09-106 in that the linker is approximately 15 angstoms longer and contains a free amine which is expected to be positively charged under physiological pH. The general synthetic steps for the preparation of PEG24-CEN10-110 are as follows.

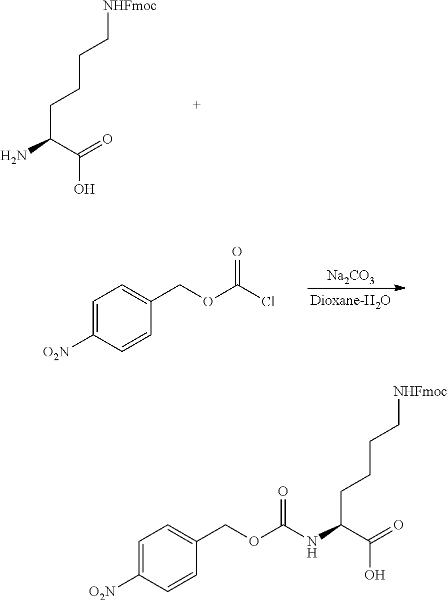

### pNZ-Lys(Fmoc)-OH

To a vigorously stirred solution of Na₂CO₃ (180 mg, 1.70 mmol) in H₂O (6 mL) was added a solution of H-Lys(Fmoc)-OH (250 mg, 0.68 mmol) in dioxane (3 mL). A solution of p-nitrobenzyloxycarbonyl chloride (161 mg, 0.74 mmol) in dioxane (3 mL) was added slowly at 0° C. The reaction mixture was stirred at 0° C. for 2.5 h, then diluted with EtOAc (20 mL) and washed with 1N HCl (20 mL), H₂O (20 mL), brine (10 mL), dried (Na₂SO₄), and concentrated. The crude material was purified by flash chromatography (silica gel, CH₂Cl₂-MeOH 98:2 to 90:10) to afford pNZ-Lys(Fmoc)-OH as a white solid (300 mg, 82%), R_{f} 0.21 (CH₂Cl₂-MeOH 90:10).

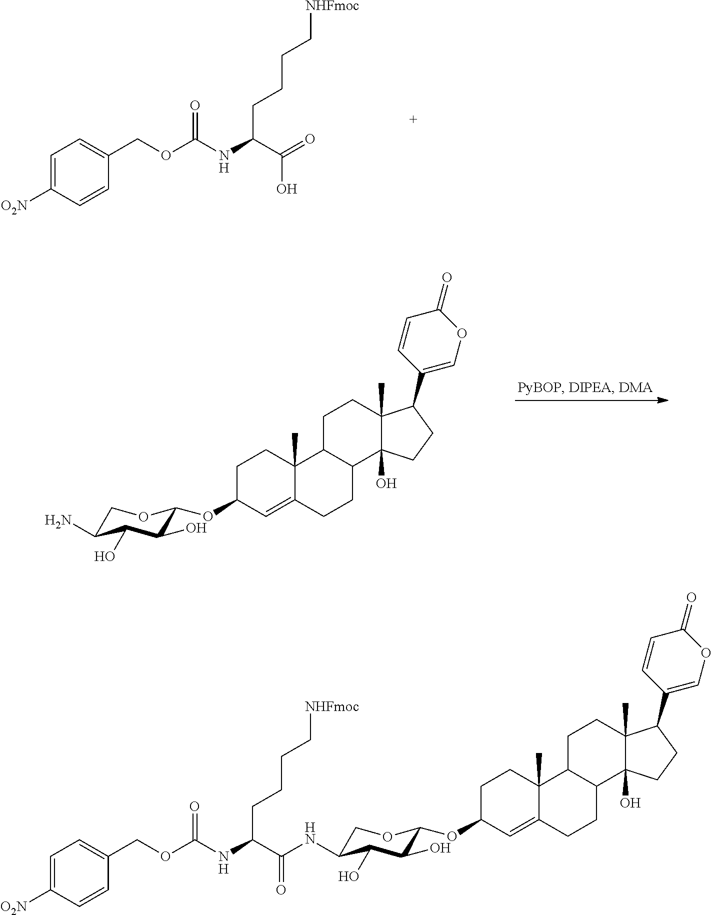

### Scillarenin-4-N-[pNZ-Lys(Fmoc)-yl]-4-deoxy-4-amino-L-xylopyranoside

Scillarenin-4-amino-4-deoxy-L-xylopyranoside (150 mg, 0.291 mmol), pNZ-Lys(Fmoc)-OH (160 mg, 0.291 mmol) and PyBOP (182 mg, 0.349 mmol) were dissolved in DMA (3 mL). Diisopropylethylamine (0.2 mL, 1.164 mmol) was added and the mixture was stirred at room temperature for 1 h. DMA was removed in vacuo. The residue was dissolved in EtOAc (20 mL), then washed with H₂O (2×10 mL), brine (10 mL), dried (Na₂SO₄), and concentrated. The crude material was purified by flash chromatography (silica gel, CH₂Cl₂-MeOH 98:2 to 90:10) to afford Scillarenin-4-N-[pNZ-Lys(Fmoc)-yl]-4-deoxy-4-amino-L-xylopyranoside as an off-white solid (288 mg, 95%), R_{f} 0.21 (CH₂Cl₂-MeOH 95:5), rt 21.82 min (Gemini C18, 5 μm, 4.6 mm×250 mm, 10% to 95% ACN, 0.1% TFA).

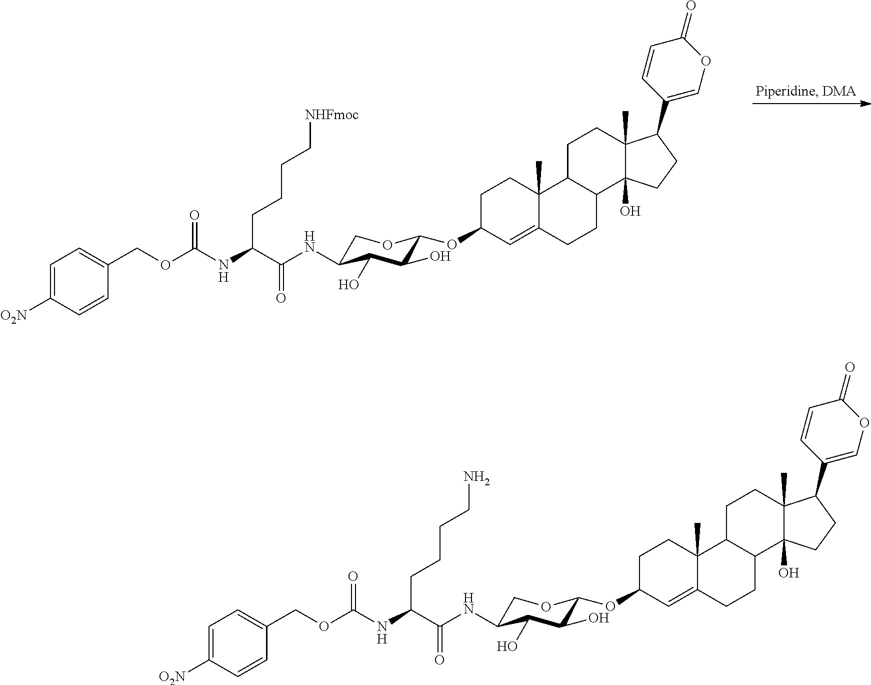

### Scillarenin-4-N-(pNZ-Lysyl)-4-deoxy-4-amino-L-xylopyranoside

Scillarenin-4-N-[pNZ-Lysyl-(Fmoc)]-4-deoxy-4-amino-L-xylopyranoside (100 mg, 0.096 mmol), was dissolved in DMA (1 mL). Piperidine (94 μL, 0.957 mmol) was added and the mixture was stirred at room temperature for 10 min. The reaction mixture was added dropwise to cold Et₂O (50 mL). The precipitate was recovered by centrifugation and washed twice with a small amount of cold Et₂O to afford Scillarenin-4-N-(pNZ-Lysyl)-4-deoxy-4-amino-L-xylopyranoside as an off-white solid (61 mg, 77%), rt 14.75 min (Gemini C18, 5 μm, 4.6 mm×250 mm, 10% to 95% ACN, 0.1% TFA).

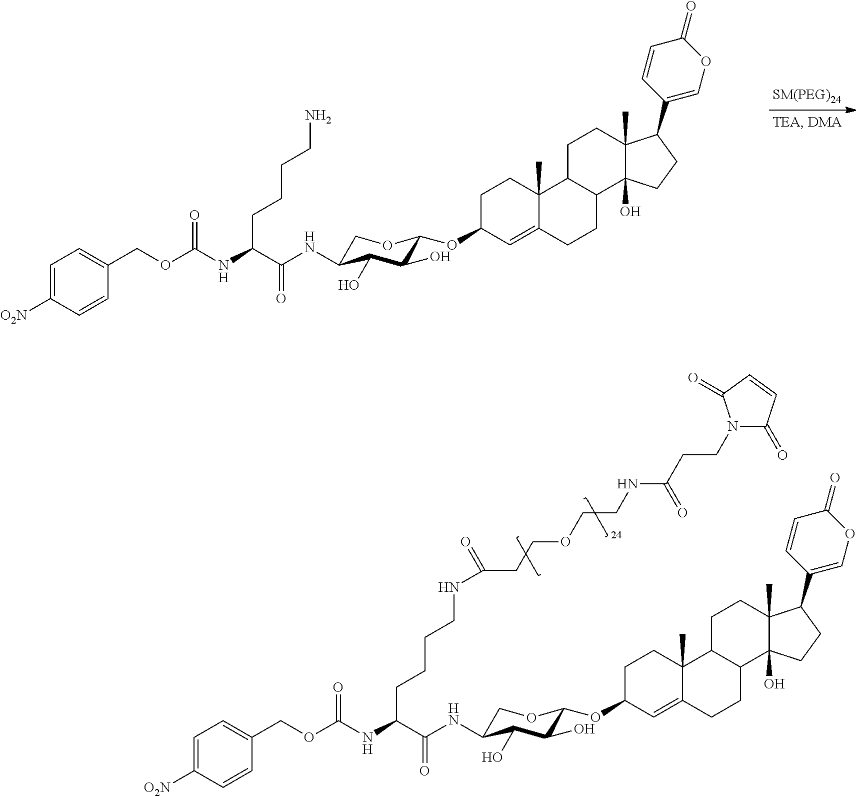

### Scillarenin-4-N-[pNZ-Lys-(Maleimide-PEG₂₄)-yl]-4-deoxy-4-amino-L-xylopyranoside

Scillarenin-[pNZ-Lys-4-amido]-4-deoxy-L-xylopyranoside (61 mg, 0.074 mmol) and Maleimide-PEG₂₄-NHS (103 mg, 0.074 mmol) were dissolved in DMA (1.5 mL). TEA (51 μL, 0.37 mmol) was added and the reaction mixture was stirred at room temperature for 20 min. The solvent was removed in vacuo. The crude material was purified by flash chromatography (silica gel, CHCl₃-MeOH—H₂O 85:15:1) to afford Scillarenin-4-N—[pNZ-Lys-(Maleimide-PEG₂₄)-yl]-4-deoxy-4-amino-L-xylopyranoside as a colorless oil (86 mg, 55%), R_{f} 0.53 (CHCl₃-MeOH—H₂O 85:15:1), rt 16.33 min (Gemini C18, 5 μm, 4.6 mm×250 mm, 10% to 95% ACN, 0.1% TFA). Maldi (m/z): calcd for C₁₀₁H₁₆₄N₆O₄₀ [M+Na]⁺: 2124.1, found 2124.1, [M+K]⁺: 2140.1, found 2140.1.

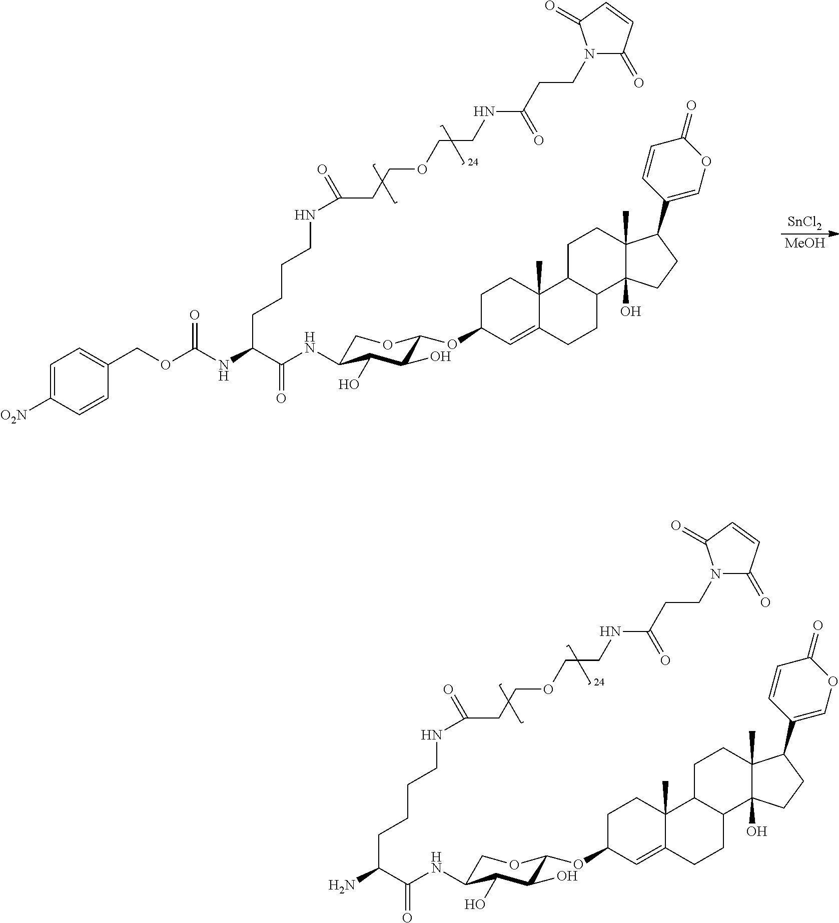

PEG24-CEN10-110.

Scillarenin-4-N-[pNZ-Lys-(Maleimide-PEG₂₄)-yl]-4-deoxy-4-amino-L-xylopyranoside (55 mg, 0.026 mmol) was dissolved in dry MeOH (2 mL). SnCl₂ (49 mg, 0.26 mmol) and 3 drops of 1.6 mM HCl were added. The reaction mixture was stirred at 45° C. for 4 h. The solvent was removed in vacuo. The crude material was purified by HPLC (rt 13.53 min, Gemini C18, 5 μm, 4.6 mm×250 mm, 10% to 95% ACN, 0.1% TFA) to afford CEN-301 (7.6 mg, 13%). Maldi (m/z): calcd for C₉₃H₁₅₉N₅O₃₆ [M+H]⁺: 1923.1, found 1923.1, [M+Na]⁺: 1945.1, found 1945.0.

PEG24-CEN-319 is a digitoxigenin based linker-ready agent that comprises a steroid, a linker and an active maleimide group. The general synthetic steps for the preparation of PEG24-CEN-319 are as follows.

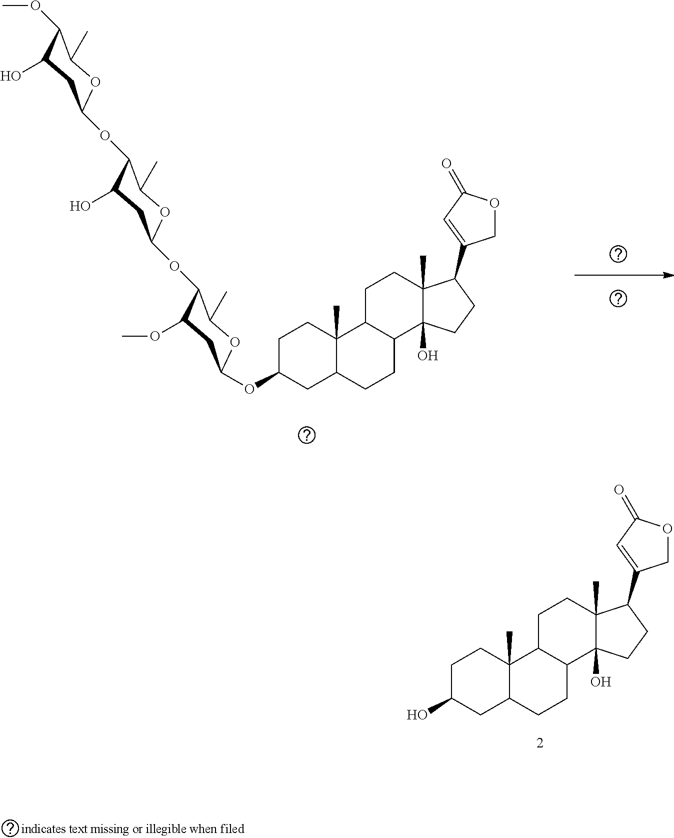

### Digitoxigenin (2)

To a suspension of digitoxin (1, 10.2 g, 13.33 mmol) in MeOH (270 ml) at RT was added PTSA (0.25 g, 1.33 mmol). The reaction mixture was stirred at RT for 2 days. The solvent was removed in vacuo. The crude material was purified by flash chromatography (silica gel, Hexanes-EtOAc 4:6 to 3:7) to give 2 as a white solid (3.44 g, 68%). ¹H-NMR (300 MHz, DMSO-d₆) 5.90 (s, 1H, H-22), 4.97 (dd, J=1.5, 18.4 Hz, 1H, H-21), 4.87 (dd, J=18.2, 1.6 Hz, 1H, H-21), 4.17 (d, J=3.0 Hz, OH), 4.05 (s, OH), 3.89 (m, 1H, H-3), 2.75-2.70 (m, 1H, H-17), 2.09-1.97 (m, 2H), 1.84-1.70 (m, 5H), 1.64-1.56 (m, 2H), 1.49-1.30 (m, 8H), 1.20-1.05 (m, 4H), 0.87 (s, 3H), 0.76 (s, 3H); ¹³C-NMR (75 MHz, DMSO-d₆) 176.3, 173.8, 116.2, 83.8, 73.1, 64.6, 50.2, 49.4, 40.9, 39.0, 35.7, 35.0, 34.7, 33.1, 32.2, 29.5, 27.5, 26.5, 26.4, 23.7, 21.1, 20.8, 15.7.

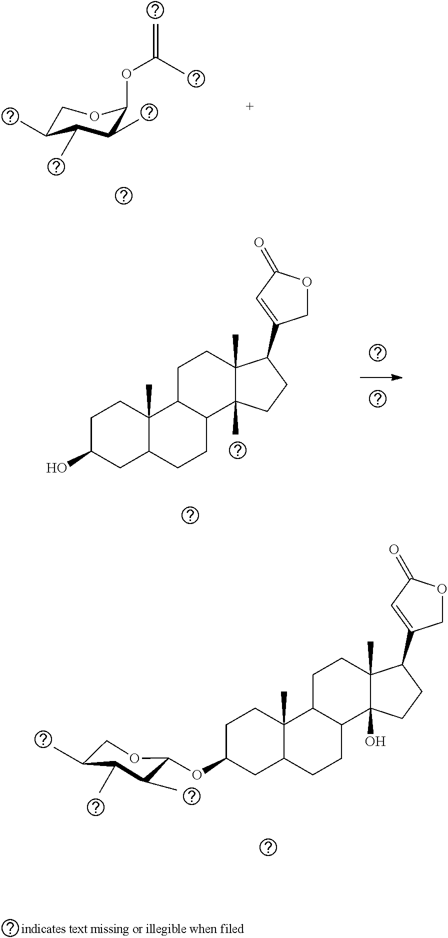

### Digitoxigenin-2,3-di-O-benzoyl-4-deoxy-4-azido-β-L-xylopyranoside (4)

A solution of 3 (1.40 g, 2.67 mmol) in freshly distilled dry CH₂Cl₂ (5 mL) and digitoxigenin (2, 1.00 g, 2.67 mmol) were added to a suspension of activated 4 Å molecular sieves (0.3 g) in freshly distilled CH₂Cl₂ (5 mL) at 0° C. under argon. After 10 min of stirring, TMSOTf (24 μL, 0.134 mmol) was added. The reaction mixture was stirred at 0° C. for 2 hours, then quenched with Et₃N (40 μL, 0.268 mmol). The solvent was removed in vacuo. The crude material was purified by flash chromatography (silica gel, Hexanes-EtOAc 7:3 to 1:1) to give 4 as a white powder (1.62 g, 82%). ¹H-NMR (300 MHz, CDCl₃) 8.00-7.94 (m, 4H), 7.56-7.48 (m, 2H), 7.42-7.34 (m, 4H), 5.84 (br s, 1H, H-22), 5.52 (dd, J=8.8 Hz, 1H, H-3′), 5.32 (dd, J=8.8 Hz, 6.8 Hz, 1H, H-2′), 4.97 (dd, J=18.2, 1.9 Hz, 1H, H-21), 4.78 (dd, J=1.7, 18.2 Hz, 1H, H-21), 4.70 (d, J=6.9 Hz, 1H, H-1′), 4.20 (dd, J=12.0, 4.9 Hz, 1H, H-5′), 4.03 (m, 1H, H-3), 3.86 (ddd, J=9.2, 9.1, 4.9 Hz, 1H, H-4′), 3.47 (dd, J=12.0, 9.6 Hz, 1H, H-5′), 2.76-2.71 (m, 1H, H-17), 2.18-2.02 (m, 2H), 1.89-1.63 (m, 6H), 1.55-1.25 (m, 10H), 1.20-1.01 (m, 4H), 0.81 (s, 3H), 0.57 (s, 3H); ¹³C-NMR (75 MHz, CDCl₃) 174.7, 174.6, 165.7, 165.2, 133.6, 133.4, 130.0, 129.9, 129.4, 129.0, 128.6, 128.5, 117.7, 98.9, 85.6, 73.7, 73.5, 72.9, 71.4, 63.1, 59.2, 51.0, 49.7, 41.9, 40.0, 36.3, 35.7, 35.0, 33.2, 33.1, 29.5, 27.0, 26.5, 23.9, 23.3, 21.4, 21.2, 15.8. ESI-MS (m/z): calcd for C₄₂H₄₉N₃O₉ [M+H]⁺ 740.3, found 740.3, [M+NH₄]⁺ 757.4, found 757.5, [M+Na]⁺ 762.3, found 762.4.

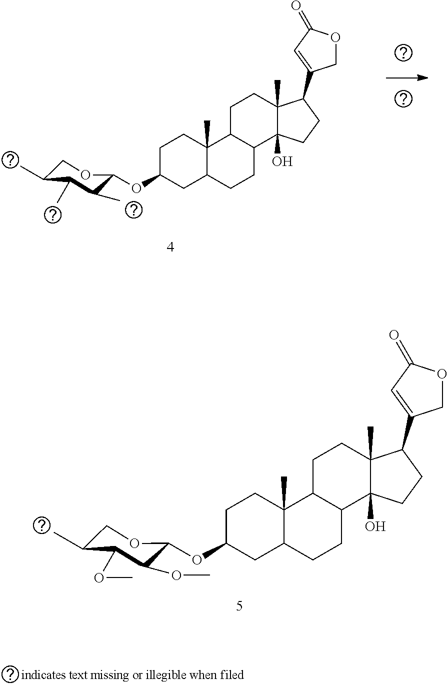

### Digitoxigenin-4-deoxy-4-azido-13-L-xylopyranoside (5)

To a solution of 4 (500 mg, 0.676 mmol) in MeOH (5 mL) was added a saturated aquous solution of Na₂CO₃ (0.5 mL) at RT. The reaction mixture was stirred at RT for 3 days. The pH was adjusted to 5 with 1N HCl. CH₂Cl₂ (15 ml) was added. The organic layer was washed with water (2×5 mL) and brine (5 mL), dried (Na₂SO₄) and concentrated in vacuo. The crude material was purified by flash chromatography (silica gel, Hexanes-EtOAc 6:4 to 4:6) to give 5 as a white foam (262 mg, 73%). ¹H-NMR (300 MHz, DMSO-d₆/D₂O) 5.89 (br s, 1H, H-22), 4.96 (dd, J=18.2, 1.2 Hz, 1H, H-21), 4.86 (dd, J=18.5, 1.4 Hz, 1H, H-21), 4.14 (d, J=7.7 Hz, 1H, H-1′), 3.86 (m, 1H, H-3), 3.74 (dd, J=11.3, 5.2 Hz, 1H, H-5′), 3.39 (dd, J=10.2, 4.9 Hz, 1H, H-4′), 3.27 (dd, J=9.1 Hz, 1H, H-3′), 3.08-2.97 (m, 2H, H-2′, H-5′), 2.74-2.70 (m, 1H, H-17), 2.08-1.96 (m, 2H), 1.87-1.56 (m, 8H), 1.49-1.27 (m, 8H), 1.15-1.07 (m, 3H), 0.86 (s, 3H), 0.75 (s, 3H); ¹³C-NMR (75 MHz, DMSO-d₆/D₂O) 176.3, 173.8, 116.2, 101.6, 83.8, 75.5, 73.6, 73.1, 72.9, 62.9, 61.4, 50.2, 49.4, 40.9, 39.0, 35.9, 34.8, 34.8, 32.2, 31.6, 29.5, 26.4, 23.7, 23.5, 21.0, 20.8, 15.7. ESI-MS (m/z): calcd for C₄₂H₄₉N₃O₉ [M+H]⁺ 532.3, found 532.2, [M+NH₄]⁺ 549.3, found 549.3, [M+Na]⁺ 554.3, found 554.2.

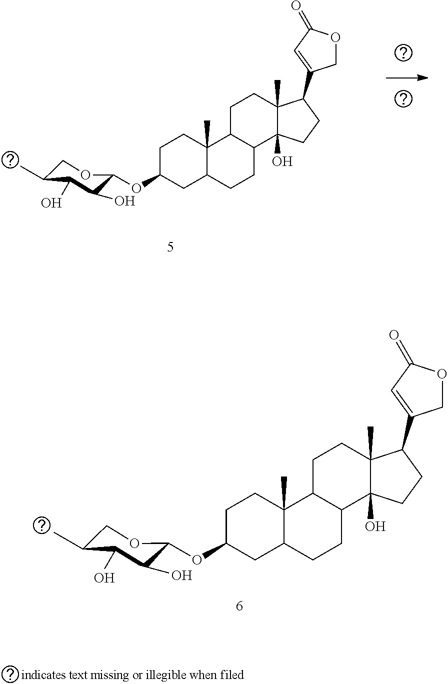

### Digitoxigenin-4-deoxy-4-amino-13-L-xylopyranoside (6)

To a solution of 5 (230 mg, 0.433 mmol) in THF—H₂O (8 mL, 90:10) was added PPh₃ (567 mg, 2.16 mmol). The reaction mixture was stirred overnight at 40° C. The solvent was removed in vacuo. The crude material was purified by flash chromatography (silica gel, CHCl₃-MeOH—H₂O 85:15:1 to 75:25:2.5) to give 6 as a white powder (160 mg, 73%). Rₜ 12.67 min (Gemini C18, 5 μm, 4.6 mm×250 mm, 10% to 90% ACN, 0.1% TFA, over 18 min, 1 mL.min⁻¹). ¹H-NMR (300 MHz, DMSO-d₆/D₂O) 5.89 (br s, 1H, H-22), 4.95 (dd, J=18.5, 1.5 Hz, 1H, H-21), 4.86 (dd, J=18.4, 1.4 Hz, 1H, H-21), 4.09 (d, J=7.1 Hz, 1H, H-1′), 3.86 (m, 1H, H-3), 3.62 (dd, J=11.4, 5.0 Hz, 1H, H-5′), 2.98-2.89 (m, 3H, H-2′, H-3′, H-5′), 2.74-2.70 (m, 1H, H-17), 2.54-2.46 (m, 1H, H-4′), 2.08-1.96 (m, 2H), 1.87-1.56 (m, 8H), 1.49-1.27 (m, 8H), 1.15-1.02 (m, 3H), 0.85 (s, 3H), 0.75 (s, 3H); ¹³C-NMR (75 MHz, DMSO-d₆/D₂O) 176.4, 173.8, 116.2, 109.3, 101.9, 83.8, 77.4, 73.6, 73.1, 72.5, 66.6, 53.1, 50.2, 49.4, 41.0, 39.0, 36.0, 34.9, 34.8, 32.2, 31.7, 29.6, 26.4, 23.7, 23.5, 21.0, 20.8, 15.7. ESI-MS (m/z): calcd for C₂₉H₄₁NO₇ [M+H]⁺ 506.3, found 506.4.

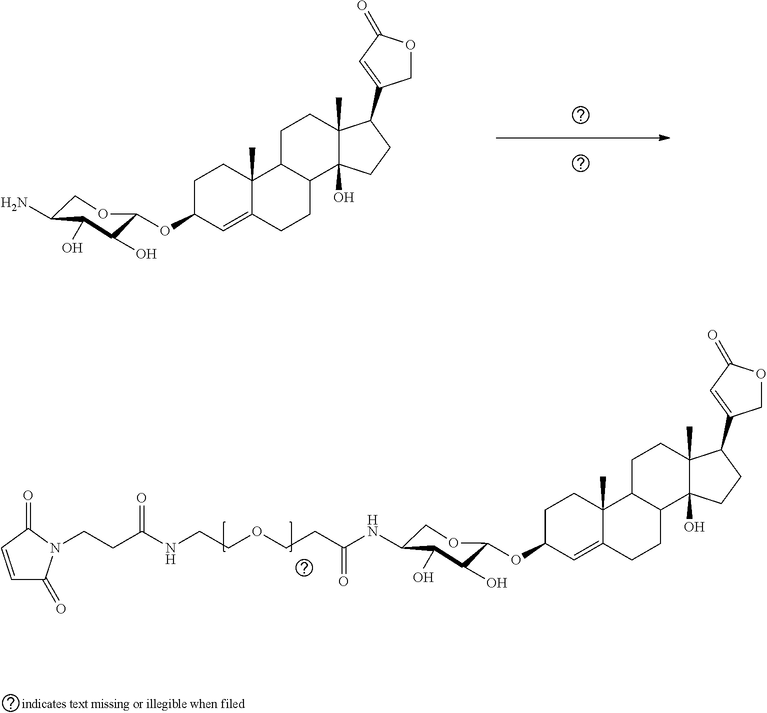

PEG24-CEN-319.

To a solution of Digitoxigenin-4-deoxy-4-amino-β-L-xylopyranoside (22 mg, 0.043 mmol) and maleimide-PEG₂₄-NHS ester (60 mg, 0.043 mmol) in N,N-dimethylacetamide (0.4 mL) was added Et₃N (0.03 mL, 0.215 mmol). The reaction mixture was stirred at RT for 2 hours and then purified by HPLC to give 7 as a colorless oil (56 mg, 73%). Rₜ 19.32 min (Gemini C18, 5 μm, 4.6 mm×250 mm, 20% to 50% over 18 min, then to 90% over 2 min, ACN, 0.1% TFA, 1 mL.min⁻¹). ESI-MS (m/z): calcd for C₈₆H₁₄₉N₃O₃₅ [M+Na]⁺ 1806.987, found 1806.949, [M+K]⁺ 1822.961, found 1822.922.

### Example 2

### M53 Antibody; Epitope Mapping; and Conjugation to Linker-Ready Agent

The M53 antibody (described in Shimamura et al. J. Clinical Oncology 21(4) 659-667 (2003)) and the control 4F12 antibody are used in these examples. Antibody M53 recognizes and binds human dysadherin; amino acid sequence shown in SEQ ID NO. 1), and control antibody 4F12 recognizes and binds a peptide found within SEQ ID NO. 1 but not to dysadherin as expressed on the surface of human cells. Both antibodies described are monoclonal of mouse origin and of the IgG1 kappa isotype form.

Overlapping peptide sequences of 15-17 amino acids in length (SEQ ID NO. 2 through SEQ ID NO. 16; termed CENP001, and CENP004-CENP017, respectively) were synthesized and correspond to positions 24 through 145 of the extracellular domain (residues 22-145 of SEQ ID NO: 1) of dysadherin. Each peptide contained a C-terminal cysteine to facilitate conjugation to maleimide activated BSA (cat. number: 77116, Pierce Biotechnology, Rockford, Ill.). Peptides and L-cysteine (used as a BSA-control conjugate) were coupled to BSA per the manufacturer's protocol. Each well of clear 96 well medium bind ELISA plates (cat. number: 9017, Corning, Corning, N.Y.) were individually coated with 250 ng of BSA-peptide or BSA-control conjugate in 100 uL of 200 mM carbonate buffer, pH 9.6 overnight at 4° C. Coated ELISA plates were washed (×3) with PBS pH 7, blocked 30 min with PBS pH 7 containing 1% NFDM (PBS+NFDM), and then washed (×3) with PBS, pH 7. M53 diluted to 1 ug/mL (100 uL) in PBS+NFDM was added to all BSA-peptide and BSA-control-conjugate coated wells, incubated 30 min at RT, and the wells washed (×3) with PBS, pH 7. Goat anti-mouse IgG alkaline phosphatase (cat. number: A1418, Sigma-Aldrich, St. Louis, Mo.) was diluted 1:15,000 in PBS+NFDM and added (100 uL) to each well, incubated 30 min at RT, and the wells washed (×3) with PBS, pH 7. PNPP at 1 mg/mL in 1M DEA with 50 mM MgCl₂, pH 9.8 was added (100 uL per well), incubated 30 minutes at RT and the absorbance at 405 nm was determined using a Wallac Victor² Model 1420-041 assay plate reader (Perkin Elmer, Gaithersburg, Md.).

It was determined that M53 bound two and overlapping peptide sequences (SEQ ID NO. 4 and SEQ ID No. 5) revealing the epitope to reside between positions 38 to 59 of human dysadherin (SEQ ID NO. 1). Given this, a peptide CENP018 (SEQ ID NO. 17) was produced whose sequence was a hybrid of the overlapping petides (SEQ ID NO. 4 and SEQ ID No. 5). Using the methods of this example it was determined that M53 binds the peptide sequence (SEQ ID. 17). In various embodiments of the Class 1 EDCs of the invention, the antibody binds to this epitope.

Preparing Antibodies for Conjugation.

Selective conjugation of antibodies through sulfhydryl residues first requires antibody disulfide reduction. This was carried out using one of two methods. In one method, termed BME, 2-Mercaptoethanol was used, and for the second method, termed TCEP, tris(2-carboxyethyl)phosphine was used. Each is described below and was used in various examples to prepare antibodies for coupling, although the TCEP method was more generally employed.

For antibody reduction using the BME method, 1 mg 13-ME is added to 1 mg of antibody and mixed in 500 ul 0.1M sodium phosphate, 0.15 M NaCl, 5 mM EDTA and incubated at 37° C. for 1.5 hr. Excess f3-ME is removed by gel filtration using Sephadex G-25 or similar. The final solution is brought to 1 ml in PBS.

For antibody reduction using the TCEP method, each antibody was treated with 8 molar equivalents of tris(2-carboxyethyl)phosphine (TCEP) (cat. number: HR2—651, Hampton Research) in 20 mM sodium phosphate pH 7, 150 mM NaCl, and 1 mM diethylenetriamine pentaacetic acid (DTPA) (MP Biomedical LLC) for 2 h at 37° C. Reactions were placed in an ice bath, and once cooled, linker-agents were added.

Coupling Linker-Agent to Antibody.

To cold reduced antibody, 9.6 molar equivalents of linker-ready agents per equivalent antibody were added. The reactions were allowed to proceed for 30 min. on ice. L-Cysteine was then added at a 2-fold excess to quench any unreacted maleimide groups. To concentrate the agent conjugates and remove excess linker-ready agent that was not coupled to antibody, the conjugation reactions were concentrated then buffer exchanged 3× for 20 mM sodium phosphate pH 7 and 150 mM NaCl using Microcon Ultracel YM-30 (Millipore) 30K cutoff spin concentration devices. Capped linker-ready agent controls (no antibody) were made following the above procedure minus antibody addition and concentration steps.

Determination of Drug Loading.

For antibody drug conjugates that contained the steroid scillarenin, drug loading was estimated using absorbance. First, the absorbance of free antibody was measured at both 280 nm (A₂₈₀Ab) and 299 nm (A₂₉₉Ab) to determine antibody constant [Constant Ab]. Next, the absorbance of free drug was measured at both 280 nm (A280drug) and 299 nm (A₂₉₉drug) to determine drug constant [Constant Drug]. Finally, the absorbance of antibody drug conjugate was measured [A₂₈₀ and A₂₉₉]. Concentration conversions were based upon an antibody molar extinction coefficient at 280 nm (204,000 M⁻¹ cm⁻¹) and drug molar extinction coefficient at 299 nm (5623 M⁻¹ cm⁻¹). The following calculations were employed to estimate drug loading: [Constant Ab]=A₂₉₉Ab/A₂₈₀Ab; [Constant Drug]=A₂₉₉drug/A₂₈₀drug; A₂₈₀Ab=A₂₈₀−(A₂₉₉−[Constant Ab]×A₂₈₀)/([Constant drug]−[Constant Ab]); A₂₉₉drug=A₂₉₉−[Constant Ab]×A₂₈₀Ab; Antibody concentration=A₂₈₀Ab/204,000 M⁻¹ cm⁻¹; Drug concentration=A₂₉₉drug/5623 M⁻¹ cm⁻¹; Drug loading=drug concentration/antibody concentration. For antibody drug conjugates that contained the steroid digitoxigenin, drug loading could not be estimated by absorbance, because it does not contain a chromophore that absorbs light in the visible range, and so was estimated (based on loading efficiency of scillarenin containing conjugates and relative activities). Typically, in cancer cell based in vitro assays, higher drug loading provided more cytotoxic conjugates.

### Example 3

### In Vitro Cytotoxicity Assays

This example demonstrates that illustrative compounds of the invention are useful at targeting and killing tumor cells in vitro. Antibody M53 (specific for dysadherin expressed on human cell lines H460, A549, A375, PANC1, and H929 [but not H520]) and antibody 4F12 (specific for a peptide sequence within the dysadherin extracellar portion but does not recognize dysadherin expressed on human cell lines) were used to produce drug conjugates as described in Example 2. EDCs were constructed using linker-ready agents PEG24-CEN09-106, PEG24-CEN09-107, PEG24-CEN10-110 and PEG24-CEN-319. As described in Example 1, linker-ready agent PEG24-CEN-319 contain digitoxigenin while the others contain scillarenin. Linker-ready agents PEG24-CEN09-106, PEG24-CEN09-107, PEG24-CEN10-110 all use different linkers. Specifically, PEG24-CEN09-106 and PEG24-CEN09-107 have different sugars in the linker, while PEG24-CEN10-110 contains a longer linker that includes a primary amine. These linker-ready agents along with antibodies M53 and 4F12 were used to produce active EDCs M53-PEG24-CEN09-106, M53-PEG24-CEN09-107, M53-PEG24-CEN10-110 and M53-PEG24-CEN-319 and non-active control conjugates 4F12-PEG24-CEN09-106 and 4F12-PEG24-CEN10-110 (linker-ready agents were also used as controls).

In Vitro Cancer Cell Cytotoxicity Analysis.

All cell lines were maintained in complete media [RPMI medium 1640 supplemented with 10% (wt/vol) fetal bovine serum and gentamycin (50 μg/ml)]. Cells were plated at a density of 1250 cells per well of each 384-well white tissue culture treated microtiter plate in 20 uls complete media, and then were grown for 24 hour at 37° C. with 7% CO₂ in a humidified incubator before addition of test compound. In a separate 96-well plate, M53, capped linker-ready agents and antibody conjugates (in PBS) stocks were serially diluted in complete media at 5× final working concentrations, and 5 ul added to the cells used in the assay. Cells were incubated with the agent/conjugate for 3 days before cell viability testing. Cell viability testing used the CellTiter-Glo luminescent cell viability assay (Promega, Madison, Wis.). ED50 values of the agents to each cell line were determined using GraphPad Prism 5 software.

The results are shown in the Table below and demonstrate that the different conjugates of the invention that contain M53 are cytotoxic at picomolar to low nanomolar concentrations in those cell lines that present the target of the antibody and drug in close proximity. All of the cell lines below, except H520, express the Na,K-ATPase with the dysadherin subunit and so present the target of the antibody and drug in close proximity. The H520 cell line expresses the target of the drug but contains a different gamma subunit isotype and so does not present the target of the antibody. The results also show that the capped linker-ready agents and the 4F12 conjugates are at least 100-fold less active when compared to the M53-based Class 1 EDCs. The results also show that the antibody M53 alone is inactive at the highest concentrations tested, thus demonstrating that the antibody requires the steroid drug to exhibit cytotoxicity. The results also show that M53-PEG24-CEN09-106 is not active on H520 cells, which have been shown by immunohistochemistry not to express the M53 antibody's target dysadherin (FXYD5).

| | CELL LINE | | | | | |
|---|---|---|---|---|---|---|
| | H460 | H520 | A549 | A375 | PANC | H929 |
| FXYD5 Cell Surface Expression | ++ | −− | ++ | ++ | ++ | ++ |
| M53 | | | >5000 | >2000 | >5000 | >200 |
| M53-PEG24-CEN09-106 | 0.3 | >50 | 0.2 | 0.4 | 0.3 | 0.1 |
| M53-PEG24-CEN09-107 | | | 0.5 | 2.6 | | |
| M53-PEG24-CEN10-110 | 0.2 | | 0.1 | 0.5 | | |
| M53-PEG24-CEN-319 | | | 1.6 | | >200 | >200 |
| 4F12-PEG24-CEN09-106 | 142 | 162 | 85 | 280 | 78 | 266 |
| 4F12-PEG24-CEN10-110 | 65 | | 100 | 184 | | |
| PEG24-CEN09-106 | 77 | 40 | 31 | 66 | 27 | 90 |
| PEG24-CEN09-107 | | | 37 | 127 | | |
| PEG24-CEN10-110 | 40 | | 31 | >50 | 38 | |
| PEG24-CEN-319 | | | 348 | | 542 | |

The table above shows EC50 values in nanomolar of drug antibody linker conjugates, M53 antibody alone and linker-ready drug agents.

In Vitro Cancer Cell Cytotoxicity Analysis Using M53-PEG24-CEN09-106 Drug Combinations.

The cytotoxic effects against the NSCLC cell line A549 of M53-PEG24-CEN09-106 in combination with the FGFR kinase inhibitor PD 173074 (LC Laboratories, Woburn, Mass.) or a soluble recombinant human TRAIL (izTrail, Enzo Life Sciences, Farmingdale, N.Y.) or Everolimus or CEN10-128-cys (Centrose patent application US 2011/0064752 A1) were measured. The studies employed a fixed ratio of the drugs (based on the EC50 values of each alone) across a concentration gradient. Single drug ED50 values were first determined and from those values combination analysis were prepared by 3-fold serial dilutions in 8 steps for all compounds. Combinations were tested using fixed concentration ratios. For PD 173074 and M53-PEG24-CEN09-106, the ratio gradient went from 0.12 microM and 2 picoM to 251 microM and 3600 picoM respectively. For TRAIL and M53-PEG24-CEN09-106, the ratio gradient went from 0.25 ng/mL to 2 picoM up to 540 ng/mL to 3600 picoM respectively. For Everolimus and M53-PEG24-CEN09-106, the ratio gradient went from 0.01 nanoM and 0.4 picoM to 26.1 nanoM to 900 picoM respectively. For CEN10-128-cys and M53-PEG24-CEN09-106, the ratio gradient went from 160 nanoM and 1 picoM to 330 microM to 1.7 nanoM respectively. All compound concentrations and combinations were tested in duplicate. Cell culture and cell viability assays were performed as described.

| Drugs | EC50 | |
|---|---|---|
| M53-PEG24-CEN09-106 | 0.3 | nM |
| PD 0173074 | 29 | uM |
| izTrail | 82 | ng/mL |
| Everolimus | 11 | nM |
| CEN10-128-cys | 60 | uM |

| | |
|---|---|
| PD 0173074 + M53-PEG24-CEN09-106 | 13 uM + 0.2 nM |
| izTrail + M53-PEG24-CEN09-106 | 36 ng/mL + 0.2 nM |
| Everolimus + M53-PEG24-CEN09-106 | 2 nM + 0.06 nM |
| CEN10-128-cys + M53-PEG24-CEN09-106 | 13 uM + 0.06 nM |

The table above shows EC50 values of the drugs (alone and in combination) when tested on A549 cells grown in culture.

The data in the Table above shows that the EC50 values for the drugs in combination are less than that of the drugs by themselves and so show that these combinations are synergistic. The results demonstrate that TRAIL and fibroblast growth factor receptor kinase inhibitors, mTOR inhibitors and glycolysis inhibitos work in synergy with Class 1 EDCs to promote cancer cell death in a dose dependent manner. In addition, when in combination, the studies should that total surviving cells decreased when compared to M53-PEG24-CEN09-106 alone. In addition, when in combination, the studies showed that total surviving cells decreased when compared to M53-PEG24-CEN09-106 alone. This indicates that these combinations should also lead to increased tumor suppression and/or tumor regression in vivo.

In vitro normal cell cytotoxicity. The in vitro cytotoxic activity of M53-PEG24-CEN09-106, PEG24-CEN09-106, M53-PEG24-CEN-319, PEG24-CEN-319, proscillaridin and digitoxin against dysadherin positive primary normal human cells and a dysadherin positive human non-small cell lung carcinoma (NSCLC) cell line were tested and compared. The cells used were primary human renal epithelial cells (HREpC), primary human umbilical vein endothelial cells (HUVEC), primary human umbilical artery endothelial cells (HUAEC), and the A549 NSCLC cells. The primary cells were obtained from PromoCell GmbH, Heidelberg, Germany, and the NSCLC cell line A549 was obtained from ATCC. Primary renal epithelial cells were grown in Renal Epithelial Cell Growth Medium 2 (PromoCell GmbH, Heidelberg, Germany). Primary endothelial cells were grown in Endothelial Cell Growth Medium 2 (PromoCell GmbH, Heidelberg, Germany). The NSCLC cell line was grown in RPMI-1640 (HyClone, Thermo Scientific) supplemented with 10% fetal bovine serum. HREpC, HUVEC, HUAEC, and A549 cells were plated in 384 well plates at 1250, 1875, 2500, and 1250 cells/well (respectively) and allowed to incubate for 24 hrs at 37° C., 5% CO2, and 100% humidity. Various concentrations of conjugates M53-PEG24-CEN09-106 and M53-PEG24-CEN-320 and PEG24-CEN09-106 and PEG24-CEN-320 were added to the wells in a total volume of 5 uL of media and the plates were incubated for an additional 72 hrs. After 72 hrs of exposure to agent, cell viability was evaluated using the Cell Titer-Glo Luminescent Cell Viability Assay (Promega, Madison, Wis.) according to the manufacturer's instructions. Luminescence measurements were performed using a Wallac Victor² Model 1420-041 assay plate reader (Perkin Elmer, Gaithersburg, Md.). EC₅₀ values for each test agent were determined using GraphPad Prism 5 software. The concentrations at which compounds exert a half maximal effect on cell viability on the respective cell lines are shown in the table below, the EC50 concentrations indicated for PEG24-CEN09-106 and PEG24-CEN-320 are the concentration of small drug molecule itself and the EC50 concentrations indicated for M53-PEG24-CEN09-106 and M53-PEG24-CEN-320 are the concentration of the antibody portion of these conjugates. These data illustrate that M53-PEG24-CEN09-106 is significantly less cytotoxic, 170 to >1180 times, against FXYD5 positive primary normal cells relative to the FXYD5 positive NSCLC cell line A549.

| | CELL LINE | | | |
|---|---|---|---|---|
| | A549 | HUVEC | HUAEC | HREpC |
| FXYD5 Cell Surface Expression | ++ | + | + | + |
| M53-PEG24-CEN09-106 | 0.2 | 30 | 29 | >200 |
| PEG24-CEN09-106 | 32 | 37 | 32 | |
| Proscillaridin | 2.1 | 5.6 | 5.6 | 10 |
| M53-PEG24-CEN-319 | 1.6 | >200 | >200 | |
| PEG24-CEN-319 | 348 | >200 | >200 | |
| Digitoxin | 17 | 39 | 47 | 135 |

### Example 4

### M53 Sequencing and Production of Human Chimeric Antibody

mRNA isolated from a hybridoma cell line that produces M53 was cloned and sequenced to determine the nucleic acid sequences that code for the variable domains of this mouse IgG1, kappa immunoglobulin. 5′-RACE (Smart RACE kit; Clontech) was used to amplify the 5′ of mRNA encoding the IgG heavy and kappa light chains of M53. Briefly, about 1 μg of mRNA is used for reverse transcription to produce cDNA pools. Next, cDNA was amplified via PCR with a universal primer provided with the RACE kit and gene specific primers. The universal primer was SEQ ID NO: 18, and the gene specific primers for IgG1/IgG2A and IgG2b were SEQ ID NO: 19 and SEQ ID NO: 20, respectively. PCR products were gel purified and cloned into pSUPER-blunt vector (Adexon) and multiple colonies sequenced. Endogenous aberrant light chain was removed by screening and only non-aberrant clones were sequenced. Sequencing results were analyzed on NTI vector. Results of sequencing analysis of all clones revealed that the hybridoma produces a true monoclonal antibody. The coding sequence of the M53 heavy chain variable region is shown in nucleic acids (240-599) of SEQ ID NO: 21, and the coding sequence of the M53 light chain variable region is shown in nucleic acids (221-559) of SEQ ID NO: 22

Chimeric mouse-human monoclonal antibodies (i.e., chimeric antibodies) can be produced by recombinant DNA techniques known in the art (Current Opinion in Biotechnology 12, no. 2, Apr. 1, 2001: 188-194). For example, the use of mammalian expression vectors that code for the heavy chain (HC) and light chain (LC) proteins of an assembled IgG molecule that comprise variable regions derived from mouse IgG fused to constant regions of human IgG are well known in the art. The HC and LC variable region encoding DNA isolated from the M53 producing hybridoma line were used to construct mammalian expression vectors for the production of chimeric M53 (cM53), except the heavy chain variable domain coding sequence of SEQ ID NO. 21 (nucleotides 243 to 245) was altered to encode a cysteine instead of a valine at position 2 of the chimeric heavy chain protein (SEQ ID NO: 23). Standard recombinant DNA techniques (BMC Biotechnol. 2006 Dec. 22; 6:49) were employed to produce chimeric M53 (cM53) mammalian expression vectors for the expression of HC (SEQ ID NO: 23) and LC (SEQ ID NO: 24) protein sequences whose variable regions, derived from the IgG1 kappa antibody M53, were fused to human IgG1 HC and LC constant regions. The DNA sequence of the vector insert for the expression the chimeric HC (SEQ ID NO: 25) codes for a signal peptide (nucleotides 1 to 90), the M53 variable HC domain (nucleotides 91 to 450, where nucleotides 94 to 96 are altered to encode for cysteine instead of valine), and a human IgG1 constant HC domain (nucleotides 451 to 1443). The DNA sequence of the vector insert for the expression the chimeric LC (SEQ ID NO: 26) codes for a signal peptide (nucleotides 1 to 60), the M53 variable LC domain (nucleotides 61 to 399), and a human IgG1 constant LC domain (400 to 723). The signal peptide sequences (SEQ ID NO: 27 and 28) used for transient expression of cM53 in HEK293 cells are cleaved from and allow for the secretion of cM53 in the culture media, other signal peptide sequences are known in the art that would function as well (Trends in Cell & Molecular Biology (2007) 2, 1-17). The HC and LC mammalian expression vectors were transiently expressed in HEK293F cells (Invitrogen). Briefly, plasmid DNA (a 1:1 mixture of HC and LC mammalian expression vectors) was combined 1:4 with polyethylenimine (i.e. for 1 liter of HEK293F cell culture, 1 mg of plasmid DNA and 4 ml of polyethylenimine (1 mg/ml) were mixed in 50 ml of OptiMEM (Invitrogen), then incubated for 10 min. before adding to cells). Cells were cultured at 37° C., 8% CO2, with shaking (125 rpm) for 5-7 days and cell culture media was collected when cell viability approached 50%. The resulting cell culture media was centrifuged and the supernatant was filtered with a 0.22 um filter. Chimeric mouse/human IgG was isolated from the filtered cell culture media by protein G affinity chromatography.

Purified cM53 and M53 antibodies were conjugated to the linker-ready reagent PEG24-CEN09-106. Briefly, the antibodies were treated with 10 equivalents of dithiothreitol (DTT) 1 hr at room temperature (RT). Excess DTT was removed by buffer exchange for 20 mM sodium phosphate pH 7 and 150 mM NaCl using Microcon Ultracel YM-30 (Millipore) 30K cutoff spin concentration devices. The antibodies were treated with 2 equivalents of dehydroascorbic acid for 3 hours at RT, then 5 equivalents of PEG24-CEN09-106 was added to each reaction and incubated 1 hour at RT. L-Cysteine was then added at a 2-fold excess to PEG24-CEN09-106 to quench any unreacted maleimide groups. To achieve sample concentration and removal of excess linker-agent that was not coupled to antibody, the conjugation reactions were concentrated and buffer exchanged 3× for 20 mM sodium phosphate pH 7 and 150 mM NaCl using Microcon Ultracel YM-30 (Millipore) 30K cutoff spin concentration devices.

The resulting conjugates were tested for in vitro activity against the NSCLC cell line A549 as described above. The resulting conjugates, cM53-PEG24-CEN09-106 and M53-PEG24-CEN09-106, showed similar activity against A549 cells (EC50 values of 2.0 and 1.9 nM, respectively).

Single chain variable fragment construction. A synthetic gene construct was designed that codes for an scFv containing the heavy and light variable chains of M53 where a cysteine was substituted for valine at the penultimate amino acid position at the N-terminus of the heavy variable chain (SEQ ID NO: 29). An expression construct (pJexpress411:58866-CENscFv003[Cysteine on heavy chain] optEc V2) containing this scFv gene construct was synthesized by DNA 2.0 (Menlo Park, Calif.). When expressed in E. coli this construct produces a protein (SEQ ID NO: 30) that comprises the PhoA (alkaline phosphatase) signal sequence that targets the expressed protein to the periplasm where it is released from the rest of the protein (positions 1 to 21), the M53 variable HC domain (position 22 to 141, where position 23 is a cysteine substituted for valine), a glycine rich flexible linker (positions 142 to 156), and the M53 variable LC domain (positions 157 to 269).

The scFv003 expression construct was transformed into E. coli EXPRESS BL21(DE3) chemically competent cells (Lucigen Corporation, Middleton, Wis.). Six liters of LB medium containing 30 ug/mL Kanamycin was inoculated with the above transformant and grown to 0.8 O.D. 600 at 17° C. when IPTG was added at 200 uM. Following an 18 hour induction the 20.6 grams of cell paste was collected by centrifugation of the growth media at 4000 rpm. The cells were lysed on ice in 90 mL of PBS pH7.4 plus 1 mM DTPA by sonication. The lysate was clarified by centrifugation at 14,000 rpm for 20 minutes then filtered through a 0.8 then 0.45 um filter. The anti-FXYD5 scFv003 was purified from the lysate by immunoaffinity chromatography. The immunoaffinity resin was prepared by conjugation of CENP018 (PTRAPDAVYTELQC) (SEQ ID NO: 11) to SulfoLink Coupling Resin (Pierce Biotechnology, Rockford, Ill.) at loading ratio of 1 mg of peptide/mL of resin following the manufactures protocol. A column containing 2 mL of peptide modified resin was prepared and the lysate was passed over this resin. The column was then washed with 100 column volumes of PBS pH7.4 plus 1 mM DTPA and the bound scFv eluted with 0.1M citric acid, protein containing eluate was immediately neutralized with Tris base. The protein containing eluate was concentrated and buffer exchanged for PBS pH7.4 plus 1 mM DTPA using a 5 mL 6K MWCO polyacrylamide desalt column (Pierce Biotechnology, Rockford, Ill.).

The unpaired cysteine of the scFv protein prepared above was reacted with PEG₂₄-CEN09-106 without the need of cysteine disulfide reduction. Briefly, 5 molar equivalents of PEG₂₄-CEN09-106 was added to an ice chilled 32 uM solution of scFv003 in PBS pH 7.4÷1 mM DTPA and allowed to react overnight on ice. Unreacted maleimide was quenched by the addition of 7.5 molar equivalents of L-cysteine and allowed to react 30 minutes at room temperature. The resulting conjugates were then purified from excess linker-drug by repeated buffer exchange using Amicon Ulta-0.5 mL 10K centrifugal concentrators (Millipore, Billerica, Mass.). PEG₂₄-CEN09-106 loading was determined for this conjugate as in example 2 using a molar extinction coefficient value 51,590 M⁻¹ cm⁻¹@280 nm for scFv.

The resulting conjugate was tested for in vitro activity against the NSCLC cell line A549 as described above. The resulting conjugates showed activity against A549 cells (EC50=1.6 nM). In addition to this result for scFv-PEGn-CEN09-016 (where n=24), scFv-PEGn-CEN09-016 conjugates where n=12 and 36 were also produced and tested against A549. The scFv-PEG₁₂-CEN09-016 and scFv-PEG₃₆-CEN09-016 were determined have 1 drug per scFv and had EC50 values of 9.1 and 1.1, respectively. Additional tests it was shown that the cytotoxic activity of scFv-PEG₂₄-CEN09-106 can be neutralized or competed with M53 antibody, demonstrating the scFv and M53 react with the same epitope.

### Example 5

### Pharmacokinetics of M53 and M53-PEG24-CEN09-106 with Varying Drug Loading

To evaluate pharmacokinetic effects of drug loading, unconjugated antibody M53 and antibody drug conjugate M53-PEG24-CEN09-106 formulations with a drug loading of 2, 5, and 9 drugs per antibody were administered to SCID beige mice (Harlan Laboratories, n=2) at 1 mg/kg of test material (based on the antibody component) by tail vein injection. Serum was isolated from blood samples taken by retro-orbital blood collection at 1 hour, and 1, 2, 3, 6, and 15 days post-injection. Blood was collected into heparin coated tubes followed by centrifugation (5,000×g, 5 minutes) to isolate plasma.

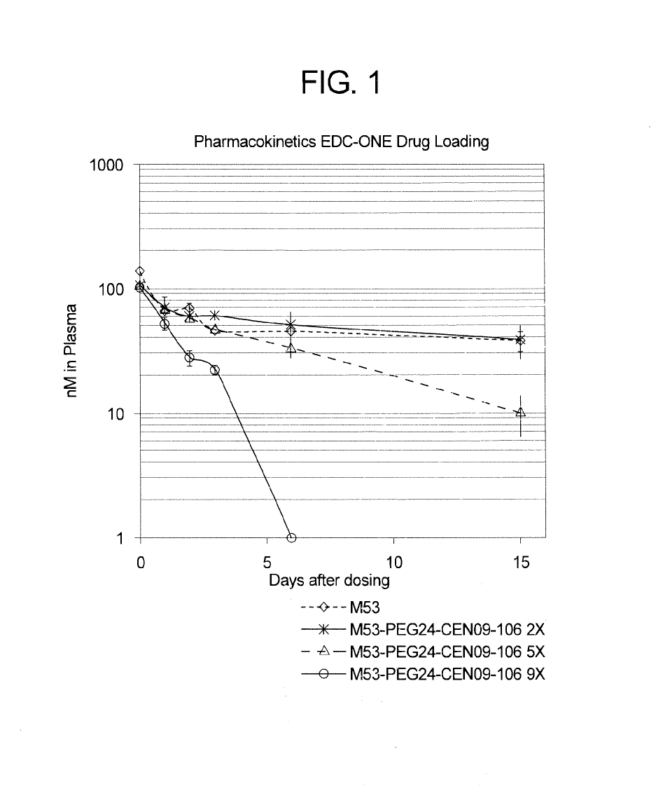

Plasma concentrations of M53-PEG24-CEN09-106 and unconjugated antibody M53 were measured by antigen binding ELISA in the following manner. Antigen capture plates that were prepared by coating wells of 96 well clear bottom ELISA plates with CENP018 (SEQ ID NO: 17) conjugated to BSA at 400 ng BSA-peptide conjugate per well in 100 uL of 200 mM carbonate buffer, pH 9.6 overnight at 4° C. Antigen coated ELISA plates were washed (×3) with PBS pH 7, blocked 30 min with PBS pH 7 containing 1% NFDM (PBS+NFDM), and then washed (×3) with PBS, pH 7. Diluted serum samples and standard curves of unconjugated M53 and each EDC were prepared in the above blocking buffer and applied to coated wells, incubated 30 min at RT, and the wells were washed (×3) with PBS, pH 7. Goat anti-mouse IgG alkaline phosphatase (cat. number: A1418, Sigma-Aldrich, St. Louis, Mo.) was diluted 1:15,000 in PBS+NFDM and added (100 uL) to each well, incubated 30 min at RT, and the wells washed (×3) with PBS, pH 7. PNPP at 1 mg/mL in 1M DEA with 50 mM MgCl₂, pH 9.8 was added (100 uL per well), and the absorbance at 405 nm was determined using a Wallac Victor² Model 1420-041 assay plate reader (Perkin Elmer, Gaithersburg, Md.) every 5 minutes for a total of 6 reads. Absorbance values from known antibody concentration standards were used to determine the concentration of antibody in the serum samples. Those serum concentrations where then plotted to produce the graph shown in FIG. 1.

FIG. 1 shows that exposure (serum half-life) of the EDC (shown as “EDC-ONE” “2x”, “5x”, and “9x”) increased as drug loading decreased. FIG. 1 also shows that a drug loading of 2 provides similar or better serum half-life than free antibody. The serum half-life of detectable (by antigen binding) antibody for unconjugated M53 was determined to be approximately 6 days, while the serum half-life of the detectable antibody for M53-PEG24-CEN09-106 with a drug loading of 2, 5, and 9 was approximately 7, 4, and 2 days, respectively.

In another pharmacokinetic study, unconjugated antibody M53 and EDC M53-PEG24-CEN09-106 with a drug loading of 3 were administered to mice, and the pharmacokinetics of the antibody measured using the methods and ELISA described above. The steroid drug pharmacokinetics of M53-PEG24-CEN09-106 were measured using the drug specific ELISA described below. The comparison between the two ELISA results (antibody concentrations minus drug concentrations) was used to determine the rate of drug breakdown and thus examine M53-PEG24-CEN09-106 stability.

Balb/c mice (three mice/group) were administered with 1 and 10 mg/kg of M53 and M53-PEG24-CEN09-106 with a drug loading of 3 by tail vein injection. Serum was isolated from blood samples acquired by retro-orbital blood collection at 1, 2, 4, 8, 16, 26, and 40 days post-injection. Plasma concentrations of M53-PEG24-CEN09-106 and unconjugated antibody M53 were measured by antigen binding ELISA as described above. Plasma concentrations of the steroid drug in M53-PEG24-CEN09-106 were measured as follows. Antigen coated ELISA plates were washed (×3) with PBS pH 7, blocked 30 min with PBS pH 7 containing 1% NFDM (PBS+NFDM), and then washed (×3) with PBS, pH 7. Diluted serum samples and standard curves of M53-PEG24-CEN09-106 were prepared in the above blocking buffer and applied to coated wells, incubated 30 min at RT, and the wells were washed (×3) with PBS, pH 7. Biotinylated 25C2E3 (a monoclonal IgG1 antibody specific for the steroid portion of PEG24-CEN09-106 and conjugated to biotin [Thermo Scientific, PN 21911]) was diluted to 100 ng/mL in PBS+NFDM and added (100 uL) to each well, incubated 30 min at RT, and the wells washed (×3) with PBS, pH 7. PNPP at 1 mg/mL in 1M DEA with 50 mM MgCl₂, pH 9.8 was added (100 uL per well), and the absorbance at 405 nm was determined using a Wallac Victor² Model 1420-041 assay plate reader (Perkin Elmer, Gaithersburg, Md.) every 5 minutes for a total of 6 reads.

For both ELISAs, absorbance values from known standards were plotted as absorbance over time, generating a linear slope for each concentration. The slopes generated were then plotted against the respective concentrations of the standards, creating a second linear plot. The absorbance values for the unknown serum samples were plotted over time, establishing a slope for unknown sample. The linear estimate from the slope versus concentration plot was then used to extrapolate concentrations for each unknown sample based on their slope.

From these experiments, the serum half-life of the antibody portion of unconjugated M53 was determined to be 10.3 and 11.9 days administered at 10 and 1 mg/kg, respectively. The serum half-life of the antibody portion of M53-PEG24-CEN09-106 was determined to be 9.2 and 11.9 days administered at 10 and 1 mg/kg, respectively. The serum half-life of the steroid drug portion of M53-PEG24-CEN09-106 was determined to be 8 and 11 days administered at 10 and 1 mg/kg, respectively. To determine serum stability of the intact EDC (drug release from the antibody over time) in these samples, the ratio of the slopes of serum decay of the drug portion to the serum decay of the antibody portion were calculated. From these ratios, the serum stability half-life of the EDC when administered at 10 and 1 mg/kg was calculated to be 39 and 45 days, respectively.

The conclusions from these studies are as follows: (1) to obtain maximum exposure of an EDC, a drug loading of 2 or 3 is optimal, although drug loading up to 5 leads to serum half-life only 2 days shorter than the unconjugated antibody; (2) serum breakdown of an EDC is negligible when compared to the EDC's serum half-life; (3) the PEG24-amino-glycoside linkers are non-cleavable linkers; and (4) EDC serum half-life is not greatly affected by dosing levels.

### Example 6

### Tolerated Dose of M53-PEG24-CEN09-106

EDC M53-PEG24-CEN09-106 preparations with drug loading of 2, 5 or 9 drugs per antibody were administered to BALB/c mice (Harlan Laboratories, n=1) in a single dose of 25, 50, 100 and 200 mg/kg via the tail vein to determine single-dose MTDs. Mice were monitored daily for 24 days, and both weight and clinical observations were recorded (weight measured at least twice a week and evaluation for overt signs of toxicity conducted at least twice a day). The MTD was defined as the highest dose that did not cause serious overt toxicities or >20% weight loss in any of the animals.

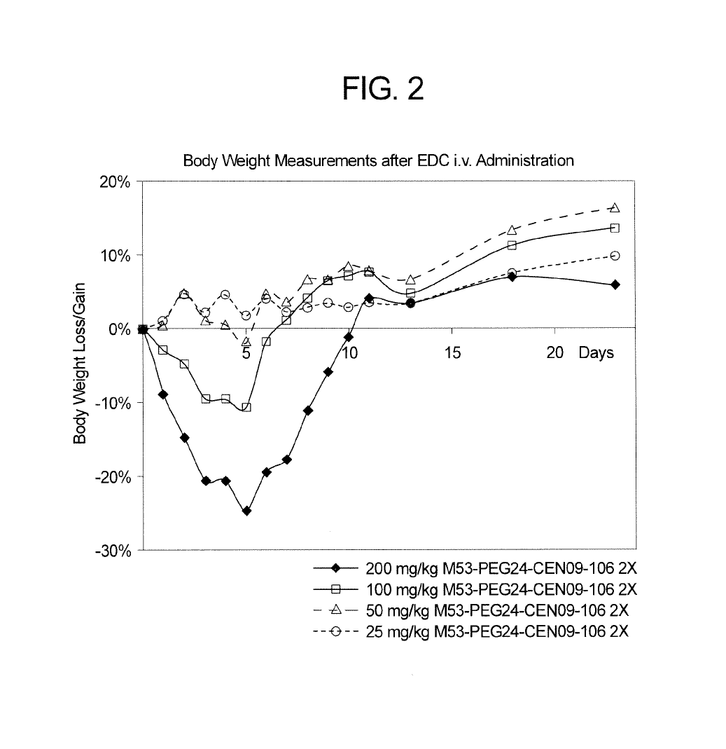

For the M53-PEG24-CEN09-106 with a drug loading of 2 drugs per antibody, percent weight gain or loss was measured, plotted against day of dose and graphed, and shown in FIG. 2. The MTD was determined to be >100 mg/kg, which was the highest dose that did not induce >20% weight loss, severe signs of distress, or overt toxicities in any of the animals. At the 200 mg/kg dose, mice experienced limb weakness at day 1 but recovered by day 3 and experienced a 25% loss in body weight.

The single-dose tolerability of M53-PEG24-CEN09-106 with a drug loading of 5 drugs per antibody was determined to be >50 mg/kg using the same criteria. At 25 and 50 mg/kg, no signs of toxicity or weight loss were observed. At the 100 mg/kg dose, mice lost 27% of their weight and experienced slight limpness and closed front limbs but recovered by day 3. At the 200 mg/kg dose, mice experienced limpness and closed front limbs and weakness at days 1 and 2.

The single-dose tolerability of M53-PEG24-CEN09-106 with a drug loading of 9 drugs per antibody was determined to be >25 mg/kg using the same criteria. At 25 mg/kg no signs of toxicity or weight loss were observed. At the 50 mg/kg dose, mice lost 30% of their weight by day 5, which returned to normal by day 10, and experienced slight limpness on day 2 but recovered by day 3. At the 100 and 200 mg/kg doses, mice experienced limpness, closed front limbs and weakness by day 1 which continued through day 2.

In a separate experiment, male BALB/c mice (BALB/cAnNHsd, Harlan Laboratories) were administered a single and immediate dose of 500 mg/kg of M53-PEG24-CEN09-106 with a drug loading of 2 drugs per antibody by intraperitoneal injection. Animal weights were recorded over a 22 day period and visual observations were made over a period of 146 days and compared to mice receiving a vehicle control. Weight measurements showed an average loss in body weight of 30% over the first 6 days post injection but visual observations showed no overt signs of toxicity. Mice regained normal control body weight by day 15.

These results indicate that the route of administration can affect most tolerated dose as 500 mg/kg M53-PEG24-CEN09-106 with a drug loading of 2 drugs per antibody administered intraperitoneally is tolerated better (no limb weakness) as 200 mg/kg M53-PEG24-CEN09-106 with 2 agents per antibody administered a single i.v. bolus. This could also be due to the slower release into the blood stream with i.p. administration.

The conclusions from the studies in Examples 5 and 6 are as follows: (1) drug loading affects the tolerability of the EDC, and 2 drugs per antibody shows a tolerability in mice of up to 500 mg/kg and 9 drugs per antibody shows a tolerability between 25 and 50 mg/kg; (2) antibody loading of 2 to 4 agents is optimal to maintain minimal toxicity and maximal pharmacokinetics; (3) the route of administration and/or the speed at which EDCs enter the blood stream can affect MTD, with slower blood stream administration being more tolerated; and (4) weight loss and overt toxicity are effected by high levels of dosing and high levels of drug loading. These results also demonstrate that a tolerated human dose could be higher than 10 mg/kg.

### Example 7

### Efficacy of M53-PEG24-CEN09-106 in A549 and H460 Xenograft Models

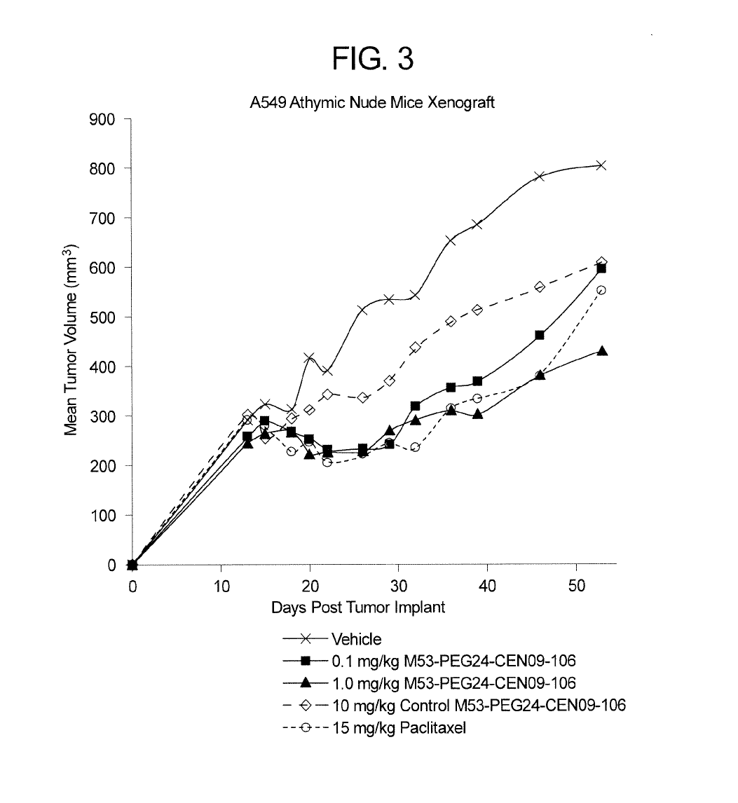

The efficacy of M53-PEG24-CEN09-106 with a drug loading of 8 steroid drugs per antibody was demonstrated in an A549 xenograft model. Briefly, to establish a non-small-cell lung cancer disease model, 6×10⁶ A549 cells in 200 μL, RPMI1640+50% Matrigel HC (BD Biosciences, San Jose, Calif.) were implanted into the left flank of Hsd:Athymic Nude-Foxn1^{nu} mice (Harlan, Indianapolis, Ind.). Therapy with antibody-drug conjugates was then initiated when the tumor volume in groups of 5 animals averaged ˜300 mm³. Treatment using vehicle control, 0.1 and 1 mg/kg M53-PEG24-CEN09-106, and 10 mg/kg control M53-PEG24-CEN09-106 (a control conjugate with a drug loading of 7 drugs per antibody where the antibody's target is not on the cell surface but drug and linker and antibody isotype are the same as M53-PEG24-CEN09-106) were all administered i.v. using the schedule of one injection every 7 days with 3 total injected doses (q7d×3). 15 mg/kg paclitaxel dosed at q2d×5 served as a positive control treatment group. Using this schedule, tumor volumes were measured for each group using calibrated vernier calipers and plotted against first day of tumor implant for 54 days post-implant and 40 days post-initial dose, to produce the graph shown in FIG. 3. The results show that M53-PEG24-CEN09-106 at 1 mg/kg produced 65% growth inhibition of the tumor when compared to vehicle. Paclitaxel at its optimum dosing produced 45% growth inhibition of the tumor when compared to vehicle. At 0.1 mg/kg with the same schedule, M53-PEG24-CEN09-106 produced 35% growth inhibition of the tumor when compared to vehicle. At 10 mg/kg with the same schedule, control M53-PEG24-CEN09-106 produced 35% growth inhibition of the tumor when compared to vehicle.

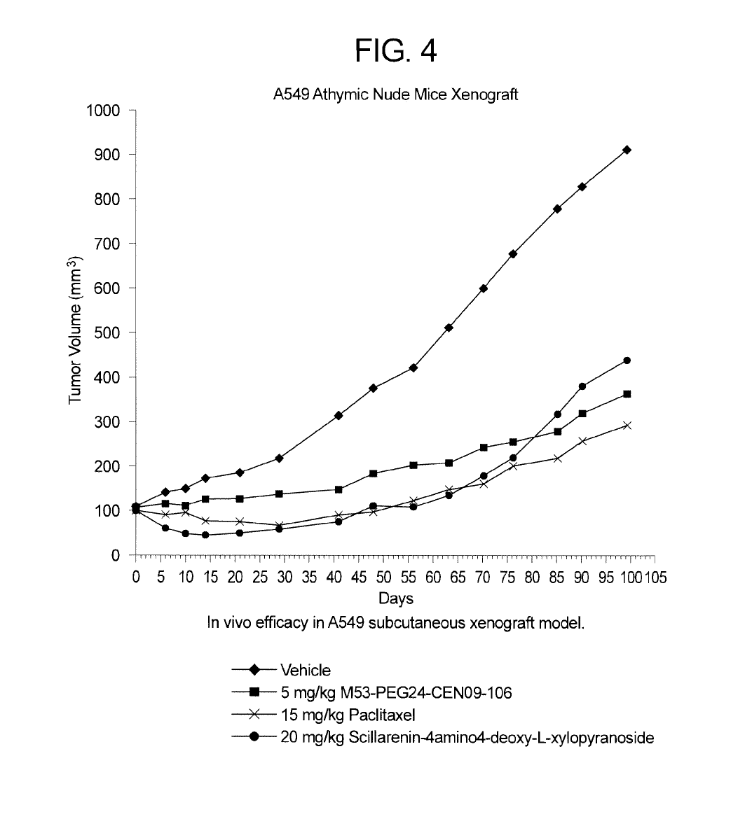

In a second A549 study, the efficacy of M53-PEG24-CEN09-106 with a drug loading of 3 agents per antibody was demonstrated. Briefly, to establish a non-small-cell lung cancer disease model, subcutaneous A549 xenografts were initiated in female HRLN nu/nu mice by implanting 8 mm³ A549 tumor fragments subcutaneously into the left flank. Tumor growth was monitored, and mice bearing tumors of 60-180 mm³ were selected for the study. Tumor-bearing mice (n=7 mice/group) with a group average tumor volume of approximately 110 mm³ were treated i.p. with 5 mgs/kg M53-PEG24-CEN09-106 at q3d×4, 20 mg/kg scillarenin-4-amino-deoxy-L-xylopryanoside (an intermediate in the construction of M53-PEG24-CEN09-106 and known cytotoxin) at q2dx5, and 15 mg/kg paclitaxel at q1d×5. Tumor volumes were measured for each group using calibrated vernier calipers plotted against day of first dose for 100 days to produce the graph shown in FIG. 4.

Using this schedule, after 100 days post-initial dose, M53-PEG24-CEN09-106 at 5 mg/kg produced 72% growth inhibition of the tumor when compared to vehicle. Paclitaxel at its optimum dosing produced 80% growth inhibition of the tumor when compared to vehicle. At 20 mg/kg scillarenin-4-amino-deoxy-L-xylopryanoside produced 60% growth inhibition of the tumor when compared to vehicle (see FIG. 4). Mice administered paclitaxel showed 11% weight loss and scillarenin-4-amino-deoxy-L-xylopyranoside showed 8% weight loss while all other mice showed a slight weight gain. This study shows that 5 mg/kg of M53-PEG24-CEN09-106 with a drug loading of 3 agents per antibody shows efficacy at slowing tumor growth in a similar fashion to paclitaxel at its optimal dosing. The study also demonstrates that the efficacy of M53-PEG24-CEN09-106 is similar to scillarenin-4-amino-deoxy-L-xylopyranoside even when administered at a total molar level 427-fold lower. In addition, M53-PEG24-CEN09-106 produced no weight loss, demonstrating it is less toxic than the paclitaxel or scillarenin-4-amino-deoxy-L-xylopyranoside. These results also demonstrate that efficacious human dosing can be in a range encompassing 5 mg/kg.

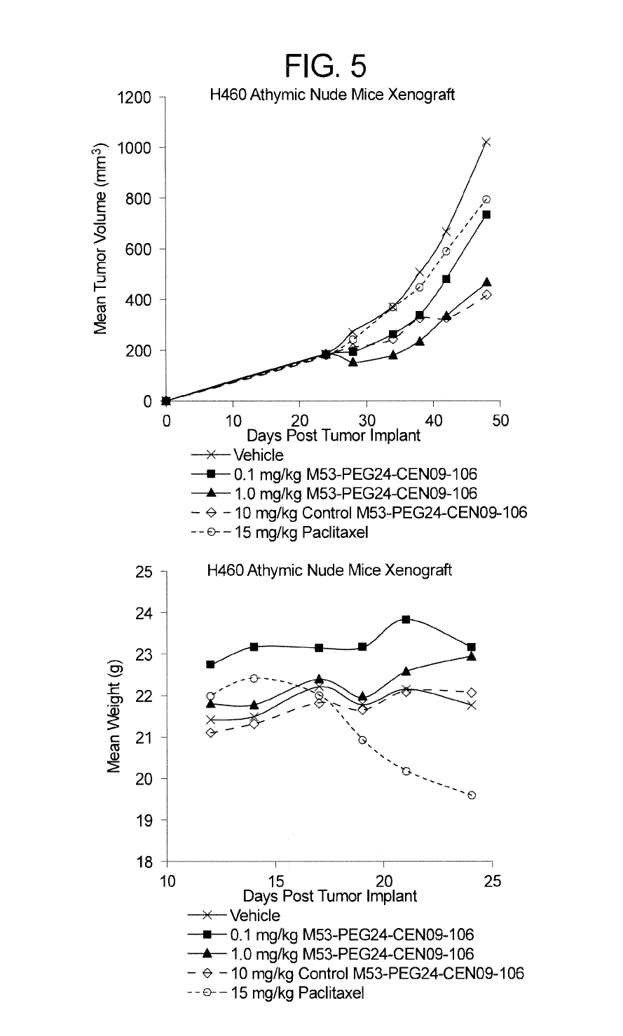

In another study, the efficacy of M53-PEG24-CEN09-106 with a drug loading of 8 agents per antibody was demonstrated in an H460 xenograft model. Briefly, to establish a large-cell lung cancer disease model, 1×10⁶ H-460 cells in 100 μL RPMI1640+30% Matrigel HC (BD Biosciences, San Jose, Calif.) were implanted into the left flank of Hsd:Athymic Nude-Foxn1^{nu} mice (Harlan, Indianapolis, Ind.). Therapy was initiated when the tumor size in groups of 5 animals averaged ˜200 mm³. Treatment using vehicle control, M53-PEG24-CEN09-106 at 0.1 and 1 mg/kg, and 10 mg/kg control M53-PEG24-CEN09-106 (as described above) consisted of multiple i.v. injections using the schedule of one injection every 7 days for 2 injections (q7d×2). Paclitaxel served as a positive control treatment group and was dosed i.v. at 15 mg/kg using the schedule of one injection every 2 days for 5 injections (q2d×5). Using this schedule, tumor volumes were measured for each group using calibrated vernier calipers and plotted against day of tumor implant for 24 days post-implant and 12 days post-initial dose, graphed and shown in FIG. 5. The results show that M53-PEG24-CEN09-106 at 1 mg/kg produced 73% growth inhibition of the tumor when compared to vehicle. At 0.1 mg/kg with the same schedule, M53-PEG24-CEN09-106 produced 38% growth inhibition of the tumor when compared to vehicle and no weight loss. Paclitaxel at its optimum dosing produced 78% growth inhibition of the tumor when compared to vehicle and an average weight loss of 15%. At 10 mg/kg with the same schedule, control M53-PEG24-CEN09-106 produced 33% growth inhibition of the tumor when compared to vehicle.

### Example 8

### Efficacy of M53-PEG24-CEN09-106 Combination Therapy in PANC-1 Xenograft Model

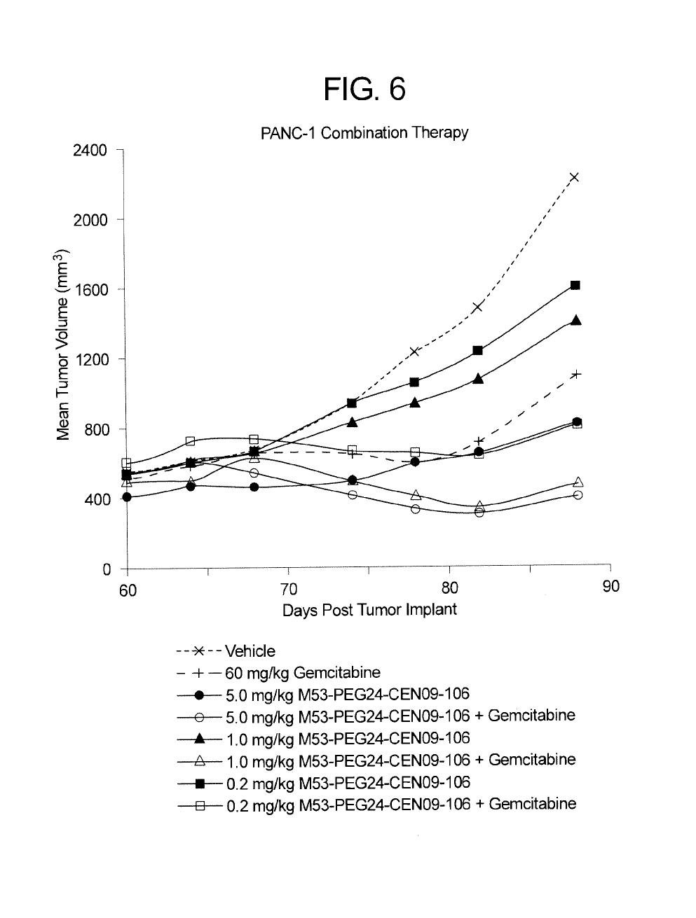

In another study, the efficacy of M53-PEG24-CEN09-106 with a drug loading of 8 agents per antibody was demonstrated in combination with gemcitabine in a PANC-1 xenograft model. A disease model of pancreatic cancer was established by implanting 5×10⁶ PANC-1 cells in 100 μL DMEM into the left flank of 7 week old female C.B-17/IcrHsd-Prkdc^{scid}Lyst^{bg} mice. Mice were then treated with M53-PEG24-CEN09-106 with a drug loading of 8 and/or gemcitabine when the tumor size in groups of 3 animals averaged ˜500 mm³. Treatments consisted of either vehicle control, M53-PEG24-CEN09-106 at 0.2, 1 and 5 mg/kg, with and without 60 mg/kg gemcitabine or 60 mg/kg gemcitabine alone and consisted of multiple injections using the M53-PEG24-CEN09-106 schedule of one injection every 7 days for 3 injections (q7d×3) administered i.v. and the gemcitabine schedule of one injection every 3 days for 5 injections (q3d×5) administered i.p. Tumor volumes were measured for each group using calibrated vernier calipers and plotted against first day of tumor implant for 28 days post-initial dose, graphed and shown in FIG. 6. At day 28 post-initial dose, M53-PEG24-CEN09-106 at 0.2, 1, 5 mg/kg produced 38%, 48% and 76% growth inhibition of the tumor, respectively, when compared to vehicle. When gemcitabine (one injection every 3 days for 5 injections) was combined with the same M53-PEG24-CEN09-106 schedules and dosing (0.2, 1, 5 mg/kg), the dosing produced 88% growth inhibition for 0.2 mg/kg and tumor regression for the other combined doses. Gemcitabine alone produced 68% growth inhibition of the tumor when compared to vehicle.

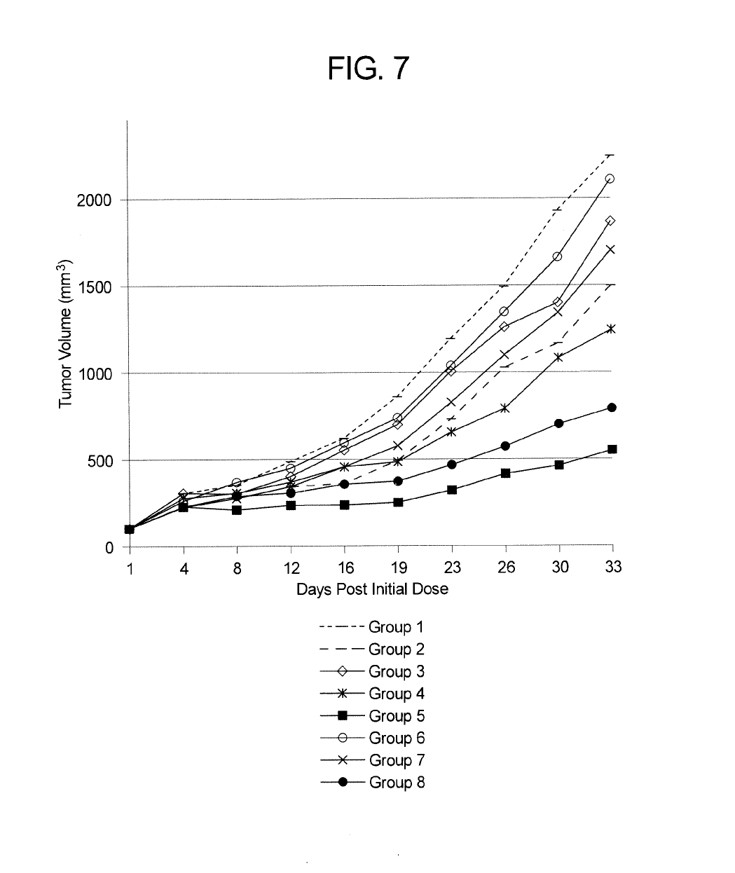

In another study, the Panc-1 subcutaneous xenograft model was used to evaluate antitumor activity of M53-PEG24-CEN09-106 with a drug loading of 7 drugs per antibody. Subcutaneous Panc-1 xenografts were initiated in female HRLN nu/nu mice by implanting 1 mm³ PANC-1 tumor fragments were implanted subcutaneously into the right flank, tumor growth was monitored and mice bearing tumors of 80-120 mm³ were selected for the study. Tumor-bearing mice (n=10 mice/group) were treated as shown in the table below. Tumor volume was measured for each group 2× per week using calibrated vernier calipers plotted against day of first dose to produce the graph shown in FIG. 7. Among the treatment groups, no toxic effects were observed (toxicity determined as >10% weight loss during study period). The conclusion from this study was that M53-PEG24-CEN09-106 administration to mice harboring a solid tumor developed from the human pancreatic cell line PANC1 leads to tumor reduction in a dose dependent manner when compared to the vehicle group 1.

| | | Drug or Test Agent; Dose; Route; and Schedule | | | |
|---|---|---|---|---|---|
| Group | N | Agent | mg/kg | Route | Schedule |
| 1 | 10 | Vehicle | — | iv | Q3Dx7 |
| 2 | 10 | Gemcitabine | 120 | ip | Q3Dx7 |
| 3 | 10 | M53-PEG24-CEN09-106 | 1 | iv | Q3Dx7 |
| 4 | 10 | M53-PEG24-CEN09-106 | 5 | iv | Q3Dx7 |
| 5 | 10 | M53-PEG24-CEN09-106 | 15 | iv | Q3Dx7 |
| 6 | 10 | M53-PEG24-CEN09-106 | 1 | iv | Q7Dx3 |
| 7 | 10 | M53-PEG24-CEN09-106 | 5 | iv | Q7Dx3 |
| 8 | 10 | M53-PEG24-CEN09-106 | 15 | iv | Q7Dx3 |

1. An extracellular targeted drug conjugate (EDC) comprising an antibody that binds to the dysadherin subunit of the Na,K-ATPase signaling complex covalently bound to a polyethylene glycol (PEG) amino-glycoside linker covalently attached to a drug that is either digitoxigenin or scillarenin, wherein said PEG portion of said linker contains from 2 to 36 glycol units, said amino-glycoside is 4-amino-riboside or 4-amino-xyloside, and said drug is attached to said linker via a C1 hydroxyl group of the amino-glycoside, and wherein said conjugate contains from 2 to 8 drugs.

2. The EDC of claim 1 that contains 3 drugs.

3. The EDC of claim 1 that contains 7 drugs.

4. The EDC of claim 1, wherein the PEG portion of the linker contains 24 glycol units.

5. The EDC of claim 1, wherein the antibody is an M53 monoclonal antibody.

6. The EDC of claim 1 wherein the antibody is a chimeric or humanized antibody that comprises a heavy or light chain variable region of M53 and a human constant region.

7. The EDC of claim 1, wherein the antibody comprises one or more heavy chain CDRs selected from the group consisting of SEQ ID NOS: 33-35, and/or one or more light chain CDRs selected from the group consisting of SEQ ID NOS: 36-38.

8. A pharmaceutical formulation of an EDC of any of claims 1 to 7 suitable for intravenous administration that comprises a pharmaceutically acceptable vehicle, vector, diluent, and/or excipient.

9. A unit dose form of the pharmaceutical formulation of claim 7 that contains from about 5 mg to about 5 g of said EDC.

10. A method of treating a patient with cancer that comprises administering a therapeutically effective dose of an EDC of claim 1 to a patient in need of treatment.

11. The method of claim 9, further comprising administering a second drug to said patient, wherein said second drug is selected from the group consisting of gemcitabine, a TRAIL (tissue necrosis factor (TNF)-related apoptosis-inducing ligand), and a fibroblast growth factor receptor kinase inhibitor.

12. The method of claim 10 or 11, wherein said patient is a lung cancer patient.

13. The method of claim 10 or 11, wherein said patient is a pancreatic cancer patient.

14. The method of claim 10 or 11, wherein said patient is a lymphoma cancer patient.

15. The method of claim 12, wherein said patient is administered a second drug that is either a TRAIL or a fibroblast growth factor receptor kinase inhibitor.

16. The method of claim 13, wherein said patient is administered gemcitabine in combination with said EDC.

17. The method of claim 10, wherein said patient is administered said EDC at a dose in the range of 0.1 mg per kg patient weight (“mg/kg”) to 10 mg/kg.

18. The method of claim 17, wherein said dose is administered once per week or once every three weeks.
